(19) United States
(12) Patent Application Publication
(10) Pub. No.: US 2026/0035616 A1
(43) Pub. Date: Feb. 5, 2026

(54) LIQUID CRYSTAL COMPOSITION, MONOFUNCTIONAL MONOMER, LIQUID CRYSTAL CURED LAYER, OPTICAL FILM, POLARIZING PLATE, AND IMAGE DISPLAY DEVICE

(71) Applicant: FUJIFILM Corporation, Tokyo (JP)

(72) Inventors: Hiroshi INADA, Minamiashigara-shi (JP); Ryoji GOTO, Minamiashigara-shi (JP)

(73) Assignee: FUJIFILM Corporation, Tokyo (JP)

(21) Appl. No.: 19/357,132

(22) Filed: Oct. 14, 2025

Related U.S. Application Data
(63) Continuation of application No. PCT/JP2024/016513, filed on Apr. 26, 2024.

(30) Foreign Application Priority Data
May 22, 2023 (JP) 2023-083741

(51) Int. Cl. C09K 19/38; C07D 417/04; C08F 222/24; C09K 19/04; G02B 5/30

(52) U.S. Cl. CPC C09K 19/3861; C07D 417/04; C08F 222/24; G02B 5/3016; C08F 2800/20; C09K 2019/0448

(57) ABSTRACT

An object of the present invention is to provide a liquid crystal composition, a monofunctional monomer, a liquid crystal cured layer, an optical film, a polarizing plate, and an image display device, in which precipitation is suppressed and aligning properties are also excellent. The liquid crystal composition of the present invention is a liquid crystal composition containing a compound represented by Formula (A) and a liquid crystal compound.

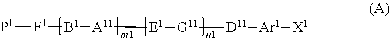

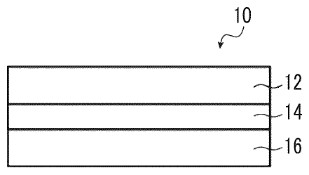

### CROSS-REFERENCE TO RELATED APPLICATIONS

This application is a Continuation of PCT International Application No. PCT/JP2024/016513, filed on Apr. 26, 2024, which claims priority under 35 U.S.C. § 119 (a) to Japanese Patent Application No. 2023-083741, filed on May 22, 2023. The above applications are hereby expressly incorporated by reference, in their entirety, into the present application.

### BACKGROUND OF THE INVENTION

### 1. Field of the Invention

The present invention relates to a liquid crystal composition, a monofunctional monomer, a liquid crystal cured layer, an optical film, a polarizing plate, and an image display device.

### 2. Description of the Related Art

Optical films such as an optical compensation sheet and a phase difference film have been used in various image display devices in order to eliminate image coloration or expand a viewing angle.

A stretched birefringent film has been used as the optical film, but in recent years, it has been proposed to use an optical film having an optically anisotropic layer consisting of a liquid crystal compound instead of the stretched birefringent film.

As such an optical film, an optical film containing a cured substance of a liquid crystal composition which contains a compound represented by a predetermined formula and a polymerizable liquid crystal compound is known (for example, see JP2018-077465A).

### SUMMARY OF THE INVENTION

As a result of studying the liquid crystal composition disclosed in JP2018-077465A, the present inventors have found that the liquid crystal composition has an effect of suppressing precipitation, but there is room for improvement in aligning properties.

Therefore, an object of the present invention is to provide a liquid crystal composition, a monofunctional monomer, a liquid crystal cured layer, an optical film, a polarizing plate, and an image display device, in which precipitation is suppressed and aligning properties are also excellent.

As a result of intensive studies to achieve the above-described object, the present inventors have found that, in a case where a liquid crystal composition containing a compound represented by Formula (A) and a liquid crystal compound is used, the precipitation is suppressed and the aligning properties are also excellent, and have completed the present invention.

That is, the present inventors have found that the above-described object can be achieved by employing the following configurations.

[1] A liquid crystal composition comprising:
a compound represented by Formula (A) described later; and
a liquid crystal compound.
   [2] The liquid crystal composition according to [1],
in which the liquid crystal compound is a compound represented by Formula (B) described later.
   [3] The liquid crystal composition according to [1] or [2],
in which a content of the compound represented by Formula (A) described later is 18% by mass or less with respect to a total mass of the compound represented by Formula (A) described later and the liquid crystal compound.
   [4] The liquid crystal composition according to any one of [1] to [3],
in which Ar¹ in Formula (A) described later is the aromatic ring represented by Formula (Ar-1) or (Ar-2) described later.
   [5] The liquid crystal composition according to any one of [2] to [4],
in which Ar¹ in Formula (A) described later has the same structure as Ar² in Formula (B) described later.
   [6] The liquid crystal composition according to any one of [2] to [5],
in which G¹¹ in Formula (A) described later has the same structure as G¹ in Formula (B) described later, and

A¹¹ in Formula (A) described later has the same structure as A¹ in Formula (B) described later.

[7] A monofunctional monomer represented by Formula (A) described later.
[8] A liquid crystal cured layer obtained by fixing an alignment state of the liquid crystal composition according to any one of [1] to [6].
[9] An optical film comprising:
the liquid crystal cured layer according to [8].
   [10] A polarizing plate comprising:
the optical film according to [9]; and
a polarizer.
   [11] An image display device comprising:
the optical film according to [9].

According to the present invention, it is possible to provide a liquid crystal composition, a monofunctional monomer, a liquid crystal cured layer, an optical film, a polarizing plate, and an image display device, in which precipitation is suppressed and aligning properties are also excellent.

### BRIEF DESCRIPTION OF THE DRAWINGS

The FIGURE is a schematic cross-sectional view showing an example of an optical film.

### DESCRIPTION OF THE PREFERRED EMBODIMENTS

Hereinafter, the present invention will be described in detail.

The description of configuration requirements described below may be made based on typical embodiments of the present invention, but the present invention is not limited to such embodiments.

Any numerical range expressed using “to” in the present specification refers to a range including the numerical values before and after the “to” as a lower limit value and an upper limit value, respectively.

In addition, in a range of numerical values described in stages in the present specification, the upper limit value or the lower limit value described in a certain range of numerical values may be replaced with an upper limit value or a lower limit value of the range of numerical values described in other stages. In addition, regarding the numerical range described in the present specification, an upper limit value or a lower limit value described in a numerical value may be replaced with a value described in Examples.

In addition, in the present specification, substances corresponding to respective components may be used alone or in combination of two or more kinds thereof. Here, in a case where two or more kinds of substances are used in combination for each component, the content of the component indicates the total content of the substances used in combination, unless otherwise specified.

In addition, in the present specification, “(meth)acrylate” denotes “acrylate” or “methacrylate”, “(meth)acryl” denotes “acryl” or “methacryl”, and “(meth)acryloyl” denotes “acryloyl” or “methacryloyl”.

In addition, in the present specification, a bonding direction of a divalent group (for example, —O—CO—) described is not particularly limited, and for example, in a case where L² in an “L¹-L²-L³” bond is —O—CO—, and a bonding position on the L¹ side is represented by *1 and a bonding position on the L³ side is represented by *2, L² may be *1-O—CO-*2 or *1-CO—O—*2.

In addition, in the present specification, Re(λ) and Rth(λ) respectively represent an in-plane retardation at a wavelength 2 and a thickness-direction retardation at a wavelength 2. Unless otherwise specified, the wavelength λ refers to 550 nm.

In the present invention, Re(λ) and Rth(λ) are values measured at the wavelength of A in AxoScan (manufactured by Axometrics, Inc.). By inputting an average refractive index ((nx+ny+nz)/3) and a film thickness (d (μm)) in AxoScan,
a slow axis direction (°),

$$Re ( λ ) = R 0 ( λ ) , and$$

$$Rth ( λ ) = ( ( n x + ny ) / 2 - nz ) \times d$$

are calculated.

Although R0 (λ) is displayed as a numerical value calculated by AxoScan, it means Re (λ).

In addition, in the present specification, examples of a substituent (monovalent substituent) include substituents described in the following substituent group A.

In the present specification, “may have a substituent” includes not only an aspect of not having a substituent but also an aspect of having one or more substituents.

### Substituent Group A

Examples of the substituent include:
a halogen atom (for example, a fluorine atom, a chlorine atom, or a bromine atom, preferably a chlorine atom or a fluorine atom, and more preferably a fluorine atom);
an alkyl group (a linear, branched, or cyclic alkyl group having preferably 1 to 48 carbon atoms, more preferably 1 to 24 carbon atoms, and particularly preferably 1 to 8 carbon atoms, such as a linear alkyl group having 1 to 6 carbon atoms (for example, a methyl group, an ethyl group, a n-propyl group, a n-butyl group, a n-pentyl group, and a n-hexyl group), a branched alkyl group having 3 to 6 carbon atoms (for example, an isopropyl group, an isobutyl group, a tert-butyl group, a sec-butyl group, a neopentyl group, an isohexyl group, and a 3-methylpentyl group), and a cyclic alkyl group having 3 to 12 carbon atoms (for example, a cyclopropyl group, a cyclopentyl group, a cyclohexyl group, a 1-norbornyl group, and a 1-adamantyl group));
an alkenyl group (an alkenyl group having preferably 2 to 48 carbon atoms and more preferably 2 to 18 carbon atoms, such as a vinyl group, an allyl group, a 1-butenyl group, and a 2-butenyl group);
an alkynyl group (an alkynyl group having preferably 2 to 6 carbon atoms and more preferably 2 to 4 carbon atoms, such as an ethynyl group, a 1-propynyl group, a propargyl group, a 1-butynyl group, and a 2-butynyl group);
an aryl group (an aryl group having preferably 6 to 48 carbon atoms and more preferably 6 to 24 carbon atoms, such as a phenyl group, an oligoaryl group (a naphthyl group or an anthryl group), a phenanthrenyl group, a fluorenyl group, a pyrenyl group, a triphenylenyl group, and a biphenyl group);
a heteroaryl group (a heterocyclic group having preferably 1 to 32 carbon atoms and more preferably 1 to 18 carbon atoms, such as a 2-thienyl group, a 4-pyridyl group, a 2-furyl group, a 2-pyrimidinyl group, a 1-pyridyl group, a 2-benzothiazolyl group, a 1-imidazolyl group, a 1-pyrazolyl group, and a benzotriazol-1-yl group);
an arylalkyl group (an arylalkyl group having preferably 7 to 15 carbon atoms, such as a benzyl group, a phenethyl group, a methylbenzyl group, a phenylpropyl group, a 1-methylphenylethyl group, a phenylbutyl group, a 2-methylphenylpropyl group, a tetrahydronaphthyl group, a naphthylmethyl group, a naphthylethyl group, an indenyl group, a fluorenyl group, an anthracenylmethyl group (an anthrylmethyl group), and a phenanthrenylmethyl group (a phenanthrylmethyl group));
a silyl group (a silyl group having preferably 3 to 38 carbon atoms and more preferably 3 to 18 carbon atoms, such as a trimethylsilyl group, a triethylsilyl group, a tributylsilyl group, a t-butyldimethylsilyl group, and a t-hexyldimethylsilyl group);
a hydroxy group; a cyano group; a nitro group; a morpholino group;
an alkoxy group (an alkoxy group having preferably 1 to 48 carbon atoms and more preferably 1 to 24 carbon atoms, such as a methoxy group, an ethoxy group, a 1-butoxy group, a 2-butoxy group, an isopropoxy group, a t-butoxy group, a dodecyloxy group, and a cycloalkyloxy group (for example, a cyclopentyloxy group or a cyclohexyloxy group));
an aryloxy group (an aryloxy group having preferably 6 to 48 carbon atoms and more preferably 6 to 24 carbon atoms, such as a phenoxy group and a 1-naphthoxy group);
an alkenyloxy group (an alkenyloxy group having preferably 2 to 6 carbon atoms, such as a vinyloxy group, a 1-propenyloxy group, a 2-n-propenyloxy group (an allyloxy group), a 1-n-butenyloxy group, and a prenyloxy group);
a heterocyclic oxy group (a heterocyclic oxy group having preferably 1 to 32 carbon atoms and more preferably 1 to 18 carbon atoms, such as a 1-phenyltetrazole-5-oxy group and a 2-tetrahydropyranyloxy group);
a silyloxy group (a silyloxy group having preferably 1 to 32 carbon atoms and more preferably 1 to 18 carbon atoms, such as a trimethylsilyloxy group, a t-butyldimethylsilyloxy group, and a diphenylmethylsilyloxy group);
an acyloxy group (an acyloxy group having preferably 2 to 48 carbon atoms and more preferably 2 to 24 carbon atoms, such as an acetoxy group, a pivaloyloxy group, a benzoyloxy group, a dodecanoyloxy group, an acryloyloxy group, and a methacryloyloxy group);
a hydroxyalkyleneoxy group (a hydroxyalkyleneoxy group having preferably 2 to 10 carbon atoms, such as a hydroxyethyleneoxy group);
an alkoxycarbonyloxy group (an alkoxycarbonyloxy group having preferably 2 to 48 carbon atoms and more preferably 2 to 24 carbon atoms, such as an ethoxycarbonyloxy group, a t-butoxycarbonyloxy group, and a cycloalkyloxycarbonyloxy group (for example, a cyclohexyloxycarbonyloxy group));
an aryloxycarbonyloxy group (an aryloxycarbonyloxy group having preferably 7 to 32 carbon atoms and more preferably 7 to 24 carbon atoms, such as a phenoxycarbonyloxy group);
a carbamoyloxy group (a carbamoyloxy group having preferably 1 to 48 carbon atoms and more preferably 1 to 24 carbon atoms, such as an N,N-dimethylcarbamoyloxy group, an N-butylcarbamoyloxy group, an N-phenylcarbamoyloxy group, and an N-ethyl-N-phenylcarbamoyloxy group);
a sulfamoyloxy group (a sulfamoyloxy group having preferably 1 to 32 carbon atoms and more preferably 1 to 24 carbon atoms, such as an N,N-diethylsulfamoyloxy group and an N-propylsulfamoyloxy group);
an alkylsulfonyloxy group (an alkylsulfonyloxy group having preferably 1 to 38 carbon atoms and more preferably 1 to 24 carbon atoms, such as a methylsulfonyloxy group, a hexadecylsulfonyloxy group, and a cyclohexylsulfonyloxy group);
an arylsulfonyloxy group (an arylsulfonyloxy group having preferably 6 to 32 carbon atoms and more preferably 6 to 24 carbon atoms, such as a phenylsulfonyloxy group);
an acyl group (an acyl group having preferably 1 to 48 carbon atoms and more preferably 1 to 24 carbon atoms, such as a formyl group, an acetyl group, an acryloyl group, a methacryloyl group, a pivaloyl group, a benzoyl group, a tetradecanoyl group, and a cyclohexanoyl group);
an alkoxycarbonyl group (an alkoxycarbonyl group having preferably 2 to 48 carbon atoms and more preferably 2 to 24 carbon atoms, such as a methoxycarbonyl group, an ethoxycarbonyl group, an octadecyloxycarbonyl group, a cyclohexyloxycarbonyl group, and a 2,6-di-tert-butyl-4-methylcyclohexyloxycarbonyl group);
an aryloxycarbonyl group (an aryloxycarbonyl group having preferably 7 to 32 carbon atoms and more preferably 7 to 24 carbon atoms, such as a phenoxycarbonyl group);
a carbamoyl group (a carbamoyl group having preferably 1 to 48 carbon atoms and more preferably 1 to 24 carbon atoms, such as a carbamoyl group, an N,N-diethylcarbamoyl group, an N-ethyl-N-octylcarbamoyl group, an N,N-dibutylcarbamoyl group, an N-propylcarbamoyl group, an N-phenylcarbamoyl group, an N-methyl-N-phenylcarbamoyl group, and an N,N-dicyclohexylcarbamoyl group);
an amino group (an amino group having preferably 32 or less carbon atoms and more preferably 24 or less carbon atoms, such as an amino group, a methylamino group, an N,N-dibutylamino group, a tetradecylamino group, a 2-ethylhexylamino group, and a cyclohexylamino group);
an anilino group (an anilino group having preferably 6 to 32 carbon atoms and more preferably 6 to 24 carbon atoms, such as an anilino group and an N-methylanilino group);
a heterocyclic amino group (a heterocyclic amino group having preferably 1 to 32 carbon atoms and more preferably 1 to 18 carbon atoms, such as a 4-pyridylamino group);
a carbonamide group (a carbonamide group having preferably 2 to 48 carbon atoms and more preferably 2 to 24 carbon atoms, such as an acetamide group, a benzamide group, a tetradecaneamide group, a pivaloylamide group, and a cyclohexaneamide group);
a ureido group (a ureido group having preferably 1 to 32 carbon atoms and more preferably 1 to 24 carbon atoms, such as a ureido group, an N,N-dimethylureido group, and an N-phenylureido group);
an imide group (an imide group having preferably 36 or less carbon atoms and more preferably 24 or less carbon atoms, such as an N-succinimide group and an N-phthalimide group);
an alkoxycarbonylamino group (an alkoxycarbonylamino group having preferably 2 to 48 carbon atoms and more preferably 2 to 24 carbon atoms, such as a methoxycarbonylamino group, an ethoxycarbonylamino group, a t-butoxycarbonylamino group, an octadecyloxycarbonylamino group, and a cyclohexyloxycarbonylamino group);
an aryloxycarbonylamino group (an aryloxycarbonylamino group having preferably 7 to 32 carbon atoms and more preferably 7 to 24 carbon atoms, such as a phenoxycarbonylamino group);
a sulfonamide group (a sulfonamide group having preferably 1 to 48 carbon atoms and more preferably 1 to 24 carbon atoms, such as a methanesulfonamide group, a butanesulfonamide group, a benzenesulfonamide group, a hexadecanesulfonamide group, and a cyclohexanesulfonamide group);
a sulfamoylamino group (a sulfamoylamino group having preferably 1 to 48 carbon atoms and more preferably 1 to 24 carbon atoms, such as an N,N-dipropylsulfamoylamino group and an N-ethyl-N-dodecylsulfamoylamino group);
an azo group (an azo group having preferably 1 to 32 carbon atoms and more preferably 1 to 24 carbon atoms, such as a phenylazo group and a 3-pyrazolylazo group);
an alkylthio group (an alkylthio group having preferably 1 to 48 carbon atoms and more preferably 1 to 24 carbon atoms, such as a methylthio group, an ethylthio group, an octylthio group, and a cyclohexylthio group);
an arylthio group (an arylthio group having preferably 6 to 48 carbon atoms and more preferably 6 to 24 carbon atoms, such as a phenylthio group);
a heterocyclic thio group (a heterocyclic thio group having preferably 1 to 32 carbon atoms and more preferably 1 to 18 carbon atoms, such as a 2-benzothiazolylthio group, a 2-pyridylthio group, and a 1-phenyltetrazolylthio group);
an alkylsulfinyl group (an alkylsulfinyl group having preferably 1 to 32 carbon atoms and more preferably 1 to 24 carbon atoms, such as a dodecanesulfinyl group);
an arylsulfinyl group (an arylsulfinyl group having preferably 6 to 32 carbon atoms and more preferably 6 to 24 carbon atoms, such as a phenylsulfinyl group);
an alkylsulfonyl group (an alkylsulfonyl group having preferably 1 to 48 carbon atoms and more preferably 1 to 24 carbon atoms, such as a methylsulfonyl group, an ethylsulfonyl group, a propylsulfonyl group, a butylsulfonyl group, an isopropylsulfonyl group, a 2-ethylhexylsulfonyl group, a hexadecylsulfonyl group, an octylsulfonyl group, and a cyclohexylsulfonyl group);
an arylsulfonyl group (an arylsulfonyl group having preferably 6 to 48 carbon atoms and more preferably 6 to 24 carbon atoms, such as a phenylsulfonyl group and a 1-naphthylsulfonyl group);
a sulfamoyl group (a sulfamoyl group having preferably 32 or less carbon atoms and more preferably 24 or less carbon atoms, such as a sulfamoyl group, an N,N-dipropylsulfamoyl group, an N-ethyl-N-dodecylsulfamoyl group, an N-ethyl-N-phenylsulfamoyl group, an N-cyclohexylsulfamoyl group, and an N-(2-ethylhexyl) sulfamoyl group);
a phosphonyl group (a phosphonyl group having preferably 1 to 32 carbon atoms and more preferably 1 to 24 carbon atoms, such as a phenoxyphosphonyl group, an octyloxyphosphonyl group, and a phenylphosphonyl group);
a phosphinoylamino group (a phosphinoylamino group having preferably 1 to 32 carbon atoms and more preferably 1 to 24 carbon atoms, such as a diethoxyphosphinoylamino group and a dioctyloxyphosphinoylamino group);
epoxy group; —NHCOCH₃; —SO₂NHC₂H₄OCH₃; and —NHSO₂CH₃, in which two or more thereof may be combined.

These substituents may be further substituted with these substituents. In addition, in a case of having two or more of the substituents, the substituents may be the same or different from each other. Furthermore, if possible, these substituents may be bonded to each other to form a ring.

### [Liquid Crystal Composition]

The liquid crystal composition according to the embodiment of the present invention is a liquid crystal composition containing a compound represented by Formula (A) described later (hereinafter, also abbreviated as “specific compound A”), and a liquid crystal compound.

In the present invention, as described above, in a case where the liquid crystal composition containing the specific compound A and the liquid crystal compound is used, precipitation is suppressed and aligning properties are also favorable.

The reason for this is not clear, but the present inventors presume as follows.

That is, it is presumed that, in a case where the specific compound A has a predetermined functional group such as an alkoxy group as X¹ in Formula (A) described later, it is possible to suppress the precipitation (for example, crystallization of the liquid crystal compound) without hindering the alignment of the liquid crystal compound, as compared with a case where the specific compound A has a functional group containing an active hydrogen (for example, a hydroxyl group).

Hereinafter, the specific compound A and the liquid crystal compound contained in the liquid crystal composition according to the embodiment of the present invention will be described in detail.

### Specific Compound A

The specific compound A is a compound represented by Formula (A).

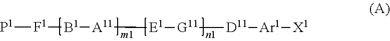

In Formula (A), P¹ represents a polymerizable group.

Here, the polymerizable group is not particularly limited, but is preferably a radically polymerizable group or a cationically polymerizable group.

A known radically polymerizable group can be used as the radically polymerizable group, and suitable examples thereof include an acryloyloxy group or a methacryloyloxy group. In this case, it is known that the acryloyloxy group generally has a high polymerization rate, and from the viewpoint of improving productivity, the acryloyloxy group is preferable. However, the methacryloyloxy group can also be used as the polymerizable group.

A known cationically polymerizable group can be used as the cationically polymerizable group, and specific examples thereof include an alicyclic ether group, a cyclic acetal group, a cyclic lactone group, a cyclic thioether group, a spiro orthoester group, and a vinyloxy group. Among these, an alicyclic ether group or a vinyloxy group is suitable, and an epoxy group, an oxetanyl group, or a vinyloxy group is particularly preferable.

Examples of a particularly preferred polymerizable group include a polymerizable group represented by any one of Formulae (P-1) to (P-20).

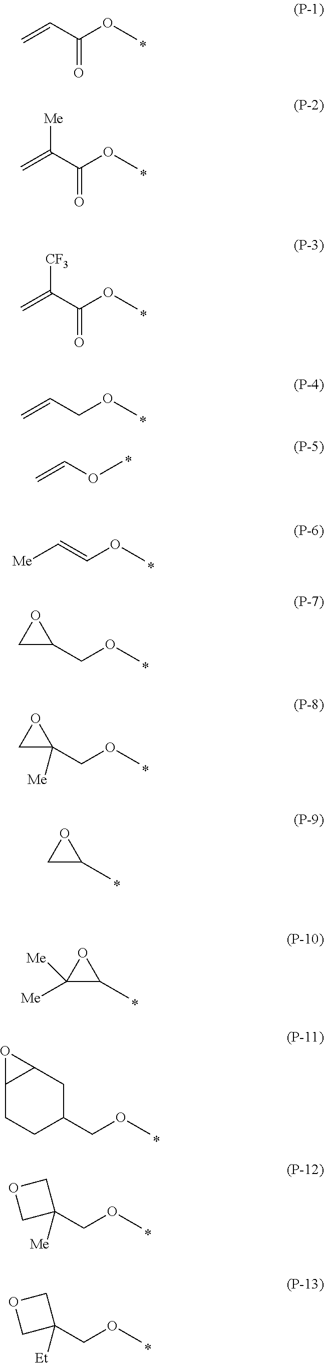

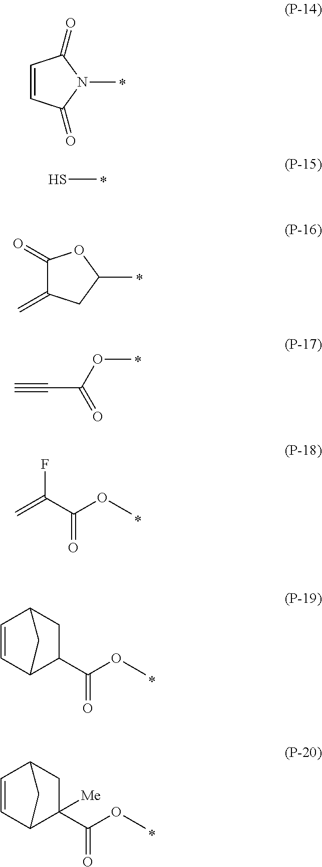

In Formula (A), F¹ represents a spacer group.

Here, examples of the spacer group include a divalent aliphatic hydrocarbon group having 1 to 20 carbon atoms. Here, one or more of —CH₂—'s constituting the aliphatic hydrocarbon group may be replaced with —O—, —S—, —NH—, —N(Q)-, or —CO—. Q represents a substituent. In addition, hydrogen atoms contained in the aliphatic hydrocarbon group may be each independently replaced with a halogen atom, an alkyl group, or an alkoxy group.

In addition, examples of the divalent aliphatic hydrocarbon group having 1 to 20 carbon atoms include a linear or branched alkylene group having 1 to 20 carbon atoms, a linear or branched alkenylene group having 1 to 20 carbon atoms, and a linear or branched alkynylene group having 1 to 20 carbon atoms.

As the linear or branched alkylene group having 1 to 20 carbon atoms, an alkylene group having 1 to 12 carbon atoms is preferable and an alkylene group having 1 to 10 carbon atoms is more preferable; and suitable examples thereof include a methylene group, an ethylene group, a propylene group, a butylene group, a pentylene group, and a hexylene group.

As the linear or branched alkenylene group having 1 to 20 carbon atoms, an alkenylene group having 2 to 10 carbon atoms is preferable and an alkenylene group having 2 to 4 carbon atoms is more preferable; and suitable examples thereof include an ethenylene group.

As the linear or branched alkynylene group having 1 to 20 carbon atoms, an alkynylene group having 2 to 10 carbon atoms is preferable and an alkynylene group having 2 to 4 carbon atoms is more preferable; and suitable examples thereof include an ethynylene group.

As described above, one or more of —CH₂—'s constituting the aliphatic hydrocarbon group may be replaced with —O—, —S—, —NH—, —N(Q)-, or —CO—; and examples of the substituent represented by Q include the substituents described in the substituent group A above. Among these, an alkyl group, an alkoxy group, an alkoxycarbonyl group, an alkylcarbonyloxy group, or a halogen atom is preferable.

In Formula (A), B¹, E¹, and D¹¹ each independently represent a single bond or a divalent linking group.

Here, examples of the divalent linking group include —CO—, —O—, —S—, —C(═S)—, —CR¹R²—, —CR³—CR⁴—, —NR⁵—, or a divalent linking group consisting of a combination of two or more of these groups. R¹ to R⁵ each independently represent a hydrogen atom, a fluorine atom, or an alkyl group having 1 to 12 carbon atoms.

Specific examples of the divalent linking group include —CO—, —O—, —CO—O—, —C(═S)O—, —CR¹R²—, —CR¹R²—CR¹R²—, —O—CR¹R²—, —CR¹R²—O—CR¹R²—, —CO—O—CR¹R²—, —O—CO—CR¹R²—, —CR¹R²—O—CO—CR¹R²—, —CR¹R²—CO—O—CR¹R²—, —NR⁵—CR¹R²—, and —CO—NR⁵—. R¹, R², and R⁵ each independently represent a hydrogen atom, a fluorine atom, or an alkyl group having 1 to 12 carbon atoms.

Among these, any of —CO—, —O—, or —CO—O— is preferable.

In Formula (A), A¹¹ and G¹¹ each independently represent a divalent aromatic hydrocarbon group having 6 to 20 carbon atoms or a divalent alicyclic hydrocarbon group having 3 to 16 carbon atoms.

Here, the number of carbon atoms in the divalent aromatic hydrocarbon group is preferably 6 to 18, more preferably 6 to 16, still more preferably 6 to 10, and particularly preferably 6.

In addition, the number of carbon atoms in the divalent alicyclic hydrocarbon group is preferably 4 to 15, more preferably 5 to 10, and still more preferably 5 or 6.

The hydrogen atoms in the divalent aromatic hydrocarbon group or the divalent alicyclic hydrocarbon group may be each independently replaced with a halogen atom (for example, a fluorine atom, a chlorine atom, a bromine atom, an iodine atom, or the like), —R^{L1}, —OR^{L1}, a cyano group, or a nitro group. R^{L1} represents an alkyl group having 1 to 4 carbon atoms, and hydrogen atoms in the alkyl group may be each independently replaced with a fluorine atom.

Here, examples of the alkyl group having 1 to 4 carbon atoms as R^{L1} include a methyl group, an ethyl group, a propyl group, an isopropyl group, a butyl group, an isobutyl group, a sec-butyl group, and a tert-butyl group; and an alkyl group having 1 to 3 carbon atoms is preferable, an alkyl group having 1 or 2 carbon atoms is more preferable, and a methyl group is still more preferable.

In addition, examples of an alkoxy group having 1 to 4 carbon atoms as —OR^{L1} include a methoxy group, an ethoxy group, a propoxy group, an isopropoxy group, a butoxy group, an isobutoxy group, a sec-butoxy group, and a tert-butoxy group; and an alkoxy group having 1 to 3 carbon atoms is preferable, an alkoxy group having 1 or 2 carbon atoms is more preferable, and a methoxy group is still more preferable.

Carbon atoms in the divalent aromatic hydrocarbon group or the divalent alicyclic hydrocarbon group may be each independently replaced with an oxygen atom, a sulfur atom, or a nitrogen atom. One carbon atom may be replaced with an oxygen atom, a sulfur atom, or a nitrogen atom, or two or more carbon atoms may be replaced with two or more atoms selected from the group consisting of an oxygen atom, a sulfur atom, and a nitrogen atom. For example, —CH═ in the divalent aromatic hydrocarbon group may be replaced with —N═. In addition, —CH₂—'s (methylene group) in the divalent alicyclic hydrocarbon group may be each independently replaced with —O—, —S—, —NH—, or —NR^{L1}—; and —CH(—)—'s in the alicyclic hydrocarbon group may be each independently replaced with —N(—)—. Here, R^{L1} has the same meaning as described above.

Examples of the divalent aromatic hydrocarbon group include groups represented by Formulae (a-1) to (a-8). As the divalent aromatic hydrocarbon group, a 1,4-phenylene group is preferable.

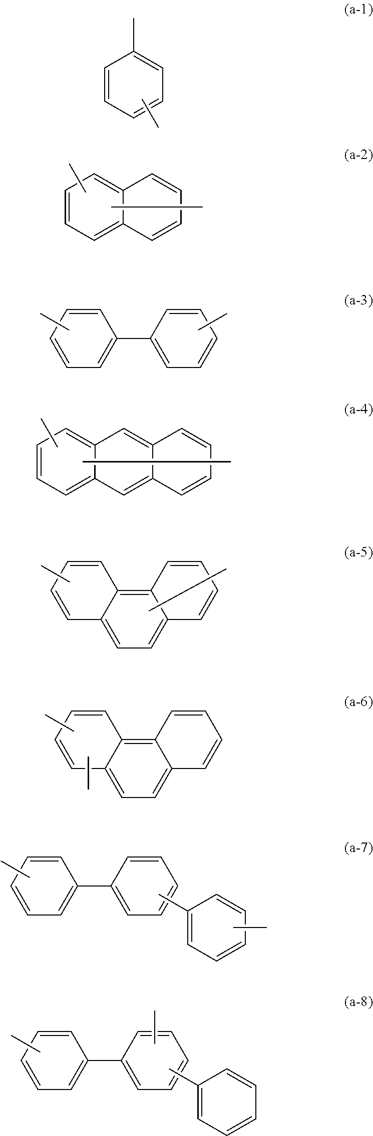

Examples of the divalent alicyclic hydrocarbon group include groups represented by Formulae (g-1) to (g-4). Examples of a divalent alicyclic hydrocarbon group in which —CH₂-in the alicyclic hydrocarbon group is replaced with —O—, —S—, —NH—, or —NR^{L1}— include groups represented by Formulae (g-5) to (g-8). Examples of a divalent alicyclic hydrocarbon group in which —CH(—)— in the alicyclic hydrocarbon group is replaced with —N(—)— include groups represented by Formulae (g-9) and (g-10). These divalent alicyclic hydrocarbon groups are preferably an alicyclic hydrocarbon group of a 5-membered ring or a 6-membered ring.

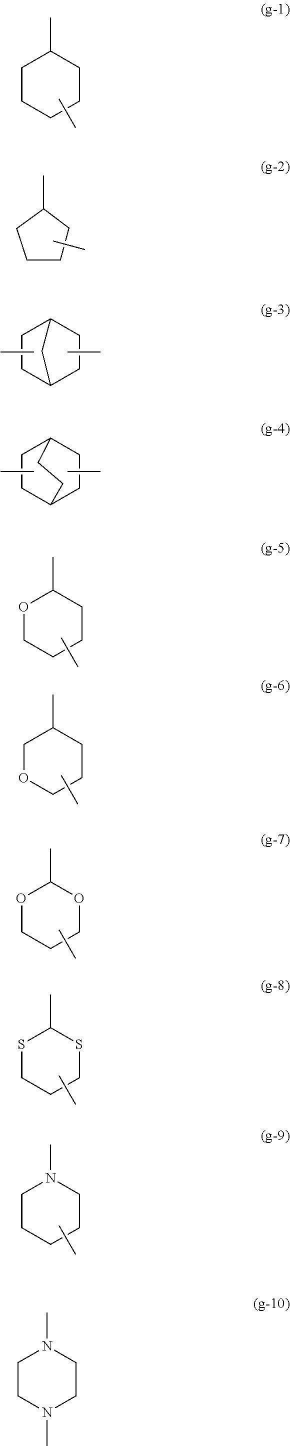

From the viewpoint of producing the compound according to the present invention, the divalent alicyclic hydrocarbon group is preferably a cycloalkane diyl group represented by Formula (g-1), more preferably a cyclohexane-1,4-diyl group, and still more preferably a trans-cyclohexane-1,4-diyl group.

In the present invention, from the viewpoint of producing the specific compound A, A¹¹ is preferably a divalent aromatic hydrocarbon group.

In addition, from the viewpoint of compatibility with the liquid crystal compound, G¹¹ is preferably a divalent alicyclic hydrocarbon group, more preferably a cyclohexane-1,4-diyl group, and still more preferably a trans-cyclohexane-1,4-diyl group.

In Formula (A), m1 and n1 each independently represent an integer of 0 to 3. In a case where m1 is 2 or 3, a plurality of B1's may be the same or different from each other and a plurality of A¹¹'s may be the same or different from each other. In a case where n1 is 2 or 3, a plurality of E¹'s may be the same or different from each other and a plurality of G¹¹'s may be the same or different from each other.

Here, both m1 and n1 may be 0, but it is preferable that, in a case where any one of m1 or n1 is 0, the other represents an integer of 2 or 3.

In addition, both m1 and n1 are preferably 1 or 2, and more preferably 1.

In Formula (A), X¹ represents —OR^{L2}, —O—(C—O)—R^{L3}, —O—(C═O)—OR^{L4}, —O—(C—O)—NH—R^{L5}, —O—(C═O)—NR^{L6}—R^{L7}, or —O—(C═S)—R^{L8}.

R^{L2} represents an alkyl group having 1 to 4 carbon atoms, an acryloyl group, or a methacryloyl group, R^{L3} represents an alkyl group having 1 to 4 carbon atoms, and R^{L4} to R^{L8} each independently represent a non-cyclic alkyl group, a monovalent aromatic group, or a monovalent alicyclic group.

Here, examples of the alkyl group having 1 to 4 carbon atoms include a methyl group, an ethyl group, a propyl group, an isopropyl group, a butyl group, an isobutyl group, a sec-butyl group, and a tert-butyl group; and an alkyl group having 1 to 3 carbon atoms is preferable, an alkyl group having 1 or 2 carbon atoms is more preferable, and a methyl group is still more preferable.

In addition, examples of the non-cyclic alkyl group include an alkyl group having 1 to 6 carbon atoms; and specific examples thereof include a methyl group, an ethyl group, a propyl group, an isopropyl group, a n-butyl group, an isobutyl group, a sec-butyl group, a tert-butyl group, a n-pentyl group, and a n-hexyl group. It is preferable that the non-cyclic alkyl group does not have a substituent.

Examples of the monovalent aromatic group include an aromatic hydrocarbon group having 6 to 12 carbon atoms and an aromatic heterocyclic group having 3 to 12 carbon atoms; and specific examples thereof include an aryl group such as a phenyl group, a 2,6-diethylphenyl group, and a naphthyl group, and a heteroaryl group such as a thienyl group, a thiazolyl group, a furyl group, and a pyridyl group. It is preferable that the monovalent aromatic group does not have a substituent.

Examples of the monovalent alicyclic group include an alicyclic hydrocarbon group having 6 to 20 carbon atoms; and specific examples thereof include a cyclohexylene group, a cyclopentylene group, a norbornylene group, and an adamantylene group. It is preferable that the monovalent alicyclic group does not have a substituent.

In the present invention, from the viewpoint of further suppressing the precipitation and further improving the aligning properties, X¹ is preferably —OR^{L2} or —O—(C═O)—R^{L3}, and more preferably a methoxy group (—OCH₃), an ethoxy group (—OCH₂CH₃), a butoxy group (—OCH₂CH₂CH₂CH₃), or an acetoxy group (—O—C(═O)—CH₃).

In Formula (A), Ar¹ represents any aromatic ring selected from the group consisting of groups represented by Formulae (Ar-1) to (Ar-3). In Formulae (Ar-1) to (Ar-3), * represents a bonding position to D¹¹ or X¹ in Formula (A) described above.

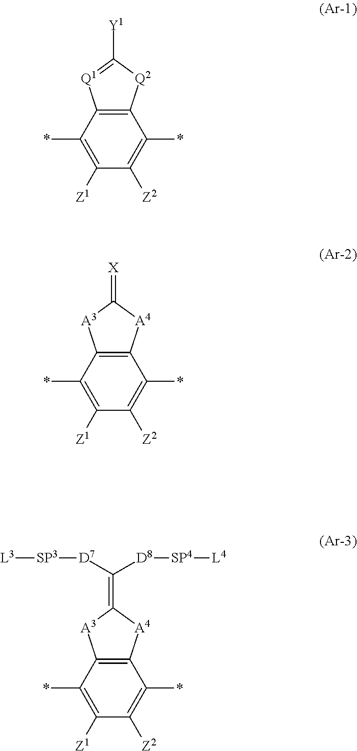

In Formula (Ar-1), Q¹ represents N or CH, Q² represents —S—, —O—, or —N(R⁶)—, where R⁶ represents a hydrogen atom or an alkyl group having 1 to 6 carbon atoms, Y¹ represents an aromatic hydrocarbon group having 6 to 12 carbon atoms, which may have a substituent, an aromatic heterocyclic group having 3 to 12 carbon atoms, which may have a substituent, or an alicyclic hydrocarbon group having 6 to 20 carbon atoms, which may have a substituent, and one or more of —CH₂—'s constituting the alicyclic hydrocarbon group may be replaced with —O—, —S—, or —NH—.

Here, specific examples of the alkyl group having 1 to 6 carbon atoms represented by one aspect of R⁶ include a methyl group, an ethyl group, a propyl group, an isopropyl group, a n-butyl group, an isobutyl group, a sec-butyl group, a tert-butyl group, a n-pentyl group, and a n-hexyl group.

Examples of the aromatic hydrocarbon group having 6 to 12 carbon atoms, represented by one aspect of Y¹, include aryl groups such as a phenyl group, a 2,6-diethylphenyl group, and a naphthyl group.

Examples of the aromatic heterocyclic group having 3 to 12 carbon atoms, represented by one aspect of Y¹, include a heteroaryl group such as a thienyl group, a thiazolyl group, a furyl group, and a pyridyl group; and a group obtained by removing one hydrogen atom from any of an indole ring, a benzofuran ring, a benzothiophene ring, a benzimidazole ring, a benzothiazole ring, or a benzoxazole ring. Among these, as the monovalent aromatic heterocyclic group having 3 to 12 carbon atoms, represented by one aspect of Y¹, a group obtained by removing one hydrogen atom from a benzofuran ring or a benzothiazole ring is preferable.

Examples of the alicyclic hydrocarbon group having 6 to 20 carbon atoms, represented by one aspect of Y¹, include a cyclohexylene group, a cyclopentylene group, a norbornylene group, and an adamantylene group.

In addition, examples of the substituent which may be included in Y¹ include the substituents described in the substituent group A above; and among these, an alkyl group, an alkoxy group, an alkoxycarbonyl group, an alkylcarbonyloxy group, a nitro group, a cyano group, or a halogen atom is preferable.

In addition, in Formulae (Ar-1) to (Ar-3), Z¹ and Z² each independently represent a hydrogen atom, a monovalent aliphatic hydrocarbon group having 1 to 20 carbon atoms, a monovalent alicyclic hydrocarbon group having 3 to 20 carbon atoms, a monovalent aromatic hydrocarbon group having 6 to 20 carbon atoms, a monovalent aromatic heterocyclic group having 6 to 20 carbon atoms, a halogen atom, a cyano group, a nitro group, —OR⁷, —NR⁸R⁹, —SR¹⁰, —COOR¹¹, or —COR¹², where R⁷ to R¹² each independently represent a hydrogen atom or an alkyl group having 1 to 6 carbon atoms and Z¹ and Z² may be bonded to each other to form an aromatic ring.

As the monovalent aliphatic hydrocarbon group having 1 to 20 carbon atoms, an alkyl group having 1 to 15 carbon atoms is preferable, an alkyl group having 1 to 8 carbon atoms is more preferable; and specifically, a methyl group, an ethyl group, an isopropyl group, a tert-pentyl group (1,1-dimethylpropyl group), a tert-butyl group, or 1,1-dimethyl-3,3-dimethyl-butyl group is still more preferable and a methyl group, an ethyl group, or a tert-butyl group is particularly preferable.

Examples of the monovalent alicyclic hydrocarbon group having 3 to 20 carbon atoms include monocyclic saturated hydrocarbon groups such as a cyclopropyl group, a cyclobutyl group, a cyclopentyl group, a cyclohexyl group, a cycloheptyl group, a cyclooctyl group, a cyclodecyl group, a methylcyclohexyl group, and an ethylcyclohexyl group; monocyclic unsaturated hydrocarbon groups such as a cyclobutenyl group, a cyclopentenyl group, a cyclohexenyl group, a cycloheptenyl group, a cyclooctenyl group, a cyclodecenyl group, a cyclopentadienyl group, a cyclohexadienyl group, a cyclooctadienyl group, and a cyclodecadiene group; and polycyclic saturated hydrocarbon groups such as a bicyclo[2.2.1]heptyl group, a bicyclo[2.2.2]octyl group, a tricyclo[5.2.1.0^{2,6}]decyl group, a tricyclo[3.3.1.1^{3,7}]decyl group, a tetracyclo[6.2.1.1^{3,6}.0^{2,7}]dodecyl group, and an adamantyl group.

Specific examples of the monovalent aromatic hydrocarbon group having 6 to 20 carbon atoms include a phenyl group, a 2,6-diethylphenyl group, a naphthyl group, and a biphenyl group, and an aryl group having 6 to 12 carbon atoms (particularly, a phenyl group) is preferable.

Specific examples of the monovalent aromatic heterocyclic group having 6 to 20 carbon atoms include a 4-pyridyl group, a 2-furyl group, a 2-thienyl group, a 2-pyrimidinyl group, and a 2-benzothiazolyl group.

Examples of the halogen atom include a fluorine atom, a chlorine atom, a bromine atom, and an iodine atom, and among these, a fluorine atom, a chlorine atom, or a bromine atom is preferable.

On the other hand, specific examples of the alkyl group having 1 to 6 carbon atoms represented by one aspect of R⁷ to R¹⁰ include a methyl group, an ethyl group, a propyl group, an isopropyl group, a n-butyl group, an isobutyl group, a sec-butyl group, a tert-butyl group, a n-pentyl group, and a n-hexyl group.

As described above, Z¹ and Z² may be bonded to each other to form an aromatic ring, and examples of a structure in a case where Z¹ and Z² in Formula (Ar-1) are bonded to each other to form an aromatic ring include a group represented by Formula (Ar-1a). In Formula (Ar-1a), * represents a bonding position to D¹¹ or X¹ in Formula (A) described above.

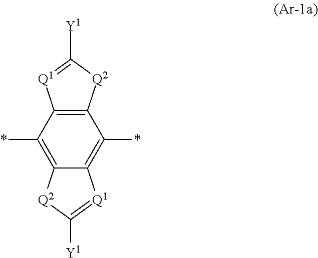

Here, in Formula (Ar-1a), examples of Q¹, Q², and Y¹ include the same as those described in Formula (Ar-1) above.

In addition, in Formulae (Ar-2) and (Ar-3), A³ and A⁴ each independently represent a group selected from the group consisting of —O—, —N(R¹³)—, —S—, and —CO—, and R¹³ represents a hydrogen atom or a substituent.

Examples of the substituent represented by one aspect of R¹³ include the substituents described in the substituent group A above; and among these, an alkyl group, an alkoxy group, an alkoxycarbonyl group, an alkylcarbonyloxy group, or a halogen atom is preferable.

In addition, in Formula (Ar-2), X represents a non-metal atom of Group 14 to Group 16. Here, a hydrogen atom or a substituent may be bonded to the non-metal atom.

In addition, examples of the non-metal atom of Groups 14 to 16, represented by X, include an oxygen atom, a sulfur atom, a nitrogen atom to which a hydrogen atom or a substituent is bonded [═N—R^{N1}, R^{N1} represents a hydrogen atom or a substituent], and a carbon atom to which a hydrogen atom or a substituent is bonded [—C—(R^{C1})₂; R^{C1} represents a hydrogen atom or a substituent].

Examples of the substituent include the substituents described in the substituent group A above; and among these, suitable examples thereof include an alkyl group, an alkoxy group, an alkyl-substituted alkoxy group, a cyclic alkyl group, an aryl group (for example, a phenyl group, a naphthyl group, or the like), a cyano group, an amino group, a nitro group, an alkylcarbonyl group, a sulfo group, and a hydroxyl group.

In addition, in Formula (Ar-3), D⁷ and D⁸ each independently represent a single bond, —CO—, —O—, —S—, —C(═S)—, —CR¹R²—, —CR³—CR⁴—, —NR⁵—, or a divalent linking group consisting of a combination of two or more of these groups, where R¹ to R⁵ each independently represent a hydrogen atom, a fluorine atom, or an alkyl group having 1 to 12 carbon atoms.

Here, examples of the divalent linking group include the same as those described for B¹, E¹, and D¹¹ in Formula (A) above.

In addition, in Formula (Ar-3), SP³ and SP⁴ each independently represent a single bond or a divalent aliphatic hydrocarbon group having 1 to 20 carbon atoms. Here, one or more of —CH₂—'s constituting the aliphatic hydrocarbon group may be replaced with —O—, —S—, —NH—, —N(Q)-, or —CO—. Q represents a substituent. Examples of the substituent represented by Q include the substituents described in the substituent group A above; and among these, an alkyl group, an alkoxy group, an alkoxycarbonyl group, an alkylcarbonyloxy group, or a halogen atom is preferable.

Here, examples of the divalent aliphatic hydrocarbon group include the same as those described for F¹ in Formula (A) above.

In addition, in Formula (Ar-3), L³ and L⁴ each independently represent a monovalent organic group.

Here, examples of the monovalent organic group include the substituents described in the substituent group A above; and suitable examples thereof include an alkyl group, an aryl group, a heteroaryl group, an alkoxy group, a cyano group, and a carboxy group.

The alkyl group may be linear, branched, or cyclic, but is preferably linear. The number of carbon atoms in the alkyl group is preferably 1 to 30, more preferably 1 to 20, and still more preferably 1 to 10.

In addition, the aryl group may be monocyclic or polycyclic, but is preferably monocyclic. The number of carbon atoms in the aryl group is preferably 6 to 25 and more preferably 6 to 10.

In addition, the heteroaryl group may be monocyclic or polycyclic. The number of heteroatoms constituting the heteroaryl group is preferably 1 to 3. The heteroatom constituting the heteroaryl group is preferably a nitrogen atom, a sulfur atom, or an oxygen atom. The number of carbon atoms in the heteroaryl group is preferably 6 to 18 and more preferably 6 to 12.

In addition, the alkyl group, the aryl group, and the heteroaryl group may be unsubstituted or may have a substituent. Examples of the substituent include the substituents described in the substituent group A above; and among these, an alkyl group, an alkoxy group, an alkoxycarbonyl group, an alkylcarbonyloxy group, or a halogen atom is preferable.

In addition, the monovalent organic group may be a polymerizable group, and suitable examples thereof include the polymerizable group represented by any of Formulae (P-1) to (P-20) described above.

In the present invention, from the viewpoint of satisfying desired optical characteristics, Ar¹ in Formula (A) is preferably the aromatic ring represented by Formula (Ar-1) or (Ar-2) described above.

Suitable examples of the specific compound A include compounds represented by Formulae (1) to (17), and specific examples thereof include compounds in which K (side chain structure) in Formulae (1) to (17) has side chain structures shown in Tables 1 to 3, and X¹ in Formulae (1) to (17) is a methoxy group (—OCH₃), an ethoxy group (—OCH₂CH₃), a butoxy group (—OCH₂CH₂CH₂CH₃), or an acetoxy group (—O—C(═O)—CH₃), and compounds in which K and X¹ in Formulae (2), (3), (5) to (8), (11), (13) to (17) are interchanged.

In Tables 1 to 3 below, “*” shown in the side chain structure of K represents a bonding position to an aromatic ring.

In addition, in the side chain structures shown as 2-2 in Table 2 and 3-2 in Table 3, a group adjacent to each of the acryloyloxy group and the methacryloyl group represents a propylene group (a group in which a methyl group is replaced with an ethylene group), and represents a mixture of regioisomers in which the position of the methyl group is different.

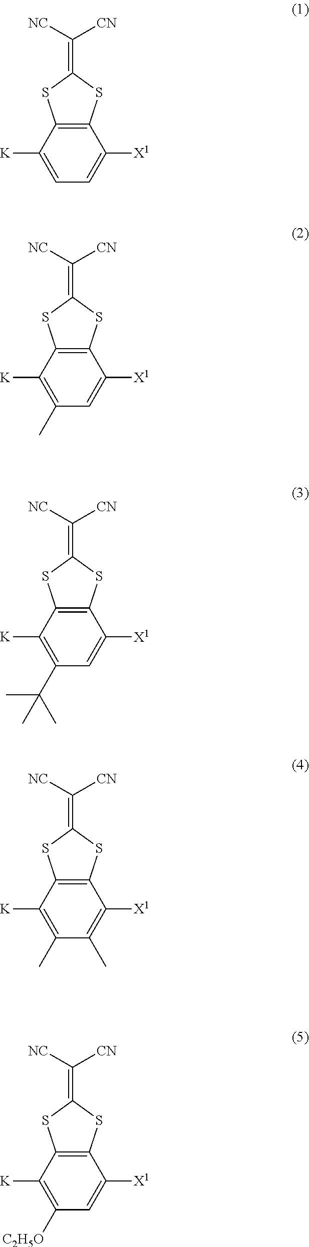

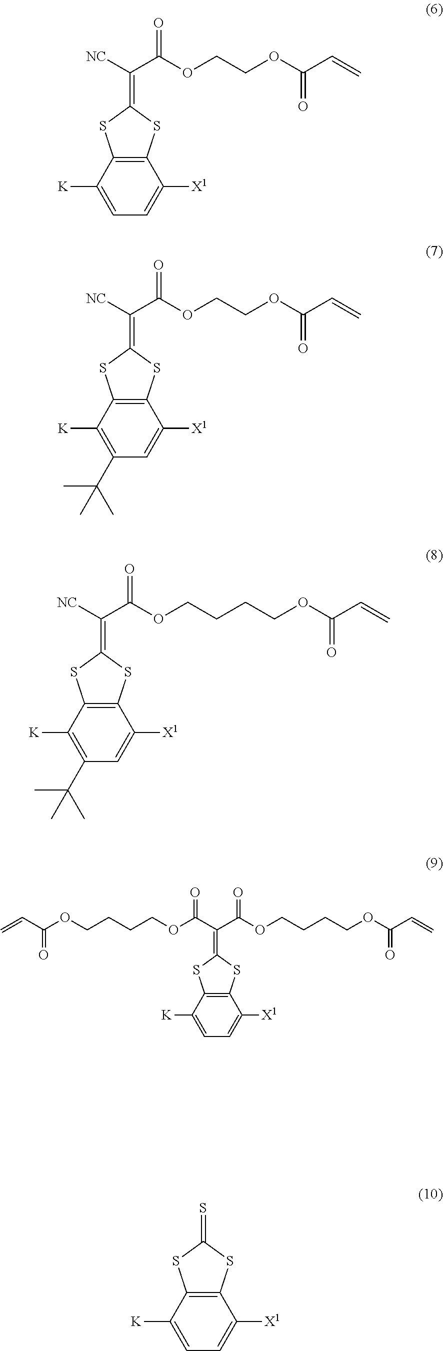

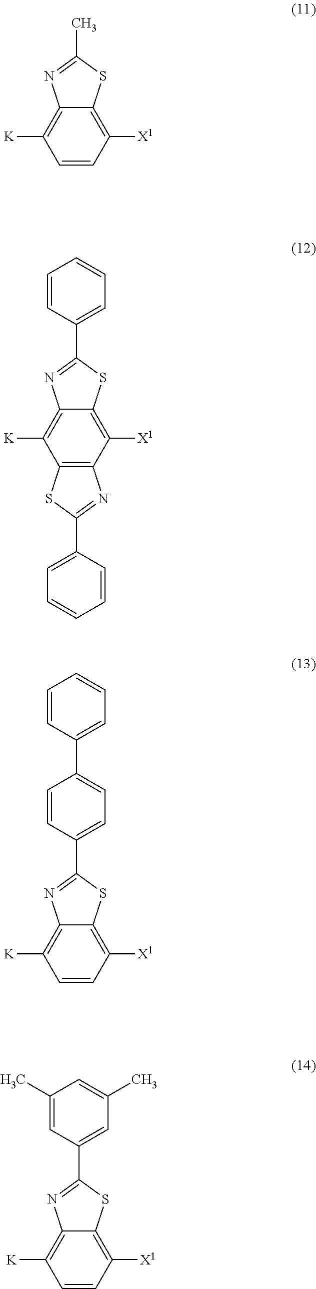

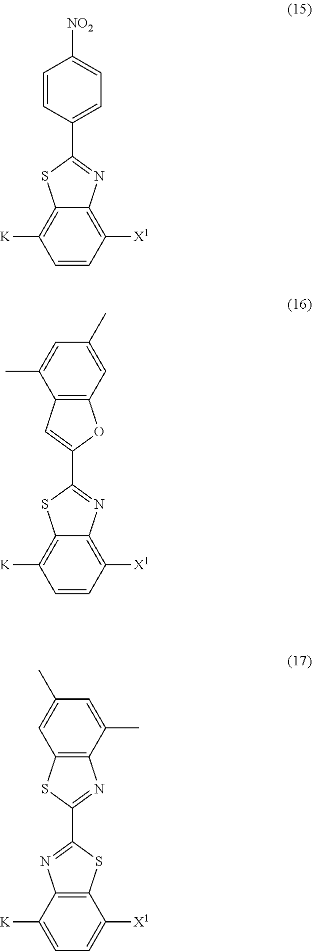

TABLE 1

| | K (side chain structure) |
|---|---|
| 1-1 | 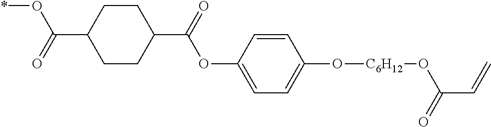 |
| 1-2 | 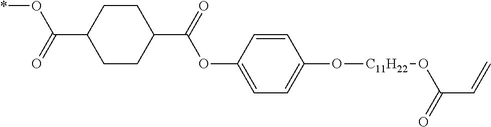 |
| 1-3 | 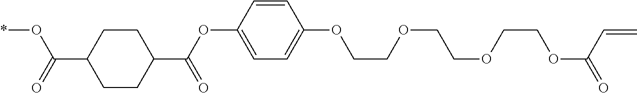 [SMILES: *OC(=O)C1CCC(C(=O)Oc2ccc(OCCOCCOCCOC(=O)C=C)cc2)CC1] |
| 1-4 | 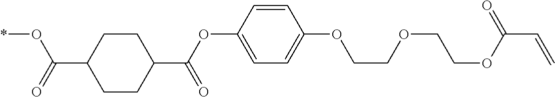 [SMILES: *OC(=O)C1CCC(C(=O)Oc2ccc(OCCOCCOC(=O)C=C)cc2)CC1] |
| 1-5 | 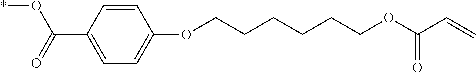 [SMILES: *OC(=O)c1ccc(OCCCCCCOC(=O)C=C)cc1] |
| 1-6 | 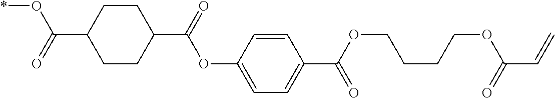 [SMILES: *OC(=O)C1CCC(C(=O)Oc2ccc(C(=O)OCCCCOC(=O)C=C)cc2)CC1] |
| 1-7 | 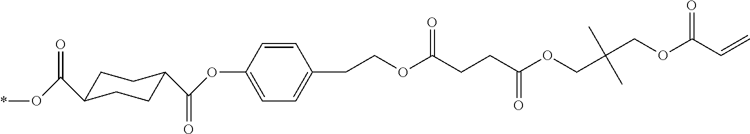 [SMILES: *OC(=O)C1CCC(C(=O)Oc2ccc(CCOC(=O)CCC(=O)OCC(C)(C)COC(=O)C=C)cc2)CC1] |

TABLE 2

| | K (side chain structure) |
|---|---|
| 2-1 | 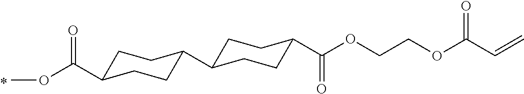 [SMILES: C=CC(=O)OCCOC(=O)C1CCC(C2CCC(C(=O)OC)CC2)CC1] |
| 2-2 | 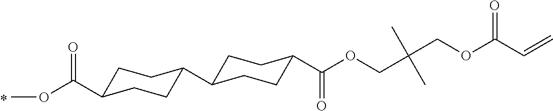 [SMILES: C=CC(=O)OCC(C)(C)COC(=O)C1CCC(C2CCC(C(=O)OC)CC2)CC1] |
| 2-3 | 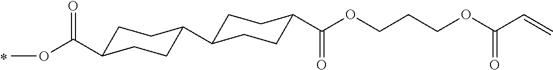 [SMILES: C=CC(=O)OCCCOC(=O)C1CCC(C2CCC(C(=O)OC)CC2)CC1] |
| 2-4 | 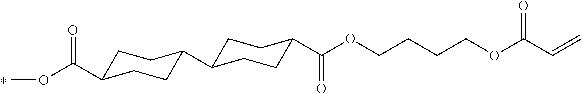 [SMILES: C=CC(=O)OCCCCOC(=O)C1CCC(C2CCC(C(=O)OC)CC2)CC1] |
| 2-5 | 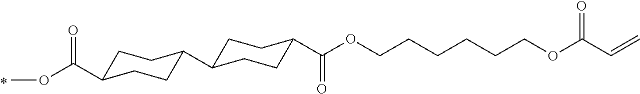 [SMILES: C=CC(=O)OCCCCCCOC(=O)C1CCC(C2CCC(C(=O)OC)CC2)CC1] |
| 2-6 | 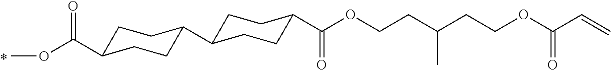 [SMILES: C=CC(=O)OCCC(C)CCOC(=O)C1CCC(C2CCC(C(=O)OC)CC2)CC1] |
| 2-7 | 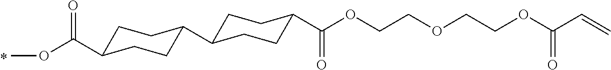 [SMILES: C=CC(=O)OCCOCCOC(=O)C1CCC(C2CCC(C(=O)OC)CC2)CC1] |
| 2-8 | 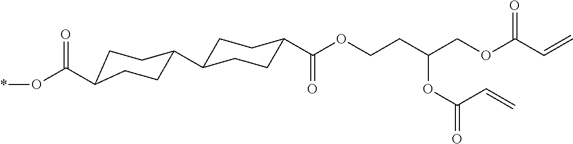 [SMILES: *OC(=O)C1CCC(C2CCC(C(=O)OCCC(COC(=O)C=C)OC(=O)C=C)CC2)CC1] |
| 2-9 | 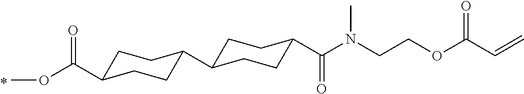 [SMILES: C=CC(=O)OCCN(C)C(=O)C1CCC(C2CCC(C(=O)OC)CC2)CC1] |
| 2-10 | 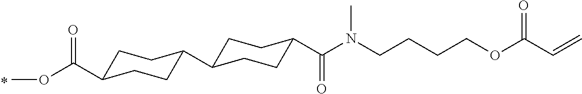 [SMILES: C=CC(=O)OCCCCN(C)C(=O)C1CCC(C2CCC(C(=O)OC)CC2)CC1] |
| 2-41 | 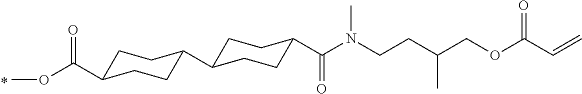 [SMILES: C=CC(=O)OCC(C)CCN(C)C(=O)C1CCC(C2CCC(C(=O)OC)CC2)CC1] |
| 2-12 | 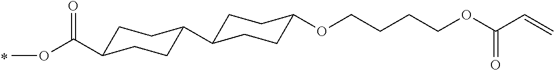 [SMILES: C=CC(=O)OCCCCOC1CCC(C2CCC(C(=O)OC)CC2)CC1] |
| 2-13 | 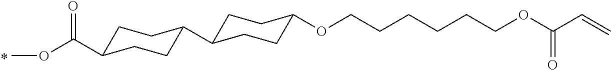 [SMILES: C=CC(=O)OCCCCCCOC1CCC(C2CCC(C(=O)OC)CC2)CC1] |
| 2-14 | 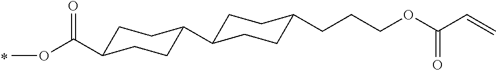 [SMILES: C=CC(=O)OCCCC1CCC(C2CCC(C(=O)OC)CC2)CC1] |

TABLE 3

| | K (side chain structure) |
|---|---|
| 3-1 | 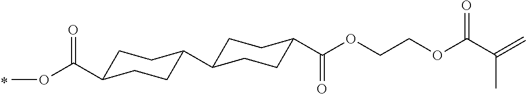 [SMILES: C=C(C)C(=O)OCCOC(=O)C1CCC(C2CCC(C(=O)OC)CC2)CC1] |
| 3-2 | 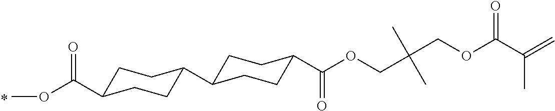 [SMILES: C=C(C)C(=O)OCC(C)(C)COC(=O)C1CCC(C2CCC(C(=O)OC)CC2)CC1] |
| 3-3 | 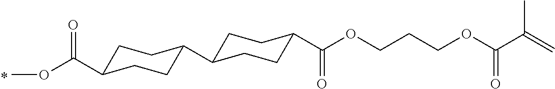 [SMILES: C=C(C)C(=O)OCCCOC(=O)C1CCC(C2CCC(C(=O)OC)CC2)CC1] |
| 3-4 | 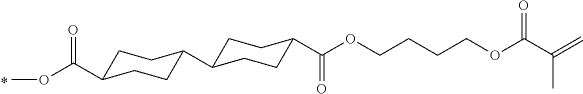 [SMILES: C=C(C)C(=O)OCCCCOC(=O)C1CCC(C2CCC(C(=O)OC)CC2)CC1] |
| 3-5 | 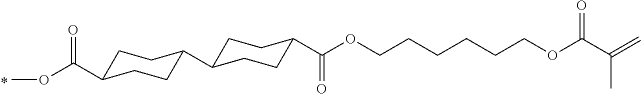 [SMILES: C=C(C)C(=O)OCCCCCCOC(=O)C1CCC(C2CCC(C(=O)OC)CC2)CC1] |
| 3-6 | 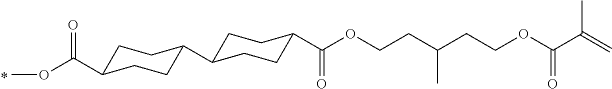 [SMILES: C=C(C)C(=O)OCCC(C)CCOC(=O)C1CCC(C2CCC(C(=O)OC)CC2)CC1] |
| 3-7 | 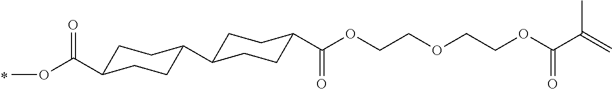 [SMILES: C=C(C)C(=O)OCCOCCOC(=O)C1CCC(C2CCC(C(=O)OC)CC2)CC1] |
| 3-8 | 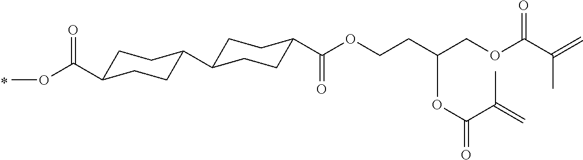 [SMILES: C=C(C)C(=O)OCC(CCOC(=O)C1CCC(C2CCC(C(=O)OC)CC2)CC1)OC(=O)C(=C)C] |
| 3-9 | 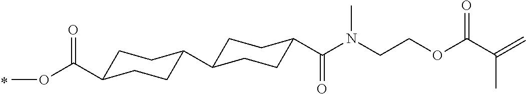 [SMILES: C=C(C)C(=O)OCCN(C)C(=O)C1CCC(C2CCC(C(=O)OC)CC2)CC1] |
| 3-10 | 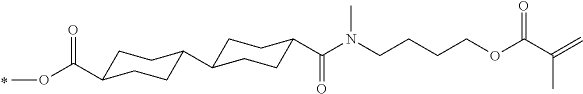 [SMILES: C=C(C)C(=O)OCCCCN(C)C(=O)C1CCC(C2CCC(C(=O)OC)CC2)CC1] |
| 3-11 | 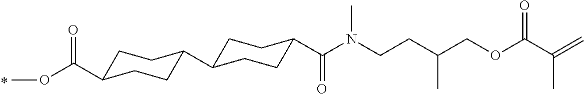 [SMILES: C=C(C)C(=O)OCC(C)CCN(C)C(=O)C1CCC(C2CCC(C(=O)OC)CC2)CC1] |
| 3-12 | 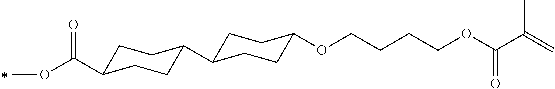 [SMILES: C=C(C)C(=O)OCCCCOC1CCC(C2CCC(C(=O)OC)CC2)CC1] |
| 3-13 | 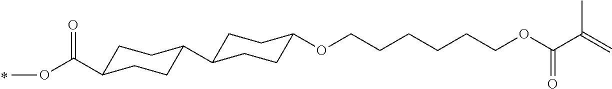 [SMILES: C=C(C)C(=O)OCCCCCCOC1CCC(C2CCC(C(=O)OC)CC2)CC1] |
| 3-14 | 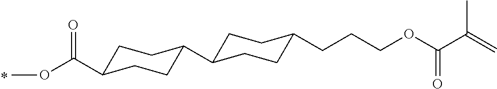 [SMILES: C=C(C)C(=O)OCCCC1CCC(C2CCC(C(=O)OC)CC2)CC1] |

Among these, specific compounds (A-1) to (A-24) represented by the following formulae are preferable as the specific compound A.

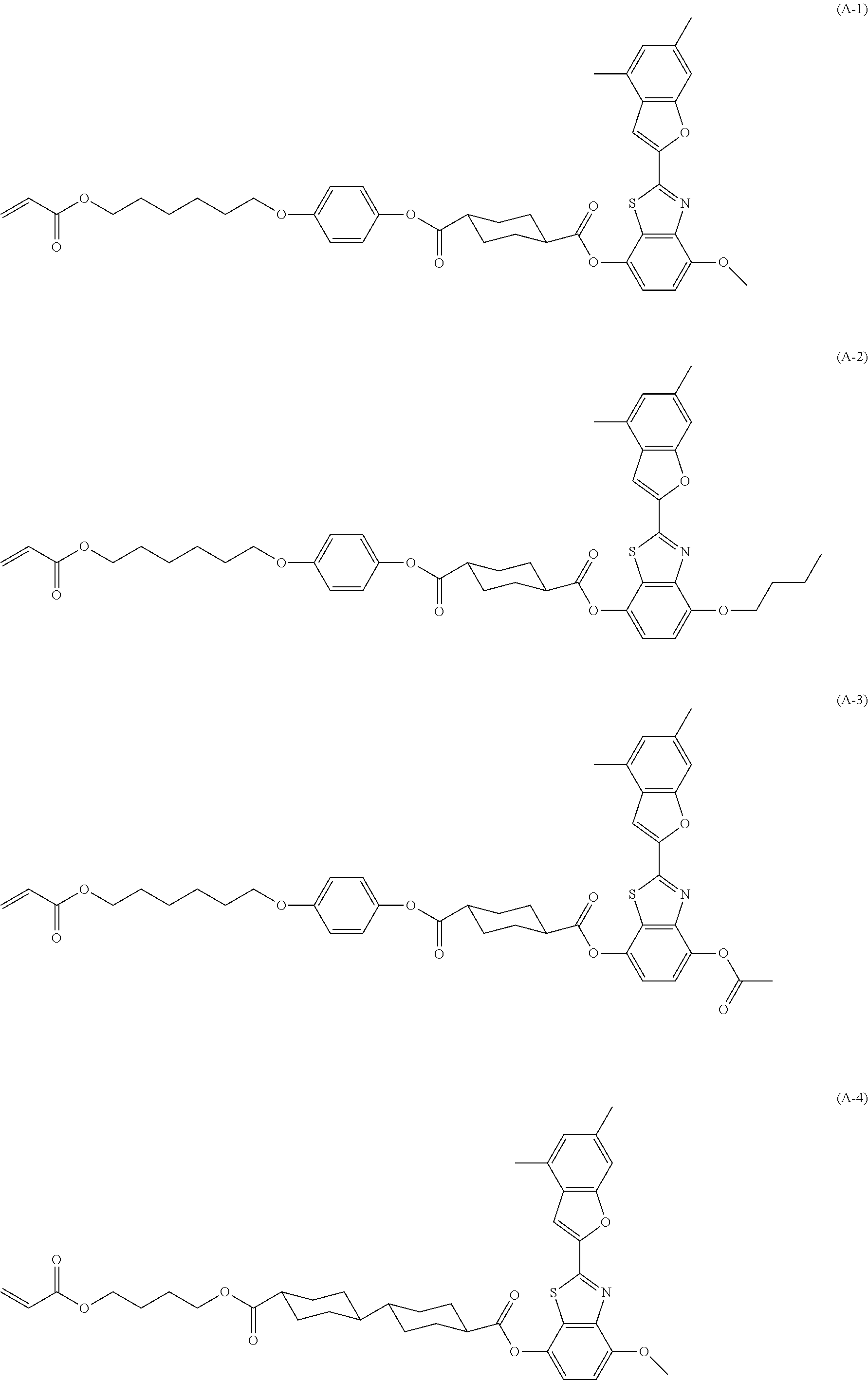

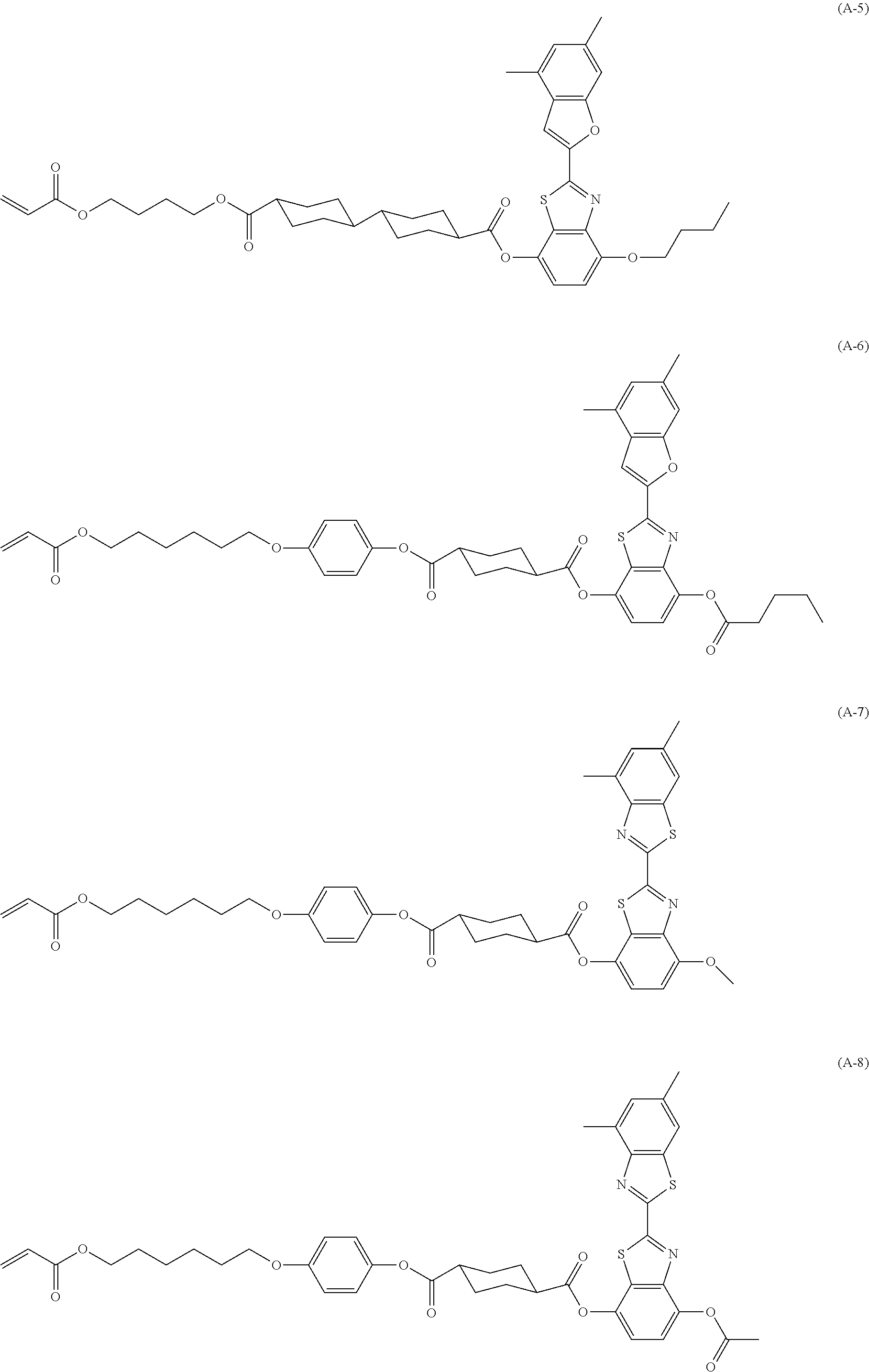

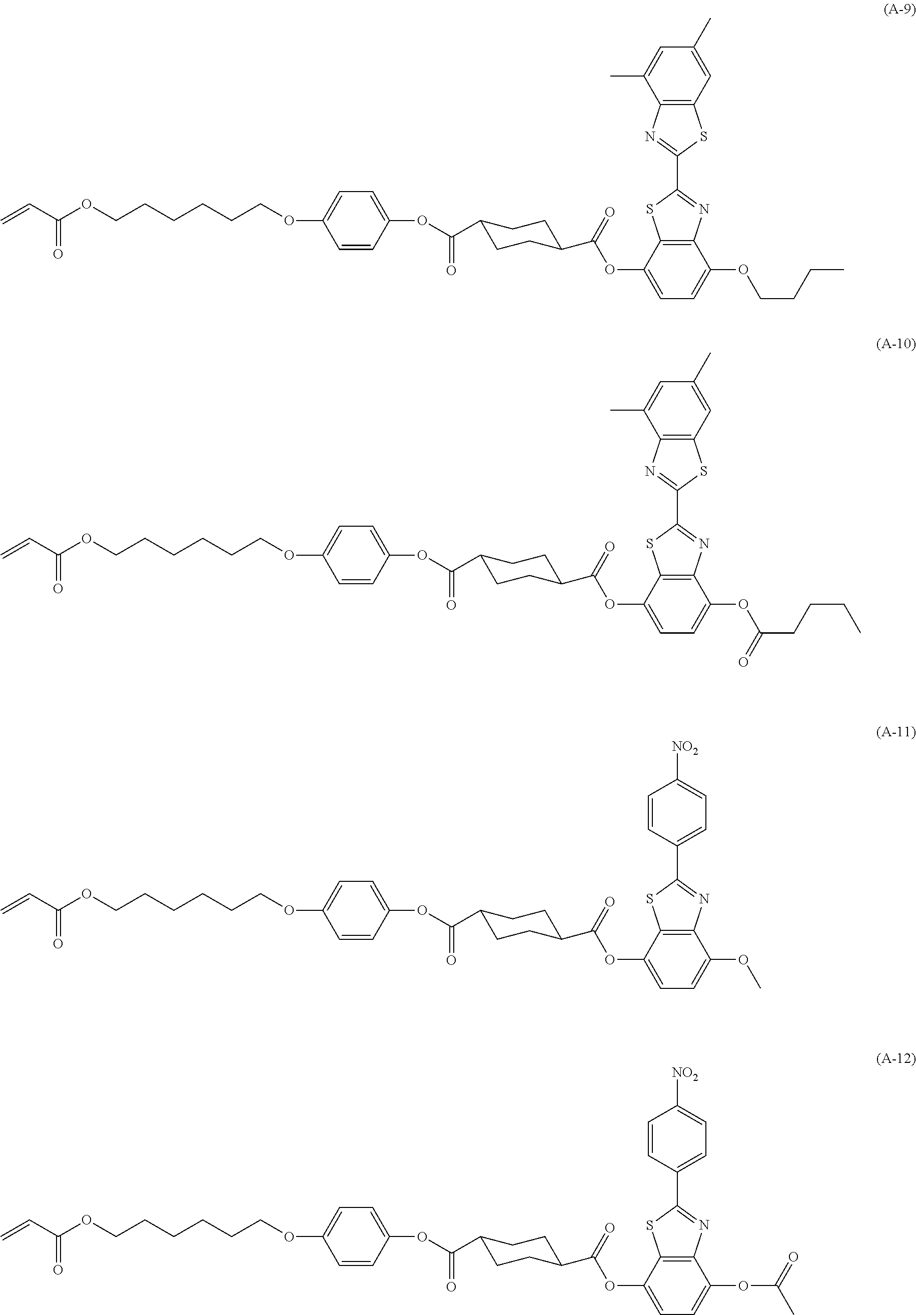

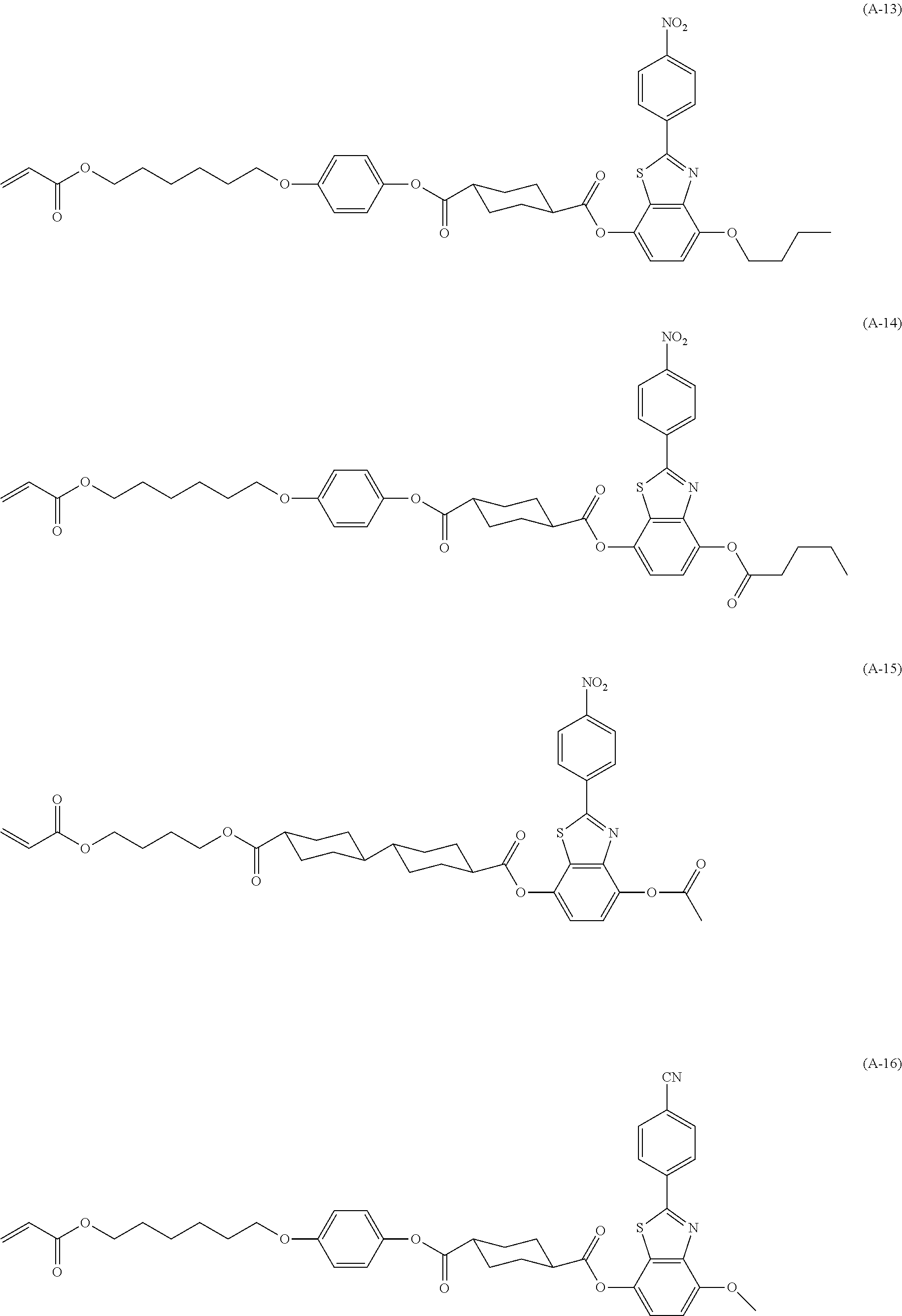

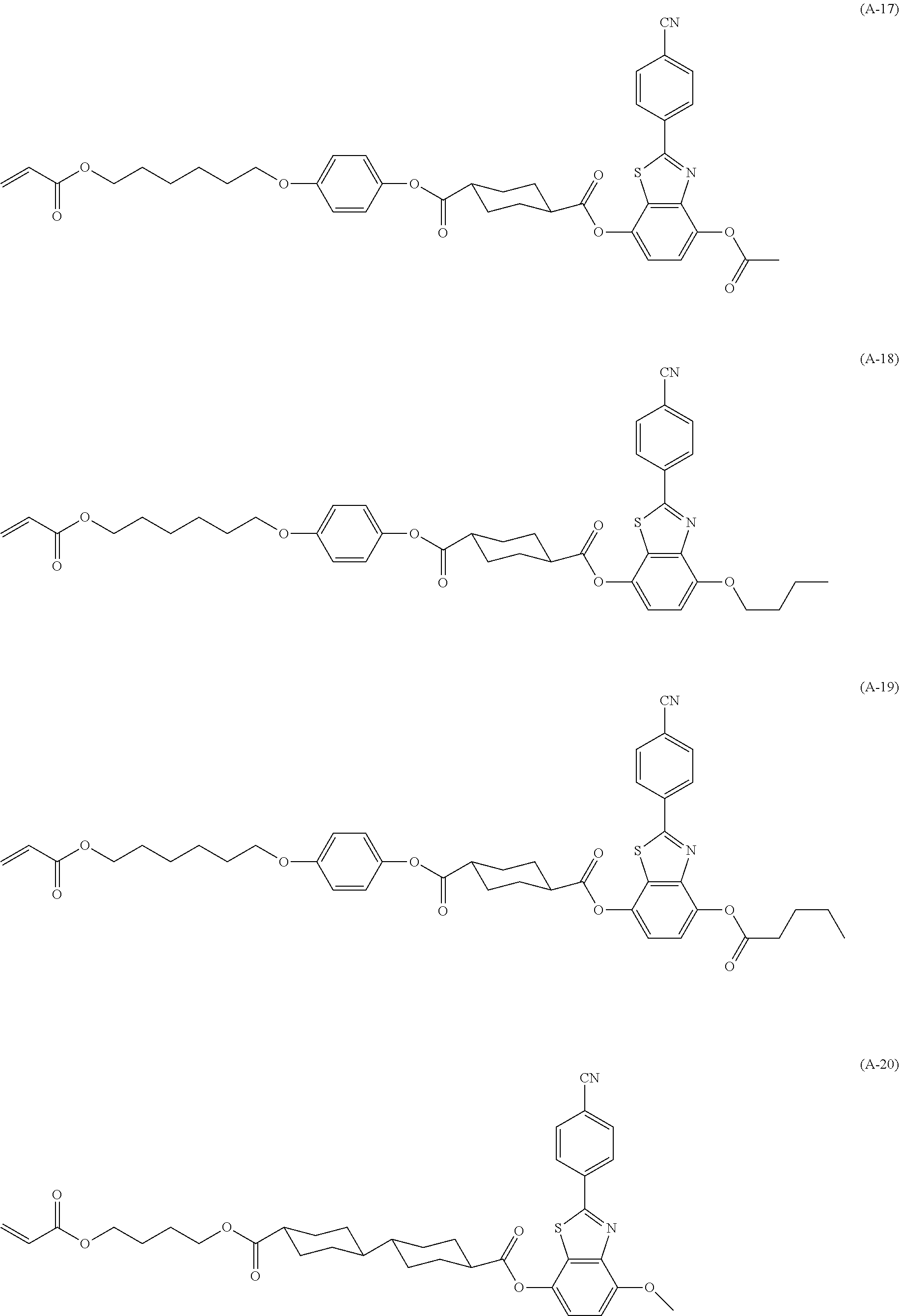

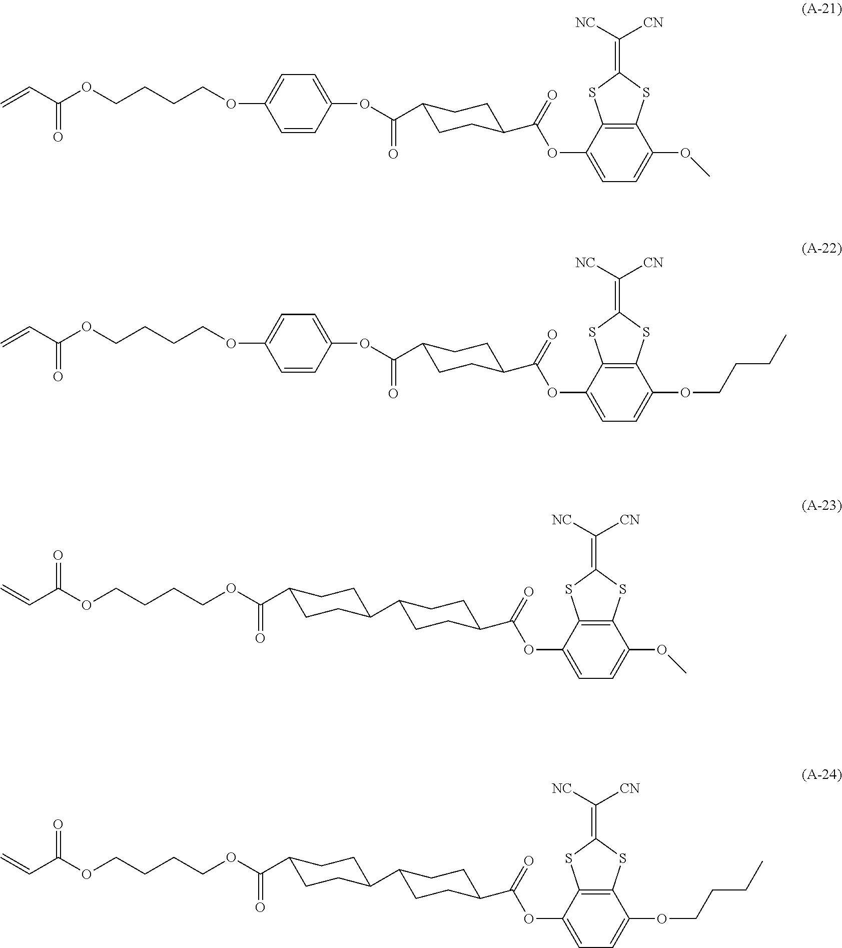

### [Liquid Crystal Compound]

The liquid crystal compound contained in the liquid crystal composition according to the present invention is not particularly limited, and a liquid crystal compound known in the related art can be used.

In general, the liquid crystal compound can be classified into a rod-like type and a disk-like type according to the shape thereof. Each of the types can further be classified into a low-molecular-weight type and a high-molecular-weight type. The “high-molecular-weight” generally refers to a compound having a degree of polymerization of 100 or more (Polymer Physics-Phase Transition Dynamics, written by Masao Doi, p. 2, published by Iwanami Shoten, 1992).

In the present invention, any liquid crystal compound can be used, and it is preferable to use a rod-like liquid crystal compound or a discotic liquid crystal compound (disk-like liquid crystal compound). A mixture of two or more kinds of the rod-like liquid crystal compounds, a mixture of two or more kinds of the disk-like liquid crystal compounds, or a mixture of the rod-like liquid crystal compound and the disk-like liquid crystal compound may be used.

As the rod-like liquid crystal compound, for example, rod-like liquid crystal compounds described in claim 1 of JP1999-513019A (JP-H11-513019A) or paragraphs 0026 to 0098 of JP2005-289980A can be preferably used; and as the discotic liquid crystal compounds, for example, discotic liquid crystal compounds described in paragraphs 0020 to 0067 of JP2007-108732A or paragraphs 0013 to 0108 of JP2010-244038A can be preferably used, but the liquid crystal compounds are not limited thereto.

In the present invention, from the reason of improving durability of the liquid crystal cured layer, it is preferable that the above-described liquid crystal compound has a polymerizable group, and it is more preferable that the liquid crystal compound has two or more polymerizable groups.

Here, suitable examples of the polymerizable group include the polymerizable group represented by any of Formulae (P-1) to (P-20) described above.

In addition, in the present invention, from the reason of further improving the aligning properties, it is preferable that the liquid crystal compound is a compound represented by Formula (B).

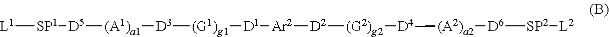

In Formula (B), a1, a2, g1, and g2 each independently represent 0 or 1. Here, at least one of a1 or g1 represents 1 and at least one of a2 or g2 represents 1.

In addition, D¹, D², D³, D⁴, D⁵, and D⁶ each independently represent a single bond, —CO—, —O—, —S—, —C(═S)—, —CR¹R²—, —CR³═CR⁴—, —NR⁵—, or a divalent linking group consisting of a combination of two or more of these groups, where R¹ to R⁵ each independently represent a hydrogen atom, a fluorine atom, or an alkyl group having 1 to 12 carbon atoms.

In addition, G¹ and G² each independently represent an aromatic ring having 6 to 20 carbon atoms, which may have a substituent, or a divalent alicyclic hydrocarbon group having 5 to 20 carbon atoms, which may have a substituent, where one or more of —CH₂—'s constituting the alicyclic hydrocarbon group may be replaced with —O—, —S—, or —NH—.

In addition, A¹ and A² each independently represent an aromatic ring having 6 to 20 carbon atoms, which may have a substituent, or a divalent alicyclic hydrocarbon group having 5 to 20 carbon atoms, which may have a substituent, where one or more of —CH₂—'s constituting the alicyclic hydrocarbon group may be replaced with —O—, —S—, or —NH—.

In addition, SP¹ and SP² each independently represent a single bond or a divalent aliphatic hydrocarbon group having 1 to 20 carbon atoms. Here, one or more of —CH₂—'s constituting the aliphatic hydrocarbon group may be replaced with —O—, —S—, —NH—, —N(Q)-, or —CO—. where Q represents a substituent,

In addition, L′ and L² each independently represent a monovalent organic group, where at least one of L¹ or L² represents a polymerizable group. Here, in a case where Ar² is an aromatic ring represented by Formula (Ar-3), at least one of L¹, L², or L³ or L⁴ in Formula (Ar-3) represents a polymerizable group.

In addition, Ar² represents any aromatic ring selected from the group consisting of groups represented by Formulae (Ar-1) to (Ar-7) described later.

In Formula (B), it is preferable that all of a1, a2, g1, and g2 are 1 for the reason that the liquid crystal composition according to the embodiment of the present invention easily exhibits a liquid crystal state of a smectic phase.

In addition, it is preferable that both of a1 and a2 are 0 and both of g1 and g2 are 1 for the reason that the durability of the liquid crystal cured layer to be produced is improved.

Examples of the divalent linking group represented by one aspect of D¹, D², D³, D⁴, D⁵, and D⁶ in Formula (B) include the same as those described for B¹, E¹, and D¹¹ in Formula (A) described above.

In Formula (B), examples of the aromatic ring having 6 to 20 carbon atoms, represented by one aspect of G¹ and G², include aromatic hydrocarbon rings such as a benzene ring, a naphthalene ring, an anthracene ring, and a phenanthrene ring; and aromatic heterocyclic rings such as a furan ring, a pyrrole ring, a thiophene ring, a pyridine ring, a thiazole ring, and a benzothiazole ring. Among these, a benzene ring (for example, a 1,4-phenyl group) is preferable.

In Formula (B), the divalent alicyclic hydrocarbon group having 5 to 20 carbon atoms, represented by one aspect of G¹ and G², is preferably a 5-membered ring or a 6-membered ring. In addition, the alicyclic hydrocarbon group may be saturated or unsaturated, but a saturated alicyclic hydrocarbon group is preferable. As the divalent alicyclic hydrocarbon group represented by G¹ and G², for example, the description of paragraph [0078] of JP2012-21068A can be referred to, the contents of which are incorporated herein by reference.

In the present invention, G¹ and G² in Formula (B) are each preferably a cycloalkane ring for the reason that the durability of the liquid crystal cured layer to be produced is improved.

Specific examples of the cycloalkane ring include a cyclohexane ring, a cycloheptane ring, a cyclooctane ring, a cyclododecane ring, and a cyclodocosane ring.

Among these, a cyclohexane ring is preferable, a 1,4-cyclohexylene group is more preferable, and a trans-1,4-cyclohexylene group is still more preferable.

In addition, in Formula (B), examples of the substituent which may be included in the aromatic ring having 6 to 20 carbon atoms and the divalent alicyclic hydrocarbon group having 5 to 20 carbon atoms for G¹ and G² include the substituents described in the substituent group A above; and among these, an alkyl group, an alkoxy group, an alkoxycarbonyl group, an alkylcarbonyloxy group, or a halogen atom is preferable.

In Formula (B), examples of the aromatic ring having 6 to 20 carbon atoms, represented by one aspect of A¹ and A², include the same as those described for G¹ and G² in Formula (B) described above.

In addition, in Formula (B), examples of the divalent alicyclic hydrocarbon group having 5 to 20 carbon atoms, represented by one aspect of A¹ and A², include the same as those described for G¹ and G² in Formula (B) described above.

In A¹ and A², examples of the substituent which may be included in the aromatic ring having 6 to 20 carbon atoms or the divalent alicyclic hydrocarbon group having 5 to 20 carbon atoms include the same as those described for the substituent which may be included in each of G¹ and G² in Formula (B) described above.

Examples of the divalent aliphatic hydrocarbon group having 1 to 20 carbon atoms, represented by one aspect of SP¹ and SP² in Formula (B), include the same as those described for F¹ in Formula (A) above.

Examples of the monovalent organic group represented by L¹ and L² in Formula (B) include the same as those described for L³ and L⁴ in Formula (Ar-3) represented by one aspect of Ar¹ in Formula (A) described above.

In addition, suitable examples of the polymerizable group represented by at least one of L¹ or L² include the polymerizable group represented by any of Formulae (P-1) to (P-20) described above.

In Formula (B), as described above, Ar² represents any aromatic ring selected from the group consisting of groups represented by Formulae (Ar-1) to (Ar-7). In Formulae (Ar-1) to (Ar-7), * represents a bonding position to D¹ or D² in Formula (B) described above.

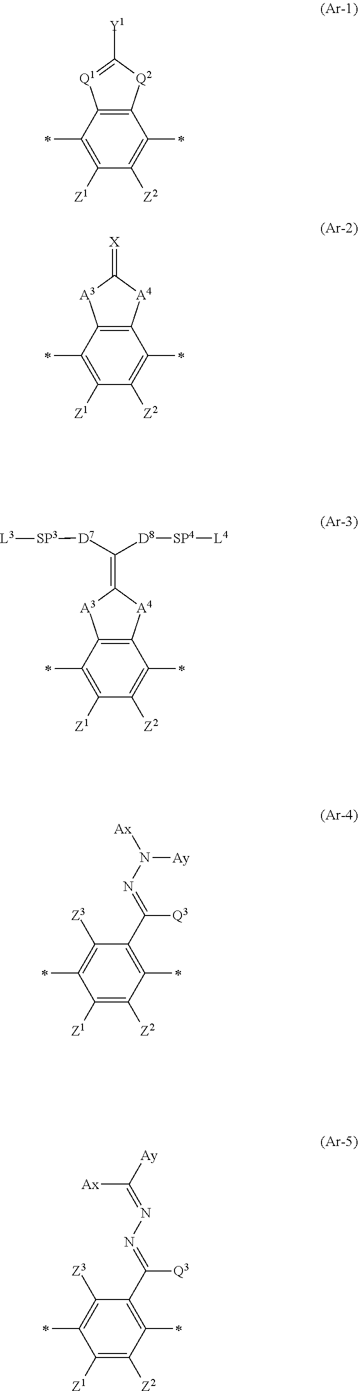

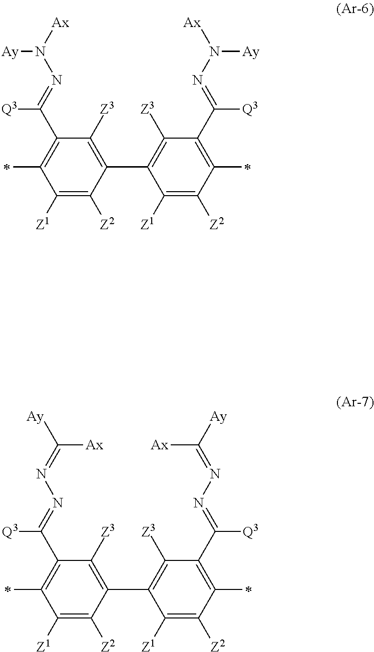

Here, each symbol in Formulae (Ar-1) to (Ar-3) is the same as each symbol in Formulae (Ar-1) to (Ar-3) represented by Ar¹ in Formula (A) described above.

In addition, Z¹, Z², and Z³ in Formulae (Ar-4) to (Ar-7) are the same as Z¹ and Z² in Formulae (Ar-1) to (Ar-3) described above.

In addition, in Formulae (Ar-4) to (Ar-7), Ax represents an organic group having 2 to 30 carbon atoms, which has at least one aromatic ring selected from the group consisting of an aromatic hydrocarbon ring and an aromatic heterocyclic ring.

In addition, in Formulae (Ar-4) to (Ar-7), Ay represents a hydrogen atom, an alkyl group having 1 to 12 carbon atoms, which may have a substituent, or an organic group having 2 to 30 carbon atoms which has at least one aromatic ring selected from the group consisting of an aromatic hydrocarbon ring and an aromatic heterocyclic ring.

Here, the aromatic rings in Ax and Ay may have a substituent, and Ax and Ay may be bonded to each other to form a ring.

In addition, Q³ represents a hydrogen atom or an alkyl group having 1 to 6 carbon atoms, which may have a substituent.

Examples of Ax and Ay include those described in paragraphs to of WO2014/010325A.

In addition, specific examples of the alkyl group having 1 to 6 carbon atoms, represented by Q³, include a methyl group, an ethyl group, a propyl group, an isopropyl group, a n-butyl group, an isobutyl group, a sec-butyl group, a tert-butyl group, a n-pentyl group, and a n-hexyl group; and examples of the substituent include the substituents described in the substituent group A above. Among these, an alkyl group, an alkoxy group, an alkoxycarbonyl group, an alkylcarbonyloxy group, or a halogen atom is preferable.

Examples of the above-described liquid crystal compound include a compound represented by General Formula (1) described in JP2010-084032A (particularly, a compound described in paragraphs [0067] to [0073]), a compound represented by General Formula (II) described in JP2016-053709A (particularly, a compound described in paragraphs [0036] to [0043]), a compound represented by General Formula (1) described in JP2016-081035A (particularly, a compound described in paragraphs [0043] to [0055]), and a compound described in paragraphs to of WO2021/060427A.

Among these, liquid crystal compounds (B-1) to (B-13) represented by the following formulae are preferable as the liquid crystal compound.

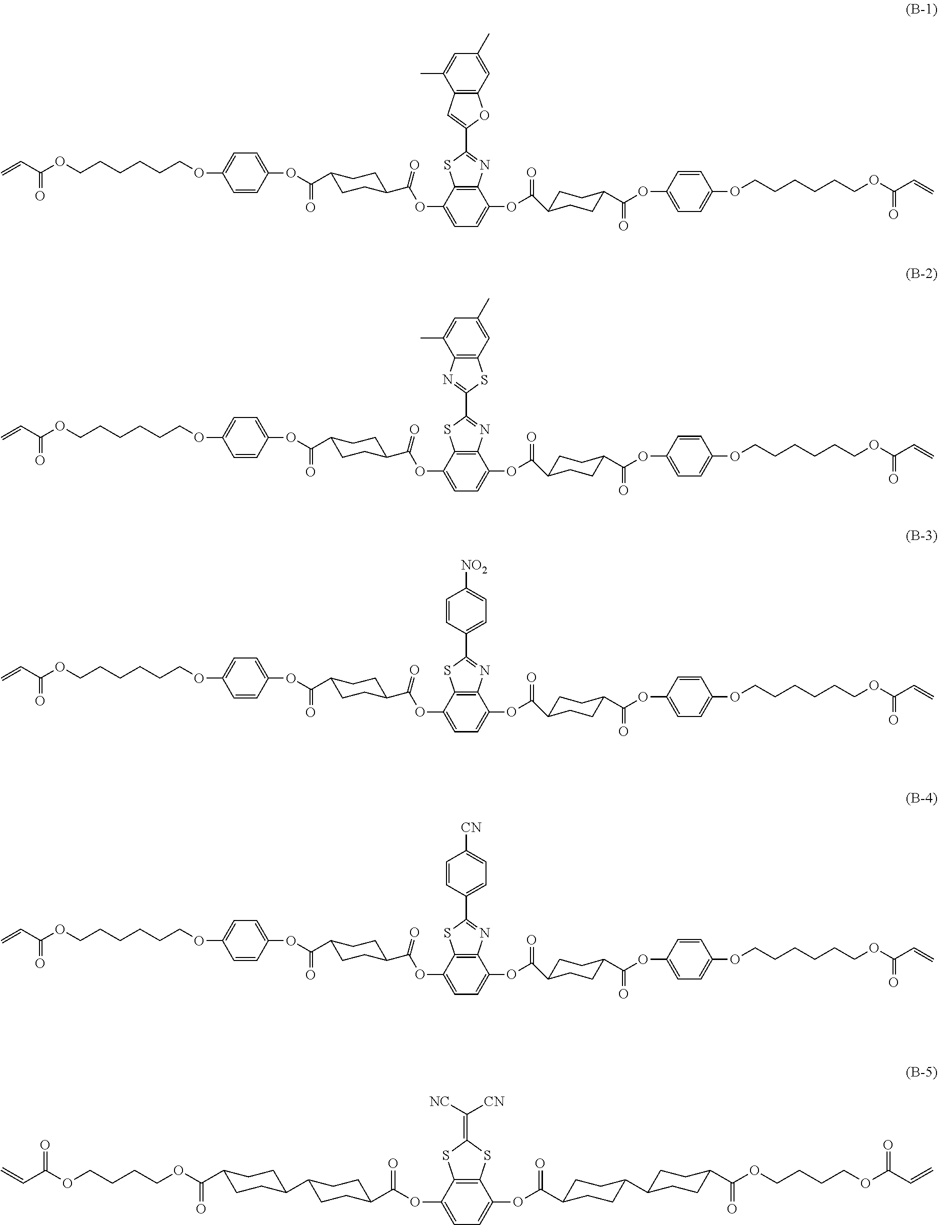

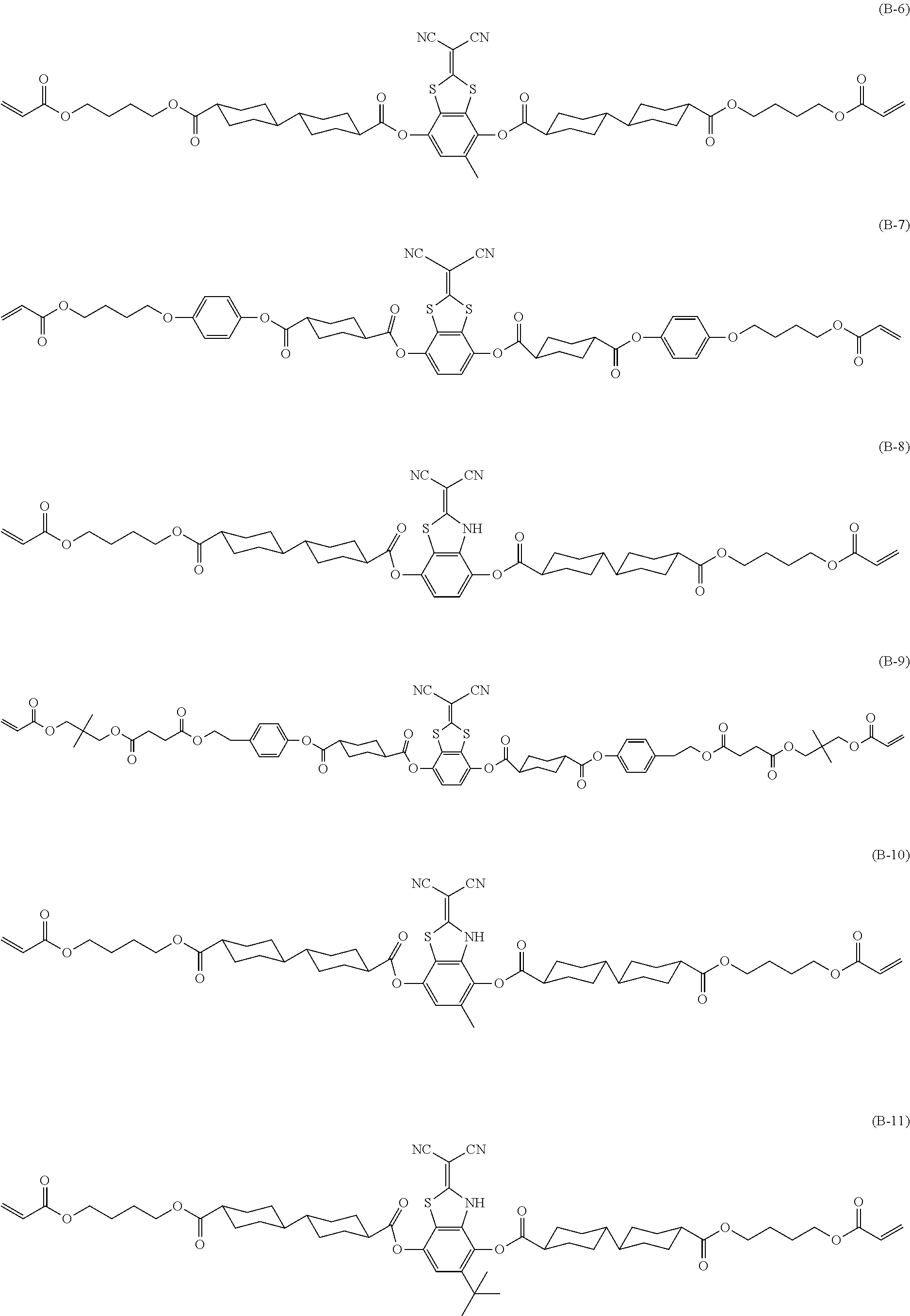

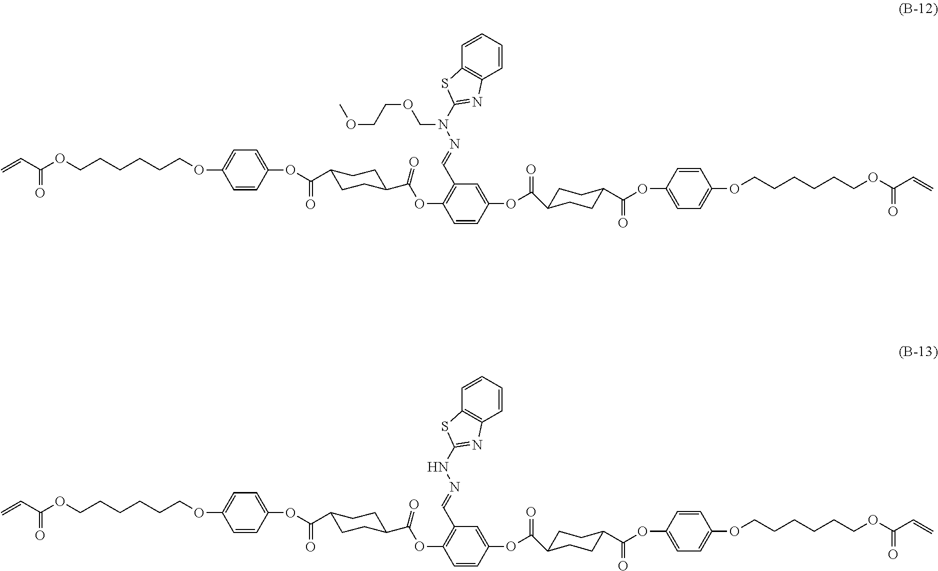

In the present invention, from the reason that the aligning properties are more favorable, a content of the above-described compound represented by Formula (A) is preferably 18% by mass or less, and more preferably 5% to 18% by mass with respect to the total mass of the above-described compound represented by Formula (A) and the above-described liquid crystal compound (particularly, the above-described compound represented by Formula (B)).

In the present invention, from the reason that the aligning properties are more favorable, it is preferable that the above-described compound represented by Formula (A) (specific compound A) and the above-described compound represented by Formula (B) (liquid crystal compound) have the same structure in which Ar¹ in Formula (A) and Ar² in Formula (B) have the same structure.

In the present invention, from the reason that the aligning properties are more favorable, it is preferable that the above-described compound represented by Formula (A) (specific compound A) and the above-described compound represented by Formula (B) (liquid crystal compound) have the same structure in which G¹¹ in Formula (A) and G¹ in Formula (B) have the same structure and A¹¹ in Formula (A) and A¹ in Formula (B) have the same structure.

In addition, in the present invention, from the reason that the aligning properties are more favorable, it is preferable that the above-described compound represented by Formula (A) (specific compound A) and the above-described compound represented by Formula (B) (liquid crystal compound) have the same structure in which D¹¹ in Formula (A) and D¹ in Formula (B) have the same structure and E¹ in Formula (A) and D³ in Formula (B) have the same structure.

### [Other Polymerizable Compounds]

From the viewpoint of alignment temperature and solubility, it is preferable that the liquid crystal composition according to the embodiment of the present invention contains a polymerizable compound having one or more polymerizable groups, in addition to the above-described specific compound A and liquid crystal compound.

Here, the polymerizable group contained in the other polymerizable compounds is not particularly limited; and suitable examples thereof include the polymerizable group represented by any of Formulae (P-1) to (P-20) described above.

As the other polymerizable compounds, from the reason that the durability of the liquid crystal cured layer to be formed is more improved, other polymerizable compounds having two to four polymerizable groups are preferable, and other polymerizable compounds having two polymerizable groups are more preferable.

Examples of the other polymerizable compounds include compounds represented by Formulae (M1), (M2), and (M3) described in paragraphs to of JP2014-077068A, which have liquid crystallinity, and more specifically, specific examples described in paragraphs to of the same publication.

### [Polymerization Initiator]

The liquid crystal composition according to the embodiment of the present invention preferably contains a polymerization initiator.

The polymerization initiator to be used is preferably a photopolymerization initiator capable of initiating a polymerization reaction by irradiation with ultraviolet rays.

Examples of the photopolymerization initiator include α-carbonyl compounds (described in U.S. Pat. Nos. 2,367,661A and 2,367,670A), acyloin ethers (described in U.S. Pat. No. 2,448,828A), α-hydrocarbon-substituted aromatic acyloin compounds (described in U.S. Pat. No. 2,722,512A), polynuclear quinone compounds (described in U.S. Pat. Nos. 3,046,127A and 2,951,758A), combinations of triarylimidazole dimer and p-aminophenyl ketone (described in U.S. Pat. No. 3,549,367A), acridine and phenazine compounds (described in JP1985-105667A (JP-S60-105667A) and U.S. Pat. No. 4,239,850A), oxadiazole compounds (described in U.S. Pat. No. 4,212,970A), and acyl phosphine oxide compounds (described in JP1988-40799B (JP-S63-40799B), JP1993-29234B (JP-H05-29234B), JP1998-95788A (JP-H10-95788A), and JP1998-29997A (JP-H10-29997A)).

In addition, in the present invention, it is also preferable that the polymerization initiator is an oxime-type polymerization initiator; and specific examples of the polymerization initiator include initiators described in paragraphs [0049] to [0052] of WO2017/170443A.

### [Solvent]

From the viewpoint of workability or the like to form the liquid crystal cured layer, the liquid crystal composition according to the embodiment of the present invention preferably contains a solvent.

Specific examples of the solvent include ketones (for example, acetone, 2-butanone, methyl ethyl ketone, methyl isobutyl ketone, cyclohexanone, cyclopentanone, and the like), ethers (for example, dioxane, tetrahydrofuran, and the like), aliphatic hydrocarbons (for example, hexane and the like), alicyclic hydrocarbons (for example, cyclohexane and the like), aromatic hydrocarbons (for example, toluene, xylene, trimethylbenzene, and the like), halogenated carbons (for example, dichloromethane, dichloroethane, dichlorobenzene, chlorotoluene, and the like), esters (for example, methyl acetate, ethyl acetate, butyl acetate, and the like), water, alcohols (for example, ethanol, isopropanol, butanol, cyclohexanol, and the like), cellosolves (for example, methyl cellosolve, ethyl cellosolve, and the like), cellosolve acetates, sulfoxides (for example, dimethyl sulfoxide and the like), and amides (for example, dimethyl formamide, dimethyl acetamide, and the like), and these solvents may be used alone or in a combination of two or more kinds thereof.

### [Leveling Agent]

From the viewpoint of easily controlling alignment, the liquid crystal composition according to the embodiment of the present invention preferably contains a leveling agent.

The leveling agent is preferably a fluorine-based leveling agent or a silicon-based leveling agent for a reason that it has a high leveling effect with respect to the addition amount, and the leveling agent is more preferably a fluorine-based leveling agent from the viewpoint that it is less likely to cause bleeding (bloom or bleed).

Specific examples of the leveling agent include compounds described in paragraphs to [0079] of [0102] JP2007-069471A, a compound represented by General Formula (I) described in JP2013-047204A (in particular, compounds described in paragraphs [0020] to [0032]), a compound represented by General Formula (I) described in JP2012-211306A (in particular, compounds described in paragraphs [0022] to [0029]), a liquid crystal alignment accelerator represented by General Formula (I) described in JP2002-129162A (in particular, compounds described in paragraphs to and [0092] to [0084]), and compounds represented by General Formulae (I), (II), and (III) described in JP2005-099248A (in particular, compounds described in paragraphs [0092] to [0096]). The leveling agent may also function as an alignment control agent described later.

### [Alignment Control Agent]

The liquid crystal composition according to the embodiment of the present invention can contain an alignment control agent as necessary.

With the alignment control agent, in addition to the homogeneous alignment, various alignment states such as homeotropic alignment (vertical alignment), tilt alignment, hybrid alignment, and cholesteric alignment can be formed, and specific alignment states can be controlled and achieved more uniformly and more accurately.

As an alignment control agent which accelerates the homogeneous alignment, for example, a low-molecular-weight alignment control agent or a high-molecular-weight alignment control agent can be used.

With regard to the low-molecular-weight alignment control agent, reference can be made to the description in, for example, paragraphs to of JP2002-20363A, paragraphs [0111] to [0120] of JP2006-106662A, and paragraphs [0021] to [0029] of JP2012-211306A, the contents of which are incorporated herein by reference.

In addition, with regard to the high-molecular-weight alignment control agent, reference can be made to the description in, for example, paragraphs [0021] to [0057] of JP2004-198511A and paragraphs [0121] to [0167] of JP2006-106662A, the contents of which are incorporated herein by reference.

In addition, examples of the alignment control agent which forms or accelerates the homeotropic alignment include a boronic acid compound and an onium salt compound, and specifically, reference can be made to compounds described in paragraphs [0023] to [0032] of JP2008-225281A, paragraphs [0052] to [0058] of JP2012-208397A, paragraphs to of JP2008-026730A, paragraphs [0043] to [0055] of JP2016-193869A, and the like, the contents of which are incorporated herein by reference.

On the other hand, the cholesteric alignment can be achieved by adding a chiral agent to the liquid crystal composition according to the embodiment of the present invention, and it is possible to control the direction of revolution of the cholesteric alignment by its chiral direction.

Incidentally, it is possible to control a pitch of the cholesteric alignment in accordance with an alignment regulating force of the chiral agent.

In a case where an alignment control agent is contained, a content thereof is preferably 0.01% to 10% by mass, and more preferably 0.05% to 5% by mass with respect to the total solid content mass of the composition. In a case where the content is within the range, it is possible to obtain a cured substance which has no precipitation or phase separation, alignment defects, or the like, and is uniform and highly transparent while achieving a desired alignment state.

### [Other Components]

The liquid crystal composition according to the embodiment of the present invention may contain components other than the above-described components; and examples thereof include a surfactant, a tilt angle control agent, an alignment assistant, a plasticizer, and a crosslinking agent.

### [Monofunctional Monomer]

The monofunctional monomer according to the embodiment of the present invention is the above-described compound (specific compound A) represented by Formula (A).

### [Liquid Crystal Cured Layer]

The liquid crystal cured layer according to the embodiment of the present invention is a liquid crystal cured layer obtained by fixing an alignment state of the above-described liquid crystal composition according to the embodiment of the present invention.

Examples of a method for forming the liquid crystal cured layer include a method of using the above-described liquid crystal composition according to the embodiment of the present invention to obtain a desired alignment state, and then fixing an alignment state by polymerization.

Here, polymerization conditions are not particularly limited, but ultraviolet rays are preferably used in the polymerization by light irradiation. An irradiation amount is preferably 10 mJ/cm² to 50 J/cm², more preferably 20 mJ/cm² to 5 J/cm², still more preferably 30 mJ/cm² to 3 J/cm², and particularly preferably 50 mJ/cm² to 1,000 mJ/cm². In order to promote the polymerization reaction, the treatment may be performed under heating conditions.

The liquid crystal cured layer can be formed on any support or alignment film in the optical film described later, or a polarizer in the polarizing plate described later.

The alignment state of the liquid crystal compound in the liquid crystal cured layer according to the embodiment of the present invention may be any of horizontal alignment, vertical alignment, tilt alignment, and twist alignment; and it is preferable that the liquid crystal compound is immobilized in a state of being horizontally aligned with respect to the main surface of the liquid crystal cured layer.

In the present specification, the “horizontal alignment” means that a main surface of the liquid crystal cured layer (or in a case where the liquid crystal cured layer is formed on a member such as a support and an alignment film, a surface of the member) and a major axis direction of the liquid crystal compound are parallel to each other. It is not required for both the main surface of the liquid crystal cured layer and the major axis direction of the liquid crystal compound to be strictly parallel; and in the present specification, the expression means that both the main surface of the liquid crystal cured layer and the major axis direction of the liquid crystal compound are aligned at an angle formed by the major axis direction of the liquid crystal compound and the main surface of the liquid crystal cured layer of less than 10°.

In the liquid crystal cured layer, the angle formed by the major axis direction of the liquid crystal compound and the main surface of the liquid crystal cured layer is preferably 0 to 5°, more preferably 0 to 3°, and still more preferably 0 to 2°.

The liquid crystal cured layer according to the embodiment of the present invention is preferably an optically anisotropic layer, more preferably a positive A-plate or a positive C-plate, and still more preferably a positive A-plate.

Here, the positive A-plate and the positive C-plate are defined as follows.

The positive A-plate satisfies a relationship of Expression (A¹) and the positive C-plate satisfies a relationship of Expression (C1), assuming that a refractive index in a film in-plane slow axis direction (in a direction in which an in-plane refractive index is maximum) is defined as nx, a refractive index in an in-plane direction orthogonal to the in-plane slow axis is defined as ny, and a refractive index in a thickness direction is defined as nz. The positive A-plate has an Rth showing a positive value and the positive C-plate has an Rth showing a negative value.

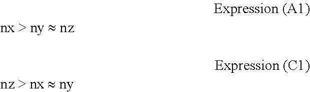

The symbol “≈” encompasses not only a case where both sides are completely the same as each other but also a case where the both sides are substantially the same as each other.

With regard to the positive A-plate, the expression “substantially the same” means that, for example, a case where (ny−nz)× d (in which d is a thickness of a film) is −10 to 10 nm and preferably −5 to 5 nm is also included in “ny≈nz”; and a case where (nx−nz)×d is −10 to 10 nm and preferably −5 to 5 nm is also included in “nx≈nz”. In addition, in the positive C-plate, for example, a case where (nx−ny)×d (in which d is a thickness of a film) is 0 to 10 nm, and preferably 0 to 5 nm is also included in “nx≈ny”.

In a case where the liquid crystal cured layer according to the embodiment of the present invention is the positive A-plate, from the viewpoint that the retardation layer functions as a λ/4 plate, Re(550) is preferably 100 to 180 nm, more preferably 120 to 160 nm, still more preferably 130 to 150 nm, and particularly preferably 130 to 145 nm.

Here, the “λ/4 plate” is a plate having a λ/4 function, specifically, a plate having a function of converting linearly polarized light having a specific wavelength into circularly polarized light (or converting circularly polarized light into linearly polarized light).

In a case where the liquid crystal composition according to the embodiment of the present invention contains a dichroic substance, the liquid crystal cured layer according to the embodiment of the present invention can be used as a polarizer (light absorption anisotropic film).

### [Optical Film]

The optical film according to the embodiment of the present invention is an optical film including the liquid crystal cured layer according to the embodiment of the present invention.

A structure of the optical film will be described with reference to the FIGURE. The FIGURE is a schematic cross-sectional view showing an example of the optical film.

The FIGURE is a schematic view, and the thicknesses relationship, the positional relationship, and the like of the respective layers are not necessarily consistent with actual ones; and a support and an alignment film shown in the FIGURE are optional constitutional members.

An optical film 10 shown in the FIGURE includes, in the following order, a support 16, an alignment film 14, and a liquid crystal cured layer 12 as a cured substance of the liquid crystal composition according to the embodiment of the present invention.

In addition, the liquid crystal cured layer 12 may be a laminate of two or more different liquid crystal cured layers. For example, in a case where the polarizing plate according to the embodiment of the present invention, which will described later, is used as a circularly polarizing plate or the optical film according to the embodiment of the present invention is used as an optical compensation film of an in-plane-switching (IPS) mode or fringe-field-switching (FFS) mode liquid crystal display device, it is preferable that the liquid crystal cured layer 12 is a laminate of a positive A-plate and a positive C-plate.

In addition, the liquid crystal cured layer may be peeled off from the support and used alone as the optical film.

Hereinafter, various members used for the optical film will be described in detail.

### [Liquid Crystal Cured Layer]

The liquid crystal cured layer included in the optical film according to the embodiment of the present invention is the above-described liquid crystal cured layer according to the embodiment of the present invention.

In the optical film, a thickness of the above-described liquid crystal cured layer is not particularly limited, but is preferably 0.1 to 10 μm and more preferably 0.5 to 5 μm.

### [Support]

The optical film may include a support as a base material for forming the liquid crystal cured layer as described above.

Such a support is preferably transparent. Specifically, a light transmittance thereof is preferably 80% or more.

Examples of such a support include a glass substrate and a polymer film. Examples of a material of the polymer film include cellulose-based polymers; acrylic polymers having an acrylic acid ester polymer such as polymethyl methacrylate and a lactone ring-containing polymer; thermoplastic norbornene-based polymers; polycarbonate-based polymers; polyester-based polymers such as polyethylene terephthalate and polyethylene naphthalate; styrene-based polymers such as polystyrene and an acrylonitrile-styrene copolymer (AS resin); polyolefin-based polymers such as polyethylene, polypropylene, and an ethylene-propylene copolymer; vinyl chloride-based polymers; amide-based polymers such as nylon and aromatic polyamide; imide-based polymers; sulfone-based polymers; polyether sulfone-based polymers; polyether ether ketone-based polymers; polyphenylene sulfide-based polymers; vinylidene chloride-based polymers; vinyl alcohol-based polymers; vinyl butyral-based polymers; arylate-based polymers; polyoxymethylene-based polymers; epoxy-based polymers; and polymers obtained by mixing these polymers.

In addition, an aspect in which a polarizer described later may also function as the support is also adopted.

A thickness of the above-described support is not particularly limited, but is preferably 5 to 60 μm, and more preferably 5 to 40 μm.

### [Alignment Film]

In the optical film, the liquid crystal cured layer is preferably formed on a surface of an alignment film (particularly, a photo-alignment film described later). In a case where the optical film includes any of the above-described supports, the alignment film may be interposed between the support and the liquid crystal cured layer. In addition, an aspect in which the above-described support may also function as the alignment film is also adopted.

The alignment film may be any film as long as it has a function of horizontally aligning the polymerizable liquid crystal compound contained in the composition.

The alignment film generally contains a polymer as a main component. A polymer material for the alignment film is described in many documents, and many commercially available products can be used.

As the polymer material for the alignment film, a polyvinyl alcohol, a polyimide, or a derivative thereof is preferable, and a modified or unmodified polyvinyl alcohol is more preferable.

Examples of the alignment film which may be included in the optical film include alignment films described in Line 24 on Page 43 to Line 8 on Page 49 of WO01/88574A; alignment films consisting of modified polyvinyl alcohols described in paragraphs [0071] to [0095] of JP3907735B; and liquid crystal alignment films formed using a liquid crystal aligning agent described in JP2012-155308A.

Since an object does not come into contact with a surface of the alignment film in the formation of the alignment film and the deterioration of a surface condition can be prevented, it is preferable to use a photo-alignment film as the alignment film.

The photo-alignment film is not particularly limited; but an alignment film formed by a polymer material such as a polyamide compound and a polyimide compound described in paragraphs [0024] to [0043] of WO2005/096041A, a liquid crystal alignment film formed by a liquid crystal aligning agent having a photo-aligned group described in JP2012-155308A, trade name LPP-JP265CP manufactured by Rolic Technologies Ltd., or the like can be used.

A thickness of the alignment film is not particularly limited, but from the viewpoint of forming a liquid crystal cured layer having a uniform film thickness by relaxing the surface roughness which can be present on the support, the thickness is preferably 0.01 to 10 μm, more preferably 0.01 to 1 μm, and still more preferably 0.01 to 0.5 μm.

### [Other Liquid Crystal Cured Layers]

In the optical film, it is preferable that the liquid crystal cured layer is formed on a surface of other liquid crystal cured layers.

Here, examples of the other liquid crystal cured layers include a liquid crystal cured layer obtained by fixing an alignment state of a composition obtained by removing the specific compound A from the above-described liquid crystal composition according to the embodiment of the present invention. Specific examples thereof include a liquid crystal cured layer obtained by fixing an alignment state of a composition containing the above-described liquid crystal compound, a polymerization initiator, a leveling agent, a solvent, and the like.

### [Ultraviolet Absorber]

It is preferable that the optical film contains an ultraviolet (UV) absorber, taking an effect of external light (particularly, ultraviolet rays) into consideration.

The ultraviolet absorber may be contained in the liquid crystal cured layer, or may be contained in a member other than the liquid crystal cured layer, constituting the optical film. Suitable examples of the member other than the liquid crystal cured layer include the support.

As the ultraviolet absorber, any one of ultraviolet absorbers known in the related art, which can express ultraviolet absorptivity, can be used. Among such ultraviolet absorbers, a benzotriazole-based ultraviolet absorber or a hydroxyphenyltriazine-based ultraviolet absorber is preferable from the viewpoint that it has high ultraviolet absorptivity and ultraviolet absorbing ability (ultraviolet-shielding ability) used for an image display device is obtained.

In addition, in order to broaden ultraviolet absorbing ranges, two or more kinds of ultraviolet absorbers having different maximum absorption wavelengths are also preferably used in combination.

Examples of the ultraviolet absorber include compounds described in paragraphs [0258] and [0259] of JP2012-18395A and compounds described in paragraphs to of JP2007-72163A.

In addition, as a commercially available product thereof, for example, Tinuvin 400, Tinuvin 405, Tinuvin 460, Tinuvin 477, Tinuvin 479, Tinuvin 1577, or the like (all manufactured by BASF) can be used.

### [Polarizing Plate]

The polarizing plate according to the embodiment of the present invention includes the above-described optical film according to the embodiment of the present invention and a polarizer.

In addition, in a case where the above-described liquid crystal cured layer according to the embodiment of the present invention is a λ/4 plate (positive A-plate), the polarizing plate according to the embodiment of the present invention can be used as a circularly polarizing plate.

In addition, in the polarizing plate according to the embodiment of the present invention, in a case where the above-described liquid crystal cured layer according to the embodiment of the present invention is a λ/4 plate (positive A-plate), an angle between a slow axis of the λ/4 plate and an absorption axis of the polarizer, which will be described later, is preferably 30° to 60°, more preferably 40° to 50°, still more preferably 42° to 48°, and particularly preferably 45°.

Here, the “slow axis” of the λ/4 plate means a direction in which a refractive index in the plane of the λ/4 plate is maximum, and the “absorption axis” of the polarizer means a direction in which an absorbance is highest.

In addition, the polarizing plate according to the embodiment of the present invention can also be used as an optical compensation film for the IPS mode or FFS mode liquid crystal display device.

In a case where the polarizing plate of the present invention is used as an optical compensation film for the IPS mode or FFS mode liquid crystal display device, the above-described liquid crystal cured layer according to the embodiment of the present invention can be used as at least one plate of a laminate of a positive A-plate and a positive C-plate, and it is preferably a positive A-plate. In this case, it is preferable that the angle between the slow axis of the positive A-plate and the absorption axis of the polarizer described later is orthogonal or parallel, and specifically, the angle between the slow axis of the positive A-plate and the absorption axis of the polarizer described later is more preferably 0° to 5° or 85° to 95°.

In addition, in a case where the polarizing plate according to the embodiment of the present invention has a laminate of the polarizer, the positive C-plate, and the positive A-plate in this order, the angle between the slow axis of the positive A-plate and the absorption axis of the polarizer is still more preferably parallel to each other.

Similarly, in a case where the polarizing plate according to the embodiment of the present invention has a laminate of the polarizer, the positive A-plate, and the positive C-plate in this order, the angle between the slow axis of the positive A-plate and the absorption axis of the polarizer is still more preferably orthogonal to each other.

In a case where the polarizing plate according to the embodiment of the present invention is used in a liquid crystal display device described later, it is preferable that the angle formed by the slow axis of the liquid crystal cured layer and the absorption axis of the polarizer described later is parallel or orthogonal to each other.

In the present specification, the term “parallel” does not require that both the angle formed by the slow axis of the liquid crystal cured layer and the absorption axis of the polarizer are strictly parallel, but means that an angle between one and the other is less than 10°. In addition, in the present specification, the term “orthogonal” does not require that both the angle formed by the slow axis of the liquid crystal cured layer and the absorption axis of the polarizer are strictly orthogonal, but means that an angle between one and the other is more than 80° and less than 100°.

### [Polarizer]

The polarizer of the polarizing plate according to the embodiment of the present invention is not particularly limited as long as the polarizer is a member having a function of converting light into specific linearly polarized light, and a known absorptive type polarizer and reflective type polarizer in the related art can be used.

An iodine-based polarizer, a dye-based polarizer using a dichroic dye, a polyene-based polarizer, or the like is used as the absorptive type polarizer. The iodine-based polarizer and the dye-based polarizer include a coating type polarizer and a stretching type polarizer, and any one of these polarizers can be applied. However, a polarizer which is produced by allowing polyvinyl alcohol to adsorb iodine or a dichroic dye and performing stretching is preferable.

In addition, examples of a method of obtaining a polarizer by performing stretching and dyeing in a state of a laminated film in which a polyvinyl alcohol layer is formed on a substrate include methods disclosed in JP5048120B, JP5143918B, JP4691205B, JP4751481B, and JP4751486B, and known technologies related to these polarizers can be preferably used.

A polarizer in which thin films having different birefringence are laminated, a wire grid type polarizer, a polarizer in which a cholesteric liquid crystal having a selective reflection range and a ¼ wavelength plate are combined, or the like is used as the reflective type polarizer.

Among these, from the viewpoint of more excellent adhesiveness, a polarizer containing a polyvinyl alcohol-based resin (polymer including-CH₂—CHOH— as a repeating unit; in particular, at least one selected from the group consisting of polyvinyl alcohol and an ethylene-vinyl alcohol copolymer) is preferable.

In the present invention, a thickness of the polarizer is not particularly limited, but is preferably 3 μm to 60 μm, more preferably 5 μm to 30 μm, and still more preferably 5 μm to 15 μm.

### [Pressure Sensitive Adhesive Layer]

The polarizing plate according to the embodiment of the present invention may include a pressure sensitive adhesive layer arranged between the liquid crystal cured layer in the optical film according to the embodiment of the present invention and the polarizer.

The pressure sensitive adhesive layer used for lamination of the liquid crystal cured layer and the polarizer is, for example, a substance in which a ratio (tan δ=G″/G′) of loss elastic modulus G″ to storage elastic modulus G′ is 0.001 to 1.5, where G′ and G″ are measured with a dynamic viscoelasticity measurement. Such a substance includes a so-called pressure sensitive adhesive or easily creepable substance. Examples of the pressure sensitive adhesive which can be used in the present invention include a polyvinyl alcohol-based pressure sensitive adhesive, but the pressure sensitive adhesive is not limited thereto.

### [Image Display Device]

The image display device according to the embodiment of the present invention is an image display device including the optical film according to the embodiment of the present invention or the polarizing plate according to the embodiment of the present invention.

A display element used in the image display device is not particularly limited, and examples thereof include a liquid crystal cell, an organic electroluminescent (hereinafter, simply referred to as “EL”) display panel, and a plasma display panel. Among these, a liquid crystal cell or an organic EL display panel is preferable, and a liquid crystal cell is more preferable.

That is, as the image display device, a liquid crystal display device using a liquid crystal cell as the display element or an organic EL display device using an organic EL display panel as the display element is preferable, and the liquid crystal display device is more preferable.

### [Liquid Crystal Display Device]

A liquid crystal display device as an example of the image display device is a liquid crystal display device including the above-described polarizing plate and a liquid crystal cell.

Among polarizing plates provided on both sides of the liquid crystal cell, it is preferable that the above-described polarizing plate is used as a polarizing plate on the front side, and it is more preferable that the above-described polarizing plate is used as polarizing plates on the front and rear sides.

Hereinafter, the liquid crystal cell constituting the liquid crystal display device will be described in detail.

### <Liquid Crystal Cell>

It is preferable that the liquid crystal cell used in the liquid crystal display device is in a vertical alignment (VA) mode, an optically compensated bend (OCB) mode, an in-plane-switching (IPS) mode, a fringe-field-switching (FFS) mode, or a twisted nematic (TN) mode, but is not limited thereto.

In the liquid crystal cell in a TN mode, rod-like liquid crystalline molecules are substantially horizontally aligned at the time of no voltage application and further twisted aligned at 60° to 120°. The liquid crystal cell in a TN mode is most frequently used as a color TFT liquid crystal display device and is described in a plurality of documents.

In the liquid crystal cell in a VA mode, rod-like liquid crystalline molecules are substantially vertically aligned at the time of no voltage application. The concept of the liquid crystal cell in a VA mode includes (1) a liquid crystal cell in a VA mode in a narrow sense where rod-like liquid crystalline molecules are aligned substantially vertically at the time of no voltage application and substantially horizontally at the time of voltage application (described in JP1990-176625A (JP-H2-176625A)), (2) a liquid crystal cell (in an MVA mode) (SID97, described in Digest of tech. Papers (proceedings) 28 (1997) 845) in which the VA mode is formed to have multi-domain in order to expand the viewing angle, (3) a liquid crystal cell in a mode (n-ASM mode) in which rod-like liquid crystalline molecules are substantially vertically aligned at the time of no voltage application and twistedly multi-domain aligned at the time of voltage application (described in proceedings of Japanese Liquid Crystal Conference, pp. 58 to 59 (1998)), and (4) a liquid crystal cell in a SURVIVAL mode (presented at LCD International 98). In addition, the liquid crystal cell in the VA mode may be any of a patterned vertical alignment (PVA) type, an optical alignment type, or a polymer-sustained alignment (PSA). The details of these modes are described in JP2006-215326A and JP2008-538819A.

In an IPS mode liquid crystal cell, rod-like liquid crystal molecules are substantially aligned parallel to a substrate and application of an electric field parallel to a surface of the substrate causes the liquid crystal molecules to respond planarly. In the IPS mode, black display is carried out in a state where no electric field is applied, and absorption axes of a pair of upper and lower polarizing plates are orthogonal to each other. A method of reducing light leakage during black display in an oblique direction and improve the viewing angle using an optical compensation sheet is disclosed in JP1998-54982A (JP-H10-54982A), JP1999-202323A (JP-H11-202323A), JP1997-292522A (JP-H9-292522A), JP1999-133408A (JP-H11-133408A), JP1999-305217A (JP-H11-305217A), JP1998-307291A (JP-H10-307291A), and the like.

### [Organic EL Display Device]

Examples of the organic EL display device which is an example of the image display device include an aspect which includes, from a viewing side, a polarizer, a λ/4 plate consisting of the above-described liquid crystal cured layer (positive A-plate), and an organic EL display panel in this order.

In addition, the organic EL display panel is a display panel formed of an organic EL element obtained by sandwiching an organic light emitting layer (organic electroluminescence layer) between electrodes (between a cathode and an anode). The configuration of the organic EL display panel is not particularly limited, and a known configuration is employed.

### EXAMPLES

Hereinafter, the present invention will be described in more detail with reference to Examples. The materials, amounts used, proportions, treatment contents, treatment procedures, and the like shown in the following examples can be modified as appropriate in the range of not departing from the spirit of the present invention. Therefore, the scope of the present invention should not be construed as being limited to Examples.

### Synthesis of Specific Compound (A-1)

### Compound (A-1-0)

A compound (A-1-0) was synthesized by the following scheme according to a synthesis method described in JP2012-97078A.

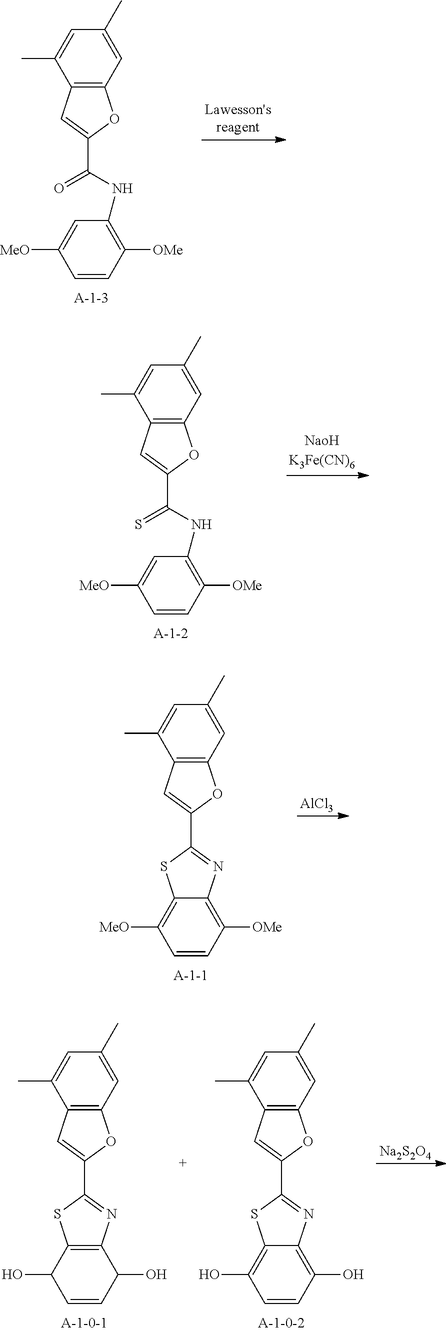

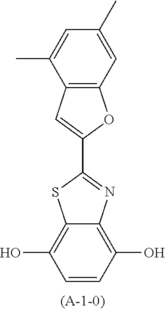 [SMILES: Cc1cc(C)c2cc(-c3nc4c(O)ccc(O)c4s3)oc2c1]

Next, a specific compound (A-1) was synthesized by the following scheme.

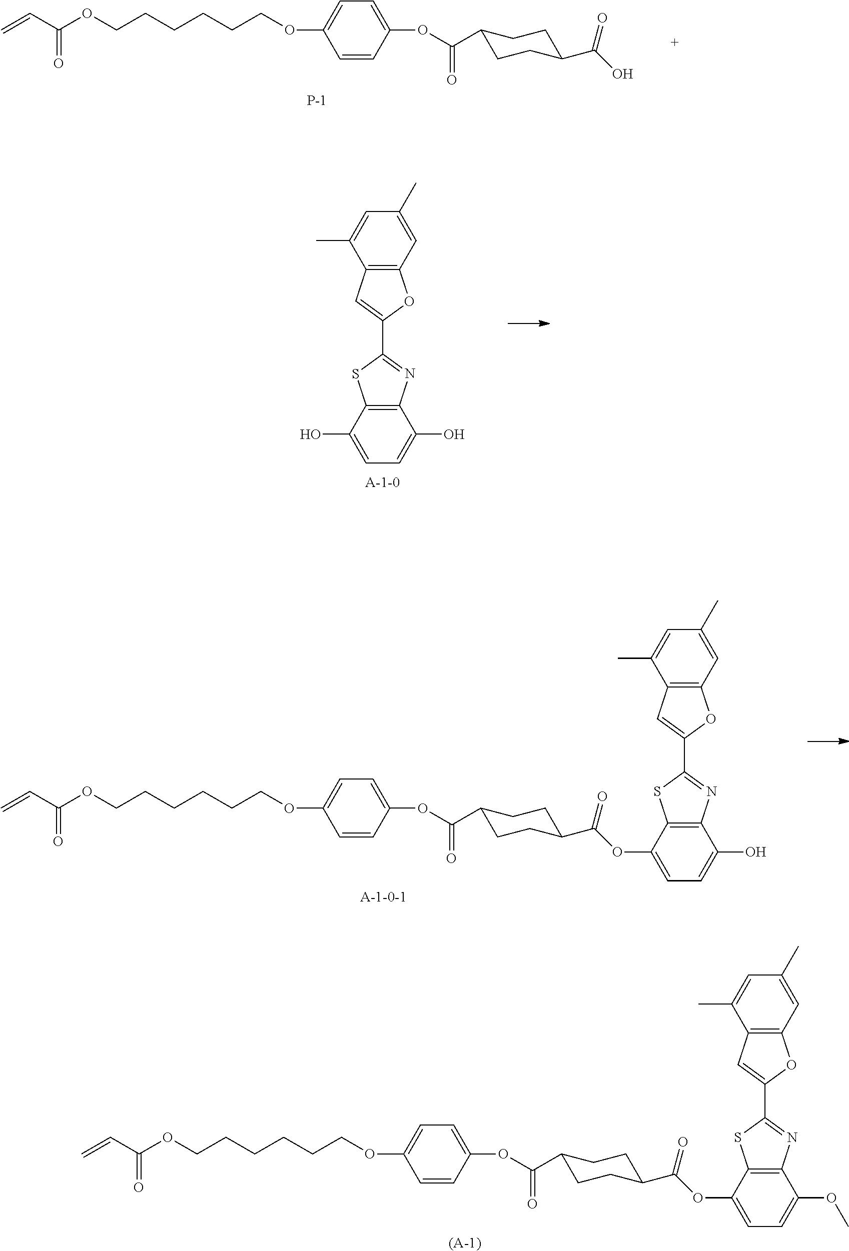

Specifically, in a 1 L three-neck flask, a carboxylic acid (62.5 g, 0.16 mol) of Formula (P-1) synthesized with reference to JP2010-31223A was mixed with A-1-0 (49.8 g, 0.16 mol) previously synthesized and 700 mL of chloroform (manufactured by FUJIFILM Wako Pure Chemical Corporation), 62.5 g of EDCI·HCl (manufactured by Tokyo Chemical Industry Co., Ltd.) was added thereto in small amounts, and the mixture was reacted at room temperature for 5 hours. After the reaction, the reaction mixture was purified by silica gel column chromatography using ethyl acetate and hexane to obtain 5.0 g of A-1-0-1.

Next, 5 g (0.007 mol) of A-1-0-1, 50 mL of DMF (manufactured by FUJIFILM Wako Pure Chemical Corporation), and 6.8 g (0.021 mol) of cesium carbonate (manufactured by FUJIFILM Wako Pure Chemical Corporation) were mixed in a 100 mL three-neck flask and stirred at room temperature, 2 g of methyl iodide (manufactured by FUJIFILM Wako Pure Chemical Corporation) was added dropwise thereto, and the mixture was reacted at room temperature for 24 hours. Thereafter, the reaction mixture was subjected to a reduced pressure filtration, and the filtrate was purified by silica gel column chromatography using ethyl acetate and hexane to obtain 1.5 g of a specific compound (A-1).

The result of mass spectrometry (MS) of the obtained specific compound (A-1) is shown below.

m/z: 725.27 (100.0%), 726.27 (46.0%), 727.27 (12.1%), 727.26 (4.5%), 728.27 (3.0%), 728.28 (1.4%)

### [Other Ether-Based Specific Compounds]

In addition, for a specific compound in which X¹ in Formula (A) is represented by —OR^{L1}, the compound was synthesized by a similar synthesis method to the specific compound (A-1) by changing the raw material.

### Synthesis of Specific Compound (A-3)

A specific compound (A-3) was synthesized by the following scheme.

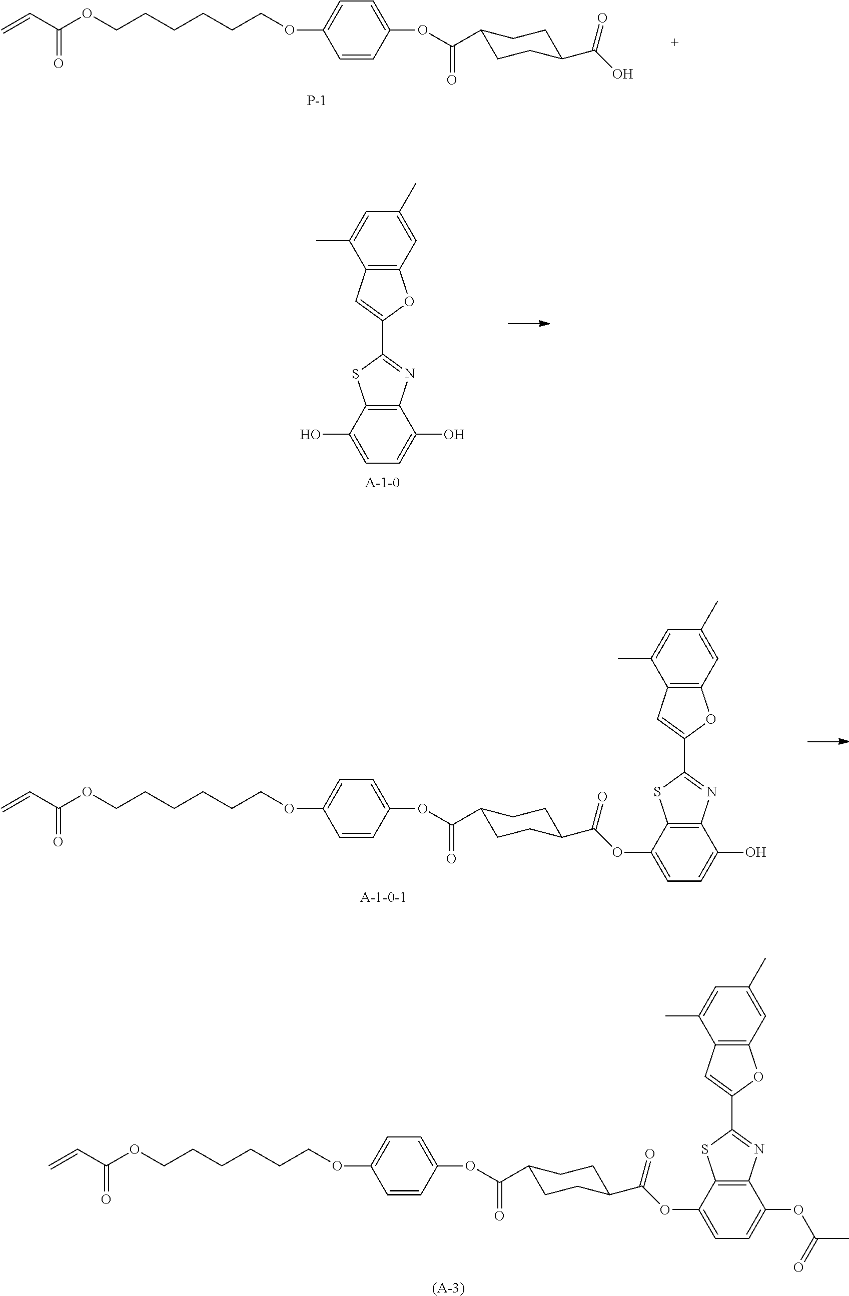

Specifically, 5 g (0.007 mol) of A-1-0-1 synthesized in the middle of the synthesis of the specific compound (A-1), 0.85 g (0.0084 mol) of triethylamine (manufactured by FUJIFILM Wako Pure Chemical Corporation), and 30 mL of tetrahydrofuran (THF) (manufactured by FUJIFILM Wako Pure Chemical Corporation) were mixed in a 200 mL three-neck flask and stirred at room temperature, 0.66 g (0.0084 mol) of acetyl chloride (manufactured by FUJIFILM Wako Pure Chemical Corporation) was added dropwise thereto, and the mixture was reacted at room temperature for 3 hours. Thereafter, the reaction mixture was subjected to a liquid-liquid extraction with ethyl acetate and water, and the obtained organic layer was distilled off under reduced pressure. The obtained crude product was dissolved in 3 g of methylene chloride, and the solution was purified by silica gel column chromatography using ethyl acetate and hexane to obtain 2.5 g of a specific compound (A-3).

The result of mass spectrometry of the obtained specific compound (A-3) is shown below.

m/z: 753.26 (100.0%), 754.26 (47.0%), 755.27 (12.5%), 755.26 (5.1%), 756.27 (2.6%), 756.26 (2.2%)

### [Other Ester-Based Specific Compounds]

In addition, for a specific compound in which X¹ in Formula (A) is represented by —O—(C—O)—R^{L2}, the compound was synthesized by a similar synthesis method to the specific compound (A-3) by changing the raw material.

### Example 1

A liquid crystal composition 1 having the following formulation was prepared.

TABLE
Liquid crystal composition 1

| | |
|---|---|
| Liquid crystal compound (B-1) shown below | 222.5 parts by mass |
| Specific compound (A-1) shown below | 47.0 parts by mass |
| Polymerizable compound (D-1) shown below | 3.0 parts by mass |
| Polymerization initiator S1 shown below | 1.5 parts by mass |
| Leveling agent P1 shown below | 0.1 parts by mass |
| Cyclopentanone | 92.5 parts by mass |
| Methyl ethyl ketone | 64.2 parts by mass |

Liquid crystal compound (B-1) 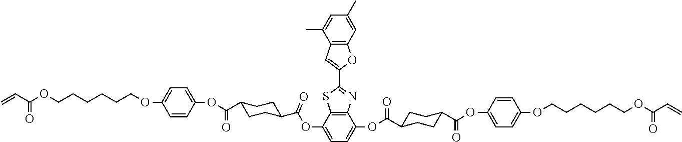 [SMILES: C=CC(=O)OCCCCCCOc1ccc(OC(=O)C2CCC(C(=O)Oc3ccc(OC(=O)C4CCC(C(=O)Oc5ccc(OCCCCCCOC(=O)C=C)cc5)CC4)c4sc(-c5cc6c(C)cc(C)cc6o5)nc34)CC2)cc1]
Specific compound (A-1) 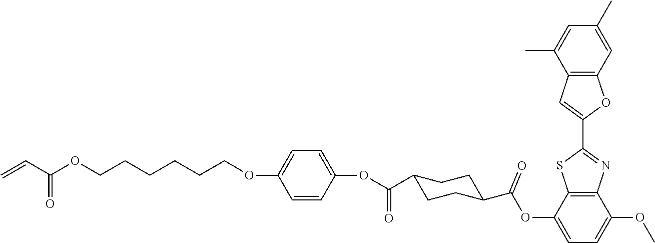 [SMILES: C=CC(=O)OCCCCCCOc1ccc(OC(=O)C2CCC(C(=O)Oc3ccc(OC)c4nc(-c5cc6c(C)cc(C)cc6o5)sc34)CC2)cc1]
Polymerizable compound (D-1) 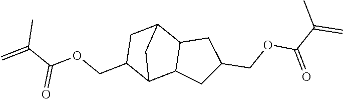 [SMILES: C=C(C)C(=O)OCC1CC2C3CC(COC(=O)C(=C)C)C(C3)C2C1]
Polymerization initiator S1 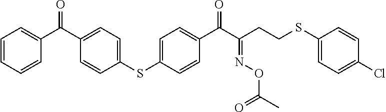 [SMILES: CC(=O)O/N=C(\CCSc1ccc(Cl)cc1)C(=O)c1ccc(Sc2ccc(C(=O)c3ccccc3)cc2)cc1]
Leveling agent P1 [a, b, and c in the formula each indicate the content (% by mass) of each repeating unit with respect to all repeating units, and a = 44.8, b = 50.3, and c = 4.9]

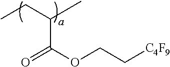

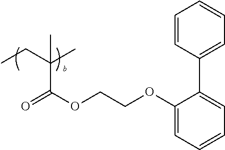

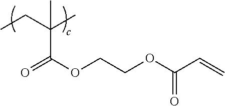

### Example 2

A liquid crystal composition 2 having the following formulation was prepared.

TABLE
Liquid crystal composition 2

| | |
|---|---|
| Liquid crystal compound (B-5) shown below | 222.5 parts by mass |
| Specific compound (A-23) shown below | 47.0 parts by mass |
| Polymerizable compound (D-1) shown above | 3.0 parts by mass |
| Polymerization initiator S1 shown above | 1.5 parts by mass |
| Leveling agent P1 shown above | 0.1 parts by mass |
| Cyclopentanone | 92.5 parts by mass |
| Methyl ethyl ketone | 64.2 parts by mass |

Liquid crystal compound (B-5) 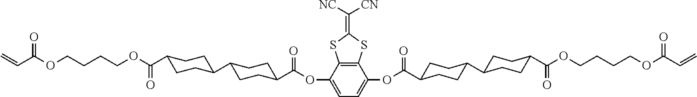 [SMILES: C=CC(=O)OCCCCOC(=O)C1CCC(C2CCC(C(=O)Oc3ccc(OC(=O)C4CCC(C5CCC(C(=O)OCCCCOC(=O)C=C)CC5)CC4)c4c3SC(=C(C#N)C#N)S4)CC2)CC1]
Specific compound (A-23)

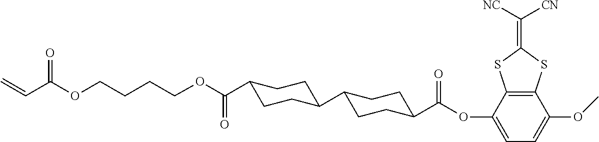 [SMILES: C=CC(=O)OCCCCOC(=O)C1CCC(C2CCC(C(=O)Oc3ccc(OC)c4c3SC(=C(C#N)C#N)S4)CC2)CC1]

### Example 3

A liquid crystal composition 3 having the following formulation was prepared.

TABLE
Liquid crystal composition 3

| | |
|---|---|
| Liquid crystal compound (B-1) shown above | 222.5 parts by mass |
| Specific compound (A-3) shown below | 47.0 parts by mass |
| Polymerizable compound (D-1) shown above | 3.0 parts by mass |
| Polymerization initiator S1 shown above | 1.5 parts by mass |
| Leveling agent P1 shown above | 0.1 parts by mass |
| Cyclopentanone | 92.5 parts by mass |
| Methyl ethyl ketone | 64.2 parts by mass |

Specific compound (A-3)

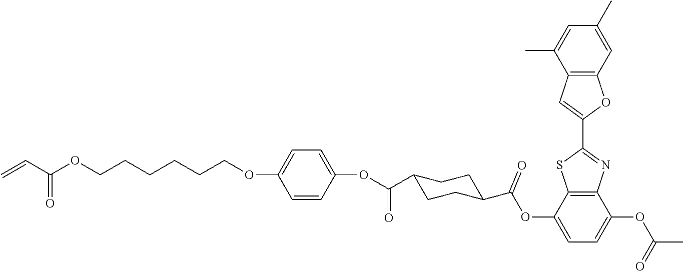 [SMILES: C=CC(=O)OCCCCCCOc1ccc(OC(=O)C2CCC(C(=O)Oc3ccc(OC(C)=O)c4nc(-c5cc6c(C)cc(C)cc6o5)sc34)CC2)cc1]

### Example 4

A liquid crystal composition 4 having the following formulation was prepared.

Liquid crystal composition 4

| | |
|---|---|
| Liquid crystal compound (B-1) shown above | 222.5 parts by mass |
| Specific compound (A-2) shown below | 47.0 parts by mass |
| Polymerizable compound (D-1) shown above | 3.0 parts by mass |
| Polymerization initiator S1 shown above | 1.5 parts by mass |
| Leveling agent P1 shown above | 0.1 parts by mass |
| Cyclopentanone | 92.5 parts by mass |
| Methyl ethyl ketone | 64.2 parts by mass |

Specific compound (A-2)

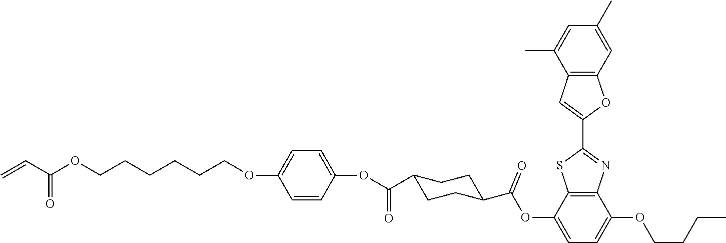 [SMILES: C=CC(=O)OCCCCCCOc1ccc(OC(=O)C2CCC(C(=O)Oc3ccc(OCCCC)c4nc(-c5cc6c(C)cc(C)cc6o5)sc34)CC2)cc1]

### Example 5

A liquid crystal composition 5 having the following formulation was prepared.

Liquid crystal composition 5

| | | |
|---|---|---|
| Liquid crystal compound (B-1) shown above | 222.5 | parts by mass |
| Specific compound (A-6) shown below | 47.0 | parts by mass |
| Polymerizable compound (D-1) shown above | 3.0 | parts by mass |
| Polymerization initiator S1 shown above | 1.5 | parts by mass |
| Leveling agent Pl shown above | 0.1 | parts by mass |
| Cyclopentanone | 92.5 | parts by mass |
| Methyl ethyl ketone | 64.2 | parts by mass |

Specific compound (A-6)

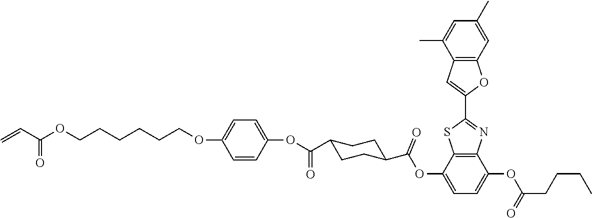 [SMILES: C=CC(=O)OCCCCCCOc1ccc(OC(=O)C2CCC(C(=O)Oc3ccc(OC(=O)CCCC)c4nc(-c5cc6c(C)cc(C)cc6o5)sc34)CC2)cc1]

### Example 6

A liquid crystal composition 6 having the following formulation was prepared.

Liquid crystal composition 6

| | |
|---|---|
| Liquid crystal compound (B-1) shown above | 222.5 parts by mass |
| Specific compound (A-1) shown above | 55.6 parts by mass |
| Polymerizable compound (D-1) shown above | 3.0 parts by mass |
| Polymerization initiator S1 shown above | 1.5 parts by mass |
| Leveling agent P1 shown above | 0.1 parts by mass |
| Cyclopentanone | 92.5 parts by mass |
| Methyl ethyl ketone | 64.2 parts by mass |

### Example 7

A liquid crystal composition 7 having the following formulation was prepared.

Liquid crystal composition 7

| | | |
|---|---|---|
| Liquid crystal compound (B-5) shown below | 222.5 | parts by mass |
| Specific compound (A-1) shown above | 47.0 | parts by mass |
| Polymerizable compound (D-1) shown above | 3.0 | parts by mass |
| Polymerization initiator S1 shown above | 1.5 | parts by mass |
| Leveling agent P1 shown above | 0.1 | parts by mass |
| Cyclopentanone | 92.5 | parts by mass |
| Methyl ethyl ketone | 64.2 | parts by mass |

Liquid crystal compound (B-5)

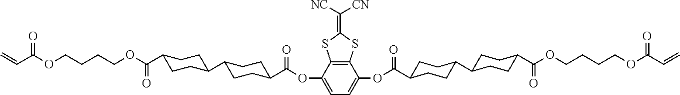 [SMILES: C=CC(=O)OCCCCOC(=O)C1CCC(C2CCC(C(=O)Oc3ccc(OC(=O)C4CCC(C5CCC(C(=O)OCCCCOC(=O)C=C)CC5)CC4)c4c3SC(=C(C#N)C#N)S4)CC2)CC1]

### Example 8

A liquid crystal composition 8 having the following formulation was prepared.

Liquid crystal composition 8

| | |
|---|---|
| Liquid crystal compound (B-1) shown above | 222.5 parts by mass |
| Specific compound (A-23) shown above | 47.0 parts by mass |
| Polymerizable compound (D-1) shown above | 3.0 parts by mass |
| Polymerization initiator S1 shown above | 1.5 parts by mass |
| Leveling agent P1 shown above | 0.1 parts by mass |
| Cyclopentanone | 92.5 parts by mass |
| Methyl ethyl ketone | 64.2 parts by mass |

### Example 9

A liquid crystal composition 9 having the following formulation was prepared.

Liquid crystal composition 9

| | | |
|---|---|---|
| Liquid crystal compound (B-2) shown below | 222.5 | parts by mass |
| Specific compound (A-7) shown below | 47.0 | parts by mass |
| Polymerizable compound (D-1) shown above | 3.0 | parts by mass |
| Polymerization initiator S1 shown above | 1.5 | parts by mass |
| Leveling agent P1 shown above | 0.1 | parts by mass |
| Cyclopentanone | 92.5 | parts by mass |
| Methyl ethyl ketone | 64.2 | parts by mass |

Liquid crystal compound (B-2) 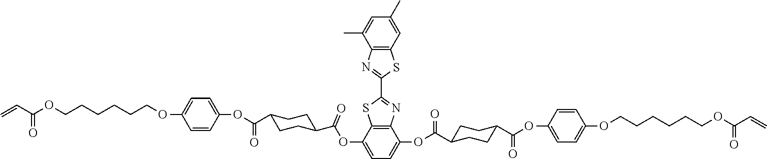 [SMILES: C=CC(=O)OCCCCCCOc1ccc(OC(=O)C2CCC(C(=O)Oc3ccc(OC(=O)C4CCC(C(=O)Oc5ccc(OCCCCCCOC(=O)C=C)cc5)CC4)c4sc(-c5nc6c(C)cc(C)cc6s5)nc34)CC2)cc1]
Specific compound (A-7)

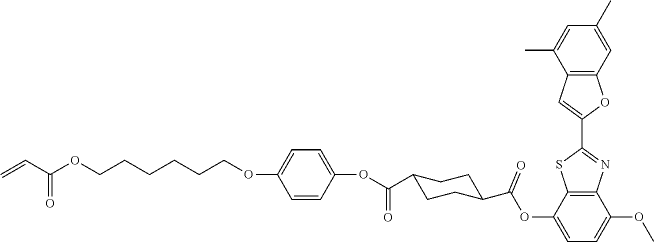 [SMILES: C=CC(=O)OCCCCCCOc1ccc(OC(=O)C2CCC(C(=O)Oc3ccc(OC)c4nc(-c5cc6c(C)cc(C)cc6o5)sc34)CC2)cc1]

### Example 10

A liquid crystal composition 10 having the following formulation was prepared.

Liquid crystal composition 10

| | | |
|---|---|---|
| Liquid crystal compound (B-2) shown above | 222.5 | parts by mass |
| Specific compound (A-8) shown below | 47.0 | parts by mass |
| Polymerizable compound (D-1) shown above | 3.0 | parts by mass |
| Polymerization initiator S1 shown above | 1.5 | parts by mass |
| Leveling agent P1 shown above | 0.1 | parts by mass |
| Cyclopentanone | 92.5 | parts by mass |
| Methyl ethyl ketone | 64.2 | parts by mass |

Specific compound (A-8)

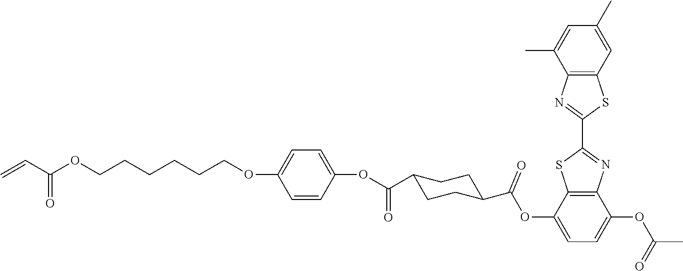 [SMILES: C=CC(=O)OCCCCCCOc1ccc(OC(=O)C2CCC(C(=O)Oc3ccc(OC(C)=O)c4nc(-c5nc6c(C)cc(C)cc6s5)sc34)CC2)cc1]

### Example 11

A liquid crystal composition 11 having the following formulation was prepared.

Liquid crystal composition 11

| | | |
|---|---|---|
| Liquid crystal compound (B-2) shown above | 222.5 | parts by mass |
| Specific compound (A-9) shown below | 47.0 | parts by mass |
| Polymerizable compound (D-1) shown above | 3.0 | parts by mass |
| Polymerization initiator S1 shown above | 1.5 | parts by mass |
| Leveling agent P1 shown above | 0.1 | parts by mass |
| Cyclopentanone | 92.5 | parts by mass |
| Methyl ethyl ketone | 64.2 | parts by mass |

Specific compound (A-9)

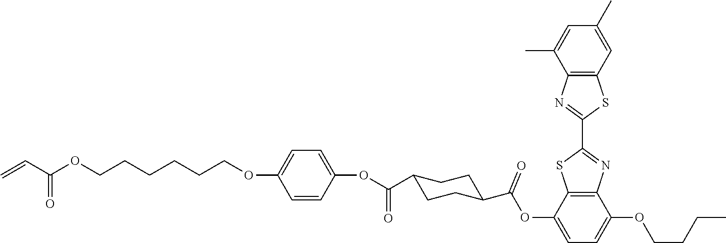 [SMILES: C=CC(=O)OCCCCCCOc1ccc(OC(=O)C2CCC(C(=O)Oc3ccc(OCCCC)c4nc(-c5nc6c(C)cc(C)cc6s5)sc34)CC2)cc1]

### Example 12

A liquid crystal composition 12 having the following formulation was prepared.

Liquid crystal composition 12

| | | |
|---|---|---|
| Liquid crystal compound (B-2) shown above | 222.5 | parts by mass |
| Specific compound (A-10) shown below | 47.0 | parts by mass |
| Polymerizable compound (D-1) shown above | 3.0 | parts by mass |
| Polymerization initiator S1 shown above | 1.5 | parts by mass |
| Leveling agent P1 shown above | 0.1 | parts by mass |
| Cyclopentanone | 92.5 | parts by mass |
| Methyl ethyl ketone | 64.2 | parts by mass |

Specific compound (A-10)

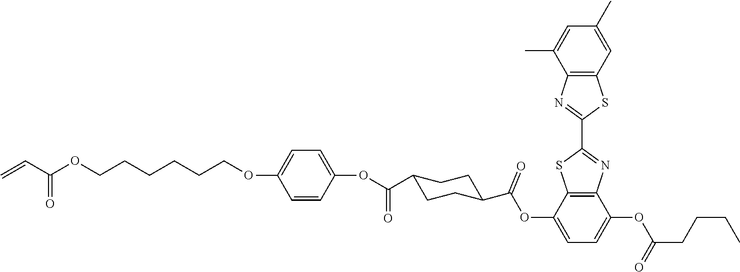 [SMILES: C=CC(=O)OCCCCCCOc1ccc(OC(=O)C2CCC(C(=O)Oc3ccc(OC(=O)CCCC)c4nc(-c5nc6c(C)cc(C)cc6s5)sc34)CC2)cc1]

### Example 13

A liquid crystal composition 13 having the following formulation was prepared.

Liquid crystal composition 13

| | | |
|---|---|---|
| Liquid crystal compound (B-3) shown below | 222.5 | parts by mass |
| Specific compound (A-11) shown below | 47.0 | parts by mass |
| Polymerizable compound (D-1) shown above | 3.0 | parts by mass |
| Polymerization initiator S1 shown above | 1.5 | parts by mass |
| Leveling agent P1 shown above | 0.1 | parts by mass |
| Cyclopentanone | 0.1 | parts by mass |
| Methyl ethyl ketone | 64.2 | parts by mass |

Liquid crystal compound (B-3) 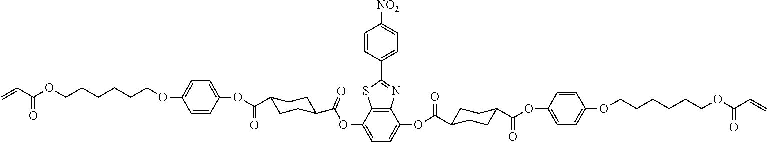 [SMILES: C=CC(=O)OCCCCCCOc1ccc(OC(=O)C2CCC(C(=O)Oc3ccc(OC(=O)C4CCC(C(=O)Oc5ccc(OCCCCCCOC(=O)C=C)cc5)CC4)c4sc(-c5ccc([N+](=O)[O-])cc5)nc34)CC2)cc1]
Specific compound (A-11)

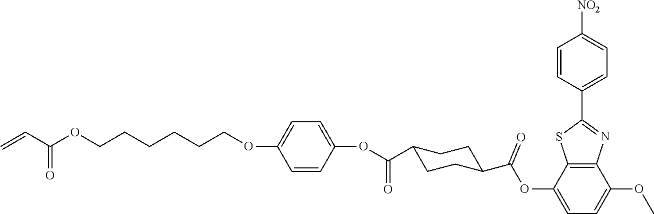 [SMILES: C=CC(=O)OCCCCCCOc1ccc(OC(=O)C2CCC(C(=O)Oc3ccc(OC)c4nc(-c5ccc([N+](=O)[O-])cc5)sc34)CC2)cc1]

### Example 14

A liquid crystal composition 14 having the following formulation was prepared.

Liquid crystal composition 14

| | | |
|---|---|---|
| Liquid crystal compound (B-3) shown above | 222.5 | parts by mass |
| Specific compound (A-12) shown below | 47.0 | parts by mass |
| Polymerizable compound (D-1) shown above | 3.0 | parts by mass |
| Polymerization initiator S1 shown above | 1.5 | parts by mass |
| Leveling agent P1 shown above | 0.1 | parts by mass |
| Cyclopentanone | 92.5 | parts by mass |
| Methyl ethyl ketone | 64.2 | parts by mass |

Specific compound (A-12)

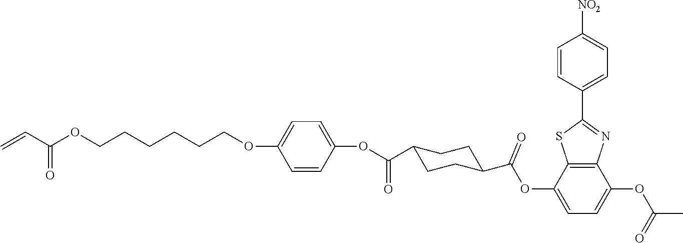 [SMILES: C=CC(=O)OCCCCCCOc1ccc(OC(=O)C2CCC(C(=O)Oc3ccc(OC(C)=O)c4nc(-c5ccc([N+](=O)[O-])cc5)sc34)CC2)cc1]

### Example 15

A liquid crystal composition 15 having the following formulation was prepared.

Liquid crystal composition 15

| | | |
|---|---|---|
| Liquid crystal compound (B-3) shown above | 222.5 | parts by mass |
| Specific compound (A-13) shown below | 47.0 | parts by mass |
| Polymerizable compound (D-1) shown above | 3.0 | parts by mass |
| Polymerization initiator S1 shown above | 1.5 | parts by mass |
| Leveling agent P1 shown above | 0.1 | parts by mass |
| Cyclopentanone | 925 | parts by mass |
| Methyl ethyl ketone | 64.2 | parts by mass |

Specific compound (A-13)

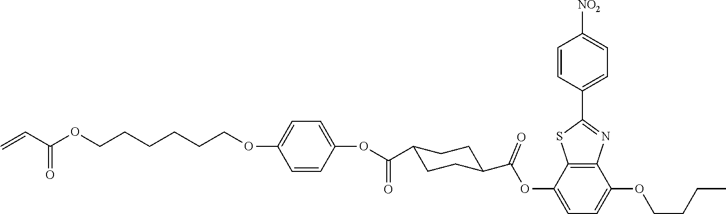 [SMILES: C=CC(=O)OCCCCCCOc1ccc(OC(=O)C2CCC(C(=O)Oc3ccc(OCCCC)c4nc(-c5ccc([N+](=O)[O-])cc5)sc34)CC2)cc1]

### Example 16

A liquid crystal composition 16 having the following formulation was prepared.

Liquid crystal composition 16

| | | |
|---|---|---|
| Liquid crystal compound (B-3) shown above | 222.5 | parts by mass |
| Specific compound (A-14) shown below | 47.0 | parts by mass |
| Polymerizable compound (D-1) shown above | 3.0 | parts by mass |
| Polymerization initiator S1 shown above | 1.5 | parts by mass |
| Leveling agent P1 shown above | 0.1 | parts by mass |
| Cyclopentanone | 92.5 | parts by mass |
| Methyl ethyl ketone | 64.2 | parts by mass |

Specific compound (A-14)

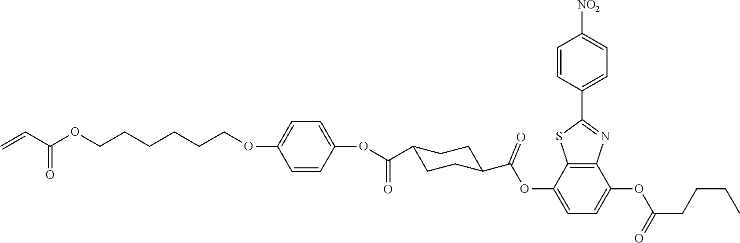 [SMILES: C=CC(=O)OCCCCCCOc1ccc(OC(=O)C2CCC(C(=O)Oc3ccc(OC(=O)CCCC)c4nc(-c5ccc([N+](=O)[O-])cc5)sc34)CC2)cc1]

### Example 17

A liquid crystal composition 17 having the following formulation was prepared.

Liquid crystal composition 17

| | | |
|---|---|---|
| Liquid crystal compound (B-4) shown below | 222.5 | parts by mass |
| Specific compound (A-16) shown below | 47.0 | parts by mass |
| Polymerizable compound (D-1) shown above | 3.0 | parts by mass |
| Polymerization initiator S1 shown above | 1.5 | parts by mass |
| Leveling agent Pl shown above | 0.1 | parts by mass |
| Cyclopentanone | 92.5 | parts by mass |
| Methyl ethyl ketone | 64.2 | parts by mass |

Liquid crystal compound (B-4) 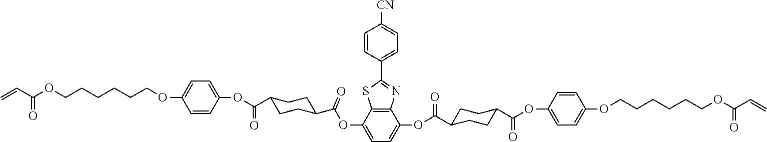 [SMILES: C=CC(=O)OCCCCCCOc1ccc(OC(=O)C2CCC(C(=O)Oc3ccc(OC(=O)C4CCC(C(=O)Oc5ccc(OCCCCCCOC(=O)C=C)cc5)CC4)c4sc(-c5ccc(C#N)cc5)nc34)CC2)cc1]
Specific compound (A-16)

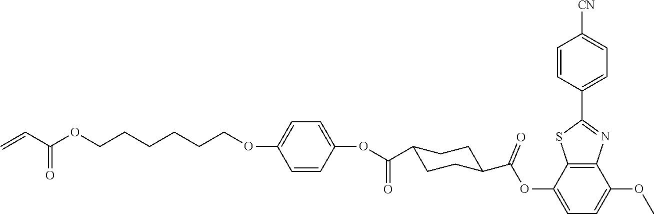 [SMILES: C=CC(=O)OCCCCCCOc1ccc(OC(=O)C2CCC(C(=O)Oc3ccc(OC)c4nc(-c5ccc(C#N)cc5)sc34)CC2)cc1]

### Example 18

A liquid crystal composition 18 having the following formulation was prepared.

Liquid crystal composition 18

| | | |
|---|---|---|
| Liquid crystal compound (B-4) shown above | 222.5 | parts by mass |
| Specific compound (A-17) shown below | 47.0 | parts by mass |
| Polymerizable compound (D-1) shown above | 3.0 | parts by mass |
| Polymerization initiator S1 shown above | 1.5 | parts by mass |
| Leveling agent P1 shown above | 0.1 | parts by mass |
| Cyclopentanone | 92.5 | parts by mass |
| Methyl ethyl ketone | 64.2 | parts by mass |

Specific compound (A-17)

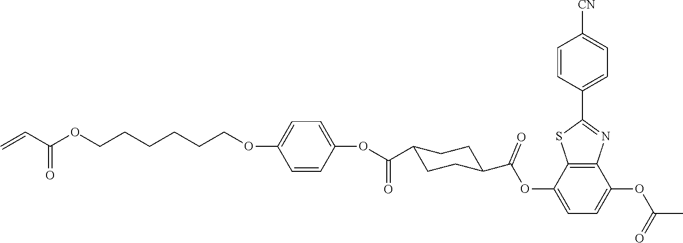 [SMILES: C=CC(=O)OCCCCCCOc1ccc(OC(=O)C2CCC(C(=O)Oc3ccc(OC(C)=O)c4nc(-c5ccc(C#N)cc5)sc34)CC2)cc1]

### Example 19

A liquid crystal composition 19 having the following formulation was prepared.

Liquid crystal composition 19

| | | |
|---|---|---|
| Liquid crystal compound (B-4) shown above | 222.5 | parts by mass |
| Specific compound (A-18) shown below | 47.0 | parts by mass |
| Polymerizable compound (D-1) shown above | 3.0 | parts by mass |
| Polymerization initiator S1 shown above | 1.5 | parts by mass |
| Leveling agent Pl shown above | 0.1 | parts by mass |
| Cyclopentanone | 92.5 | parts by mass |
| Methyl ethyl ketone | 64.2 | parts by mass |

Specific compound (A-18)

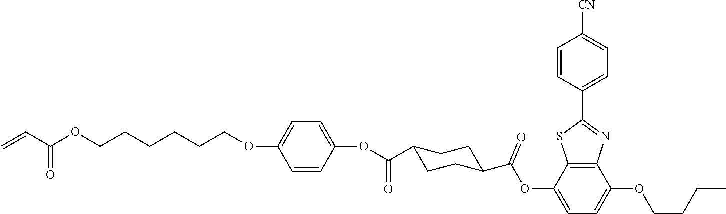 [SMILES: C=CC(=O)OCCCCCCOc1ccc(OC(=O)C2CCC(C(=O)Oc3ccc(OCCCC)c4nc(-c5ccc(C#N)cc5)sc34)CC2)cc1]

### Example 20

A liquid crystal composition 20 having the following formulation was prepared.

Liquid crystal composition 20

| | | |
|---|---|---|
| Liquid crystal compound (B-4) shown above | 222.5 | parts by mass |
| Specific compound (A-19) shown below | 47.0 | parts by mass |
| Polymerizable compound (D-1) shown above | 3.0 | parts by mass |
| Polymerization initiator S1 shown above | 1.5 | parts by mass |
| Leveling agent P1 shown above | 0.1 | parts by mass |
| Cyclopentanone | 92.5 | parts by mass |
| Methyl ethyl ketone | 64.2 | parts by mass |

Specific compound (A-19)

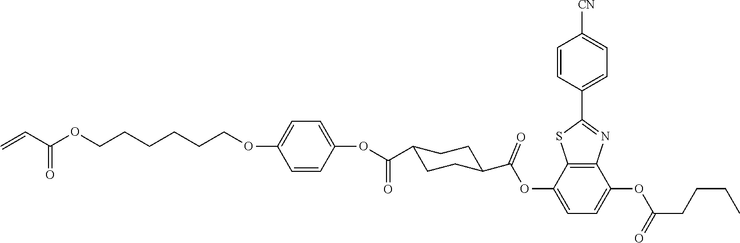 [SMILES: C=CC(=O)OCCCCCCOc1ccc(OC(=O)C2CCC(C(=O)Oc3ccc(OC(=O)CCCC)c4nc(-c5ccc(C#N)cc5)sc34)CC2)cc1]

### Comparative Example 1

A liquid crystal composition C-1 having the following formulation was prepared.

Liquid crystal composition C-1

| | |
|---|---|
| Liquid crystal compound (B-1) shown above | 269.5 parts by mass |
| Polymerizable compound (D-1) shown above | 3.0 parts by mass |
| Polymerization initiator S1 shown above | 1.5 parts by mass |
| Leveling agent P1 shown above | 0.1 parts by mass |
| Cyclopentanone | 92.5 parts by mass |
| Methyl ethyl ketone | 64.2 parts by mass |

### Comparative Example 2

A liquid crystal composition C-2 having the following formulation was prepared.

Liquid crystal composition C-2

| | | |
|---|---|---|
| Liquid crystal compound (B-1) shown above | 22.5 | parts by mass |
| Compound (CA-1) shown below | 47.0 | parts by mass |
| Polymerizable compound (D-1) shown above | 3.0 | parts by mass |
| Polymerization initiator S1 shown above | 1.5 | parts by mass |
| Leveling agent P1 shown above | 0.1 | parts by mass |
| Cyclopentanone | 92.5 | parts by mass |
| Methyl ethyl ketone | 64.2 | parts by mass |

Compound (CA-1)

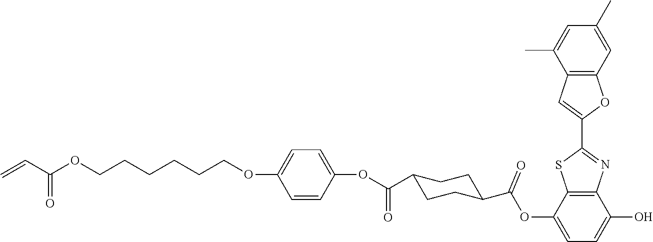 [SMILES: C=CC(=O)OCCCCCCOc1ccc(OC(=O)C2CCC(C(=O)Oc3ccc(O)c4nc(-c5cc6c(C)cc(C)cc6o5)sc34)CC2)cc1]

### [Production of Optical Film]

A glass substrate provided with a rubbing-treated polyimide alignment film (SE-150 manufactured by Nissan Chemical Corporation) was coated with the liquid crystal composition 1 prepared in Example 1 by spin coating. The coating film was subjected to an alignment treatment at 200° C. to form a liquid crystal layer. Thereafter, the liquid crystal layer was cooled to 135° C. and subjected to alignment fixation by irradiation with ultraviolet rays at 1,000 mJ/cm² to form a liquid crystal cured layer, thereby producing an optical film 1 for measuring a wavelength dispersion.

The phase difference of the optical film 1 was measured, and it was confirmed that Re(450)/Re(550)=0.88.

By the same method, the liquid crystal cured layer was formed and the optical film was produced from the liquid crystal compositions prepared in Examples 2 to 20 and Comparative Examples 1 and 2.

### [Evaluation of Precipitation]

20 μL of the liquid crystal compositions 1 to 20, C-1, and C-2 prepared as described above was dropped onto a glass plate and allowed to stand at 25° C. for 1 hour, and the precipitation of a solid film was visually observed. Specifically, the area of the white turbid portion (whitened portion) was estimated with respect to the area of the solid film, and precipitation was determined by an indicator of 1 to 3 according to the following determination criterion. The results are shown in Table 4.

### <Precipitation Indicator>

Precipitation 1: whitening in a portion of 75% or more and 100% or less Precipitation 2: whitening in a portion of 25% or more and less than 75% Precipitation 3: whitening in a portion of less than 25%

### [Evaluation of Aligning Properties]

Aligning properties were measured by setting a light emitting diode (LED) light source, a lower polarizing plate, a liquid crystal cured layer (optical film produced from each liquid crystal composition), and an upper polarizing plate in order from the bottom on a table such that each surface was horizontal. In this case, the sample and the upper polarizing plate were rotatable. Light emitted from the light source and transmitted through the lower polarizing plate, the sample, and the upper polarizing plate in this order was measured for brightness from a vertical direction using a brightness meter (BM-5A (manufactured by TOPCON Corporation)).

In the measurement, first, the upper polarizing plate was rotated in a state in which the sample was not present, and was adjusted to a position where the brightness was the darkest (crossed nicols state). The sample peeled off from the protective film was inserted between the polarizing plates, and the brightness at a minimum was measured by rotating the sample under the crossed nicols state. Next, the brightness at a maximum was measured by rotating the sample in a parallel nicols arrangement of two polarizing plates consisting of the upper polarizing plate and the lower polarizing plate.

In order to remove contribution of brightness leakage caused by the upper polarizing plate and the lower polarizing plate, a value obtained by the following expression was evaluated by the following evaluation standard. The results are shown in Table 4.

Aligning properties=1/((Minimum brightness under crossed nicols state at time of sample placement)/(Maximum brightness under parallel nicols state at time of sample placement)−(Minimum brightness under crossed nicols state in state in which sample was not present)/(Maximum brightness under parallel nicols state in state in which sample was not present))

### Evaluation Standard

A: the above-described aligning properties were 200,000 or more.
B: the above-described aligning properties were 100,000 or more and less than 200,000.
C: the above-described aligning properties were less than 100,000.

TABLE 4

| | Liquid crystal | Specific | Liquid crystal | Ratio※ | | Aligning |
|---|---|---|---|---|---|---|
| | composition | compound A | compound | (% by mass) | Precipitation | properties |
| Example 1 | 1 | A-1 | B-1 | 17.4 | 3 | A |
| Example 2 | 2 | A-23 | B-5 | 17.4 | 3 | A |
| Example 3 | 3 | A-3 | B-1 | 17.4 | 3 | A |
| Example 4 | 4 | A-2 | B-1 | 17.4 | 3 | A |
| Example 5 | 5 | A-6 | B-1 | 17.4 | 3 | A |
| Example 6 | 6 | A-1 | B-1 | 20 | 3 | B |
| Example 7 | 7 | A-1 | B-5 | 17.4 | 3 | B |
| Example 8 | 8 | A-23 | B-1 | 17.4 | 3 | B |
| Example 9 | 9 | A-7 | B-2 | 17.4 | 3 | A |
| Example 10 | 10 | A-8 | B-2 | 17.4 | 3 | A |
| Example 11 | 11 | A-9 | B-2 | 17.4 | 3 | A |
| Example 12 | 12 | A-10 | B-2 | 17.4 | 3 | A |
| Example 13 | 13 | A-11 | B-3 | 17.4 | 3 | A |
| Example 14 | 14 | A-12 | B-3 | 17.4 | 3 | A |
| Example 15 | 15 | A-13 | B-3 | 17.4 | 3 | A |
| Example 16 | 16 | A-14 | B-3 | 17.4 | 3 | A |
| Example 17 | 17 | A-16 | B-4 | 17.4 | 3 | A |
| Example 18 | 18 | A-17 | B-4 | 17.4 | 3 | A |
| Example 19 | 19 | A-18 | B-4 | 17.4 | 3 | A |
| Example 20 | 20 | A-19 | B-4 | 17.4 | 3 | A |
| Comparative | C-1 | — | B-1 | — | 1 | A |
| Example 1 | | | | | | |
| Comparative | C-2 | CA-1 | B-1 | 17.4 | 3 | C |
| Example 2 | | | | | | |

※Mass ratio of specific compound with respect to total mass of specific compound A and liquid crystal compound

From the results shown in Table 4, it was found that, in a case where the specific compound A was not blended, the precipitation could not be suppressed (Comparative Example 1).

In addition, it was found that, in a case where a compound not corresponding to the compound represented by Formula (A) described above was blended, the precipitation was suppressed, but the aligning properties were deteriorated (Comparative Example 2).

On the other hand, it was found that, in the liquid crystal composition containing the specific compound A and the liquid crystal compound, the precipitation was suppressed and the aligning properties were also favorable (Examples 1 to 20).

In particular, from the comparison between Example 1 and Example 6, it was found that, in a case where the content of the specific compound A was 18% by mass or less with respect to the total mass of the specific compound A and the liquid crystal compound, the aligning properties were more favorable.

In addition, from the comparison between Example 1 and Example 7, and the comparison between Example 2 and Example 8, it was found that, in a case where the compound represented by Formula (A) (specific compound A) and the compound represented by Formula (B) (liquid crystal compound) had the same structure in which Ar¹ in Formula (A) and Ar² in Formula (B) had the same structure, G¹¹ in Formula (A) and G¹ in Formula (B) had the same structure, and A¹¹ in Formula (A) and A¹ in Formula (B) had the same structure, the aligning properties were more favorable.

### EXPLANATION OF REFERENCES

10: optical film
12: liquid crystal cured layer
14: alignment film 16: support

What is claimed is:

1. A liquid crystal composition comprising:
a compound represented by Formula (A); and
a liquid crystal compound, 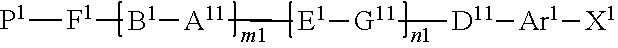
in Formula (A),
P¹ represents a polymerizable group,
F¹ represents a spacer group,
B¹, E¹, and D¹¹ each independently represent a single bond or a divalent linking group,
A¹¹ and G¹¹ each independently represent a divalent aromatic hydrocarbon group having 6 to 20 carbon atoms or a divalent alicyclic hydrocarbon group having 3 to 16 carbon atoms, provided that, hydrogen atoms in the aromatic hydrocarbon group or the alicyclic hydrocarbon group may be each independently replaced with a halogen atom, —R^{L1}, —OR^{L1}, a cyano group, or a nitro group, where R^{L1} represents an alkyl group having 1 to 4 carbon atoms and hydrogen atoms in the alkyl group may be each independently replaced with a fluorine atom, and carbon atoms in the aromatic hydrocarbon group or the alicyclic hydrocarbon group may be each independently replaced with an oxygen atom, a sulfur atom, or a nitrogen atom,
m1 and n1 each independently represent an integer of 0 to 3, provided that, in a case where m1 is 2 or 3, a plurality of B1's may be the same or different from each other and a plurality of A¹¹'s may be the same or different from each other, and in a case where n1 is 2 or 3, a plurality of E¹'s may be the same or different from each other and a plurality of G¹¹'s may be the same or different from each other,
X¹ represents —OR^{L2}, —O—(C—O)—R^{L3}, —O—(C═O)—OR^{L4}, —O—(C═O)—NH—R^{L5}, —O—(C═O)—NR^{L6}—R^{L7}, or —O—(C═S)—R^{L8}, where R^{L2} represents an alkyl group having 1 to 4 carbon atoms, an acryloyl group, or a methacryloyl group, R^{L3} represents an alkyl group having 1 to 4 carbon atoms, and R^{L4} to R^{L8} each independently represent a non-cyclic alkyl group, a monovalent aromatic group, or a monovalent alicyclic group, and
Ar¹ represents any aromatic ring selected from the group consisting of groups represented by Formulae (Ar-1) to (Ar-3), 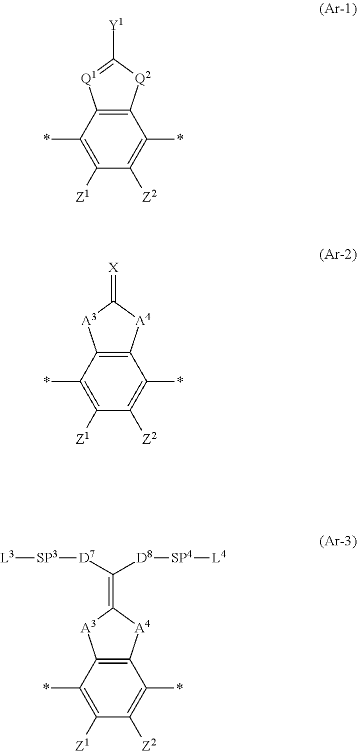
in Formulae (Ar-1) to (Ar-3),
* represents a bonding position to D¹¹ or X¹,
Q¹ represents N or CH,
Q² represents —S—, —O—, or —N(R⁶)—, where R⁶ represents a hydrogen atom or an alkyl group having 1 to 6 carbon atoms,
Y¹ represents an aromatic hydrocarbon group having 6 to 12 carbon atoms, which may have a substituent, an aromatic heterocyclic group having 3 to 12 carbon atoms, which may have a substituent, or an alicyclic hydrocarbon group having 6 to 20 carbon atoms, which may have a substituent, where one or more of —CH₂—'s constituting the alicyclic hydrocarbon group may be replaced with —O—, —S—, or —NH—,
Z¹ and Z² each independently represent a hydrogen atom, a monovalent aliphatic hydrocarbon group having 1 to 20 carbon atoms, a monovalent alicyclic hydrocarbon group having 3 to 20 carbon atoms, a monovalent aromatic hydrocarbon group having 6 to 20 carbon atoms, a monovalent aromatic heterocyclic group having 6 to 20 carbon atoms, a halogen atom, a cyano group, a nitro group, —OR⁷, —NR⁸R⁹, —SR¹⁰, —COOR¹¹, or —COR¹², where R⁷ to R¹² each independently represent a hydrogen atom or an alkyl group having 1 to 6 carbon atoms and Z¹ and Z² may be bonded to each other to form an aromatic ring,
A³ and A⁴ each independently represent a group selected from the group consisting of —O—, —N(R¹³)—, —S—, and —CO—, where R¹³ represents a hydrogen atom or a substituent,
X represents a non-metal atom of Groups 14 to 16, provided that a hydrogen atom or a substituent may be bonded to the non-metal atom,
D⁷ and D⁸ each independently represent a single bond, —CO—, —O—, —S—, —C(═S)—, —CR¹R²—, —CR³—CR⁴—, —NR⁵—, or a divalent linking group consisting of a combination of two or more of these groups, where R¹ to R⁵ each independently represent a hydrogen atom, a fluorine atom, or an alkyl group having 1 to 12 carbon atoms,
SP³ and SP⁴ each independently represent a single bond or a divalent aliphatic hydrocarbon group having 1 to 20 carbon atoms, provided that one or more of —CH₂—'s constituting the aliphatic hydrocarbon group may be replaced with —O—, —S—, —NH—, —N(Q)-, or —CO—, where Q represents a substituent, and
L³ and L⁴ each independently represent a monovalent organic group.

2. The liquid crystal composition according to claim 1,
wherein the liquid crystal compound is a compound represented by Formula (B), 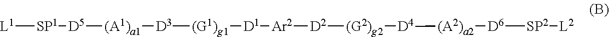
in Formula (B),
a1, a2, g1, and g2 each independently represent 0 or 1, provided that at least one of a1 or g1 represents 1 and at least one of a2 or g2 represents 1,
D¹, D², D³, D⁴, D⁵, and D⁶ each independently represent a single bond, —CO—, —O—, —S—, —C(═S)—, —CR¹R²—, —CR³═CR⁴—, —NR⁵—, or a divalent linking group consisting of a combination of two or more of these groups, where R¹ to R⁵ each independently represent a hydrogen atom, a fluorine atom, or an alkyl group having 1 to 12 carbon atoms,
G¹ and G² each independently represent an aromatic ring having 6 to 20 carbon atoms, which may have a substituent, or a divalent alicyclic hydrocarbon group having 5 to 20 carbon atoms, which may have a substituent, where one or more of —CH₂—'s constituting the alicyclic hydrocarbon group may be replaced with —O—, —S—, or —NH—,
A¹ and A² each independently represent an aromatic ring having 6 to 20 carbon atoms, which may have a substituent, or a divalent alicyclic hydrocarbon group having 5 to 20 carbon atoms, which may have a substituent, where one or more of —CH₂—'s constituting the alicyclic hydrocarbon group may be replaced with —O—, —S—, or —NH—,
SP¹ and SP² each independently represent a single bond or a divalent aliphatic hydrocarbon group having 1 to 20 carbon atoms, provided that one or more of —CH₂—'s constituting the aliphatic hydrocarbon group may be replaced with —O—, —S—, —NH—, —N(Q)-, or —CO—, where Q represents a substituent,
L¹ and L² each independently represent a monovalent organic group, where at least one of L¹ or L² represents a polymerizable group, provided that, in a case where Ar² is an aromatic ring represented by Formula (Ar-3), at least one of L¹, L², or L³ or L⁴ in Formula (Ar-3) represents a polymerizable group, and
Ar² represents any aromatic ring selected from the group consisting of groups represented by Formulae (Ar-1) to (Ar-7), 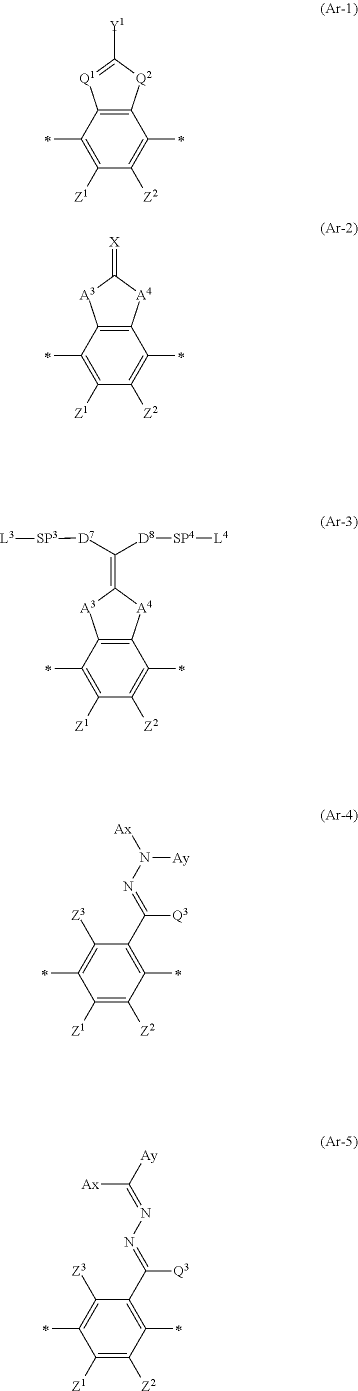 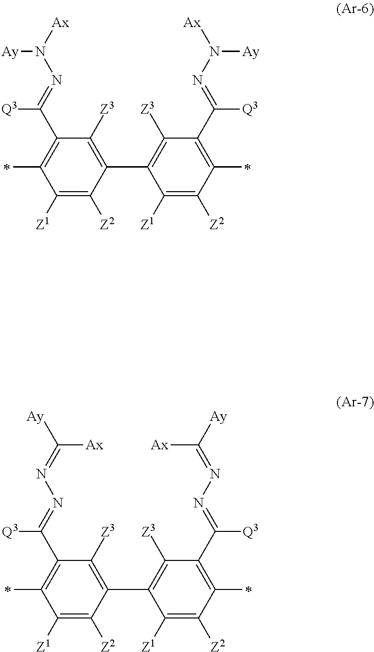
in Formulae (Ar-1) to (Ar-7),
* represents a bonding position to D¹ or D²,
Q¹ represents N or CH,
Q² represents —S—, —O—, or —N(R⁶)—, where R⁶ represents a hydrogen atom or an alkyl group having 1 to 6 carbon atoms,
Y¹ represents an aromatic hydrocarbon group having 6 to 12 carbon atoms, which may have a substituent, an aromatic heterocyclic group having 3 to 12 carbon atoms, which may have a substituent, or an alicyclic hydrocarbon group having 6 to 20 carbon atoms, which may have a substituent, where one or more of —CH₂—'s constituting the alicyclic hydrocarbon group may be replaced with —O—, —S—, or —NH—,
Z¹, Z², and Z³ each independently represent a hydrogen atom, a monovalent aliphatic hydrocarbon group having 1 to 20 carbon atoms, a monovalent alicyclic hydrocarbon group having 3 to 20 carbon atoms, a monovalent aromatic hydrocarbon group having 6 to 20 carbon atoms, a monovalent aromatic heterocyclic group having 6 to 20 carbon atoms, a halogen atom, a cyano group, a nitro group, —OR⁷, —NR⁸R⁹, —SR¹⁰, —COOR¹¹, or —COR¹², where R⁷ to R¹² each independently represent a hydrogen atom or an alkyl group having 1 to 6 carbon atoms and Z¹ and Z² may be bonded to each other to form an aromatic ring,
A³ and A⁴ each independently represent a group selected from the group consisting of —O—, —N(R¹³)—, —S—, and —CO—, where R¹³ represents a hydrogen atom or a substituent,
X represents a non-metal atom of Groups 14 to 16, provided that a hydrogen atom or a substituent may be bonded to the non-metal atom,
D⁷ and D⁸ each independently represent a single bond, —CO—, —O—, —S—, —C(═S)—, —CR¹R²—, —CR³—CR⁴—, —NR⁵—, or a divalent linking group consisting of a combination of two or more of these groups, where R¹ to R⁵ each independently represent a hydrogen atom, a fluorine atom, or an alkyl group having 1 to 12 carbon atoms,
SP³ and SP⁴ each independently represent a single bond or a divalent aliphatic hydrocarbon group having 1 to 20 carbon atoms, provided that one or more of —CH₂—'s constituting the aliphatic hydrocarbon group may be replaced with —O—, —S—, —NH—, —N(Q)-, or —CO—, where Q represents a substituent,
L³ and L⁴ each independently represent a monovalent organic group, where at least one of L³, L⁴, or L¹ or L² in Formula (B) represents a polymerizable group,
Ax represents an organic group having 2 to 30 carbon atoms, which has at least one aromatic ring selected from the group consisting of an aromatic hydrocarbon ring and an aromatic heterocyclic ring,
Ay represents a hydrogen atom, an alkyl group having 1 to 12 carbon atoms, which may have a substituent, or an organic group having 2 to 30 carbon atoms, which has at least one aromatic ring selected from the group consisting of an aromatic hydrocarbon ring and an aromatic heterocyclic ring,
where the aromatic rings in Ax and Ay may have a substituent, and Ax and Ay may be bonded to each other to form a ring, and
Q³ represents a hydrogen atom or an alkyl group having 1 to 20 carbon atoms, which may have a substituent.

3. The liquid crystal composition according to claim 2,
wherein a content of the compound represented by Formula (A) is 18% by mass or less with respect to a total mass of the compound represented by Formula (A) and the liquid crystal compound.

4. The liquid crystal composition according to claim 1,
wherein Ar¹ in Formula (A) is the aromatic ring represented by Formula (Ar-1) or (Ar-2).

5. The liquid crystal composition according to claim 2,
wherein Ar¹ in Formula (A) has the same structure as Ar² in Formula (B).

6. The liquid crystal composition according to claim 2,
wherein G¹¹ in Formula (A) has the same structure as G¹ in Formula (B), and
A¹¹ in Formula (A) has the same structure as A¹ in Formula (B).

7. A monofunctional monomer represented by Formula (A), 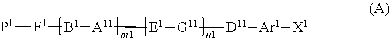
in Formula (A),
P¹ represents a polymerizable group,
F¹ represents a spacer group,
B¹, E¹, and D¹¹ each independently represent a single bond or a divalent linking group,
A¹¹ and G¹¹ each independently represent a divalent aromatic hydrocarbon group having 6 to 20 carbon atoms or a divalent alicyclic hydrocarbon group having 3 to 16 carbon atoms, provided that, hydrogen atoms in the aromatic hydrocarbon group or the alicyclic hydrocarbon group may be each independently replaced with a halogen atom, —R^{L1}, —OR^{L1}, a cyano group, or a nitro group, where R^{L1} represents an alkyl group having 1 to 4 carbon atoms and hydrogen atoms in the alkyl group may be each independently replaced with a fluorine atom, and carbon atoms in the aromatic hydrocarbon group or the alicyclic hydrocarbon group may be each independently replaced with an oxygen atom, a sulfur atom, or a nitrogen atom,
m1 and n1 each independently represent an integer of 0 to 3, provided that, in a case where m1 is 2 or 3, a plurality of B1's may be the same or different from each other and a plurality of A¹¹'s may be the same or different from each other, and in a case where n1 is 2 or 3, a plurality of E¹'s may be the same or different from each other and a plurality of G¹¹'s may be the same or different from each other,
X¹ represents —OR^{L2}, —O—(C—O)—R^{L3}, —O—(C═O)—OR^{L4}, —O—(C—O)—NH—R^{L5}, —O—(C—O)—NR^{L6}—R^{L7}, or —O—(C═S)—R^{L8}, where R^{L2} represents an alkyl group having 1 to 4 carbon atoms, an acryloyl group, or a methacryloyl group, R^{L3} represents an alkyl group having 1 to 4 carbon atoms, and R^{L4} to R^{L8} each independently represent a non-cyclic alkyl group, a monovalent aromatic group, or a monovalent alicyclic group, and
Ar¹ represents any aromatic ring selected from the group consisting of groups represented by Formulae (Ar-1) to (Ar-3), 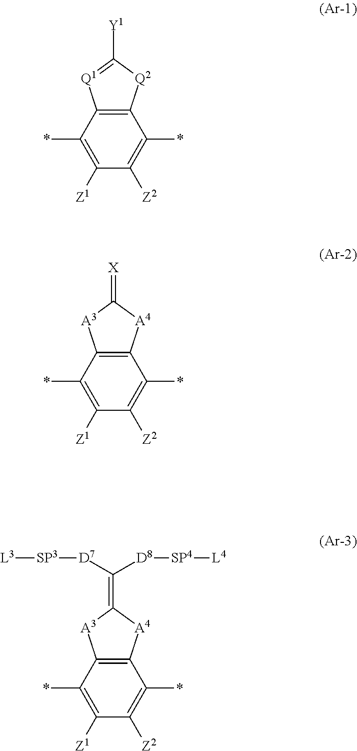
in Formulae (Ar-1) to (Ar-7),
* represents a bonding position to D¹¹ or X¹,
Q¹ represents N or CH,
Q² represents —S—, —O—, or —N(R⁶)—, where R⁶ represents a hydrogen atom or an alkyl group having 1 to 6 carbon atoms,
Y¹ represents an aromatic hydrocarbon group having 6 to 12 carbon atoms, which may have a substituent, an aromatic heterocyclic group having 3 to 12 carbon atoms, which may have a substituent, or an alicyclic hydrocarbon group having 6 to 20 carbon atoms, which may have a substituent, where one or more of —CH₂—'s constituting the alicyclic hydrocarbon group may be replaced with —O—, —S—, or —NH—,
Z¹ and Z² each independently represent a hydrogen atom, a monovalent aliphatic hydrocarbon group having 1 to 20 carbon atoms, a monovalent alicyclic hydrocarbon group having 3 to 20 carbon atoms, a monovalent aromatic hydrocarbon group having 6 to 20 carbon atoms, a monovalent aromatic heterocyclic group having 6 to 20 carbon atoms, a halogen atom, a cyano group, a nitro group, —OR⁷, —NR⁸R⁹, —SR¹⁰, —COOR¹¹, or —COR¹², where R⁷ to R¹² each independently represent a hydrogen atom or an alkyl group having 1 to 6 carbon atoms and Z¹ and Z² may be bonded to each other to form an aromatic ring,
A³ and A⁴ each independently represent a group selected from the group consisting of —O—, —N(R^{L3})—, —S—, and —CO—, where R¹³ represents a hydrogen atom or a substituent,
X represents a non-metal atom of Groups 14 to 16, provided that a hydrogen atom or a substituent may be bonded to the non-metal atom,
D⁷ and D⁸ each independently represent a single bond, —CO—, —O—, —S—, —C(═S)—, —CR¹R²—, —CR³═CR⁴—, —NR⁵—, or a divalent linking group consisting of a combination of two or more of these groups, where R¹ to R⁵ each independently represent a hydrogen atom, a fluorine atom, or an alkyl group having 1 to 12 carbon atoms,
SP³ and SP⁴ each independently represent a single bond or a divalent aliphatic hydrocarbon group having 1 to 20 carbon atoms, provided that one or more of —CH₂—'s constituting the aliphatic hydrocarbon group may be replaced with —O—, —S—, —NH—, —N(Q)-, or —CO—, where Q represents a substituent, and
L³ and L⁴ each independently represent a monovalent organic group.

8. A liquid crystal cured layer obtained by fixing an alignment state of the liquid crystal composition according to claim 1.

9. An optical film comprising:
the liquid crystal cured layer according to claim 8.

10. A polarizing plate comprising:
the optical film according to claim 9; and
a polarizer.

11. An image display device comprising:
the optical film according to claim 9.

12. The liquid crystal composition according to claim 1,
wherein a content of the compound represented by Formula (A) is 18% by mass or less with respect to a total mass of the compound represented by Formula (A) and the liquid crystal compound.

13. The liquid crystal composition according to claim 2,
wherein Ar¹ in Formula (A) is the aromatic ring represented by Formula (Ar-1) or (Ar-2).

14. The liquid crystal composition according to claim 3,
wherein Ar¹ in Formula (A) has the same structure as Ar² in Formula (B).

15. The liquid crystal composition according to claim 3,
wherein G¹¹ in Formula (A) has the same structure as G¹ in Formula (B), and
A¹¹ in Formula (A) has the same structure as A¹ in Formula (B).

16. A liquid crystal cured layer obtained by fixing an alignment state of the liquid crystal composition according to claim 2.

17. An optical film comprising:
the liquid crystal cured layer according to claim 16.

18. A polarizing plate comprising:
the optical film according to claim 17; and
a polarizer.

19. An image display device comprising:
the optical film according to claim 17.

20. The liquid crystal composition according to claim 3,
wherein Ar¹ in Formula (A) is the aromatic ring represented by Formula (Ar-1) or (Ar-2).
